SEQUENCE LISTING (30 sequences)

SEQ ID NO: 1 - DNA, 143 bp, Artificial Sequence
  Oligonucleotide
tctccacatttcgagcatcgtttatcgtaccctaaatctacggtagtaaatgtatgcttgtaataaatatgacgtcaaccgacatgtttttgtagactagtgcatgcaccggagatctgtaactctccataagcctagccatg

SEQ ID NO: 2 - DNA, 100 bp, Artificial Sequence
  Oligonucleotide
aagcgaggtacgatgaactggtaatgaaaaataaaaaaaggtaataattaattgctttagcattcaataattaacaacaaagtcaaaattcctccaacag

SEQ ID NO: 3 - DNA, 80 bp, Artificial Sequence
  Oligonucleotide
tgtttttgtagactagtgcatgcaccggagatctgtaactctccataagcctagccatgatgcacgtgaataattttccc

SEQ ID NO: 4 - DNA, 80 bp, Artificial Sequence
  Oligonucleotide
taataattaattgctttagcattcaataattaacaacaaagtcaaaattcctccaacagttacgtgtcaggagaacgatc

SEQ ID NO: 5 - DNA, 80 bp, Artificial Sequence
  Oligonucleotide
catgtttttgtagactagtgcatgcaccggagatctgtaactctccataagcctagccatggagtctccaaccaaggaaa

SEQ ID NO: 6 - DNA, 100 bp, Artificial Sequence
  Oligonucleotide
aatgaaaaataaaaaaaggtaataattaattgctttagcattcaataattaacaacaaagtcaaaattcctccaacagttatccgtagtctcctggcaag

SEQ ID NO: 7 - DNA, 80 bp, Artificial Sequence
  Oligonucleotide
acatgtttttgtagactagtgcatgcaccggagatctgtaactctccataagcctagccatggggcaggcatgcggccac

SEQ ID NO: 8 - DNA, 80 bp, Artificial Sequence
  Oligonucleotide
ataattaattgctttagcattcaataattaacaacaaagtcaaaattcctccaacagtcagccctcgaggctgcagcagc

SEQ ID NO: 9 - DNA, 80 bp, Artificial Sequence
  Oligonucleotide
acatgtttttgtagactagtgcatgcaccggagatctgtaactctccataagcctagccatgaggaaagacgagcgcgag

SEQ ID NO: 10 - DNA, 80 bp, Artificial Sequence
  Oligonucleotide
taataattaattgctttagcattcaataattaacaacaaagtcaaaattcctccaacagaggcctgaattcctcgaggtc

SEQ ID NO: 11 - DNA, 80 bp, Artificial Sequence
  Oligonucleotide
acatgtttttgtagactagtgcatgcaccggagatctgtaactctccataagcctagccatgcctctggttgacttcttc

SEQ ID NO: 12 - DNA, 80 bp, Artificial Sequence
  Oligonucleotide
aaattaaaaaaaaaaaataaaaatataatgaatatatgaccatgaccctgggatgctattaggcagggtctccacctgac

SEQ ID NO: 13 - DNA, 80 bp, Artificial Sequence
  Oligonucleotide
atgtttttgtagactagtgcatgcaccggagatctgtaactctccataagcctagccatgagccatggagcctccggccg

SEQ ID NO: 14 - DNA, 80 bp, Artificial Sequence
  Oligonucleotide
aataattaattgctttagcattcaataattaacaacaaagtcaaaattcctccaacagtcaggcagggtctccgcctgac

SEQ ID NO: 15 - DNA, 80 bp, Artificial Sequence
  Oligonucleotide
gacatgtttttgtagactagtgcatgcaccggagatctgtaactctccataagcctagccatgacagcaaaaaattctcc

SEQ ID NO: 16 - DNA, 80 bp, Artificial Sequence
  Oligonucleotide
attaaaaaaaaaaaataaaaatataatgaatatatgaccatgaccctgggatgctactaaactctagatattcaacaggc

SEQ ID NO: 17 - DNA, 79 bp, Artificial Sequence
  Oligonucleotide
acatgtttttgtagactagtgcatgcaccggagatctgtaactctccataagcctagccggacggcctccgaaaccatg

SEQ ID NO: 18 - DNA, 80 bp, Artificial Sequence
  Oligonucleotide
aaaaaggtaataattaattgctttagcattcaataattaacaacaaagtcaaaaccttatgataaccgattttcctgagg

SEQ ID NO: 19 - DNA, 80 bp, Artificial Sequence
  Oligonucleotide
aaatatgacgtcaaccgacatgtttttgtagactagtgcatgcaccggagatctgtaactctccataagcctagatgggc

SEQ ID NO: 20 - DNA, 80 bp, Artificial Sequence
  Oligonucleotide
ggtaataattaattgctttagcattcaataattaacaacaaagtcaaaattcctccaacaggcagctctggctaacagtg

SEQ ID NO: 21 - DNA, 80 bp, Artificial Sequence
  Oligonucleotide
atgtttttgtagactagtgcatgcaccggagatctgtaactctccataagcctagccatgttcatgaacaagccctttgg

SEQ ID NO: 22 - DNA, 80 bp, Artificial Sequence
  Oligonucleotide
aggtaataattaattgctttagcattcaataattaacaacaaagtcaaaattcctccaacagtcgaggctgatcagcggg

SEQ ID NO: 23 - DNA, 80 bp, Artificial Sequence
  Oligonucleotide
catgtttttgtagactagtgcatgcaccggagatctgtaactctccataagcctagccatgacacacaacggtggtcgtc

SEQ ID NO: 24 - DNA, 80 bp, Artificial Sequence; identical to SEQ ID NO: 22
  Oligonucleotide

SEQ ID NO: 25 - DNA, 80 bp, Artificial Sequence; identical to SEQ ID NO: 7
  Oligonucleotide

SEQ ID NO: 26 - DNA, 80 bp, Artificial Sequence; identical to SEQ ID NO: 22
  Oligonucleotide

SEQ ID NO: 27 - DNA, 80 bp, Artificial Sequence
  Oligonucleotide
gacatgtttttgtagactagtgcatgcaccggagatctgtaactctccataagcctagccggcaccaaaatcaacgggac

SEQ ID NO: 28 - DNA, 80 bp, Artificial Sequence
  Oligonucleotide
gtaataattaattgctttagcattcaataattaacaacaaagtcaaaattcctccaacagttattaggacaaggctggtg

SEQ ID NO: 29 - DNA, 80 bp, Artificial Sequence
  Oligonucleotide
ttccagaagtagtgaggaggcttttttggaggcctaggcttttgcaaaaagctttgcaaaggcaccaaaatcaacgggac

SEQ ID NO: 30 - DNA, 80 bp, Artificial Sequence
  Oligonucleotide
tgaatgggcttccatagtttgaaagaaaaaccctagcagtactggcaagggagacattccttattaggacaaggctggtg

(19) United States
(12) Patent Application Publication
(10) Pub. No.: US 2010/0179158 A1
(43) Pub. Date: Jul. 15, 2010

(54) INHIBITORS OF CYCLIC AMP PHOSPHODIESTERASES

(76) Inventor: Charles S. Hoffman, Wenham, MA (US)

Correspondence Address: WOLF GREENFIELD & SACKS, P.C., 600 ATLANTIC AVENUE, BOSTON, MA, 02210-2206, US

(21) Appl. No.: 12/596,504

(22) PCT Filed: Apr. 18, 2008
(86) PCT No.: PCT/US08/05003

§ 371 Date: Mar. 4, 2010

Related U.S. Application Data
(60) Provisional application No. 60/925,503, filed on Apr. 20, 2007.

(51) Int. Cl. A61K 31/4985; A61K 31/505; A61K 31/4025; A61K 31/40; A61K 31/381; A61K 31/122; A61K 31/22; A61P 25/00; A61P 25/22; A61P 25/18; A61P 25/16; A61P 25/24; A61P 35/02; A61P 3/04; A61P 3/10; A61P 9/10; A61P 19/10

(52) U.S. Cl. 514/250; 514/274; 514/422; 514/423; 514/443; 514/680; 514/546

(57) ABSTRACT

Recombinant fission yeast cells and methods of using them are described, which provide for identification of chemical and biological inhibitors or activators of a target exogenous phosphodiesterase (PDE). The invention provides, in some aspects, compounds that inhibit cAMP PDE activity and compositions that include such compounds. The invention, in part, also includes methods of using cAMP PDE-inhibiting compounds in the treatment of cAMP PDE-associated diseases and/or disorders.

### RELATED APPLICATIONS

This application claims the benefit of U.S. provisional application 60/925,503, filed Apr. 20, 2007. The entire teachings of the referenced provisional application are incorporated by reference herein in its entirety.

### GOVERNMENT SUPPORT

The invention was made with government support under grant GM46226 and grant GM79662, each awarded by the National Institutes of Health. The United States Government has certain rights in the invention.

### FIELD OF THE INVENTION

The present invention provides methods for treating inflammatory diseases comprising either the administration of a dual phosphodiesterase 7-phosphodiesterase 4 (PDE7-PDE4) inhibitors, or the simultaneous or sequential co-administration of a selective PDE7 inhibitors together with a selective PDE4 inhibitors. The present invention further relates to pharmaceutical compositions containing these inhibitors, and the use of these inhibitors in the treatment of inflammatory diseases.

### BACKGROUND OF THE INVENTION

Phosphodiesterases (PDEs) play an important role in various biological processes by hydrolysing the key second messengers adenosine and guanosine 3′,5′-cyclic monophosphates (cAMP and cGMP respectively) into their corresponding 5′-monophosphate nucleotides. Therefore, inhibition of PDE activity produces an increase of cAMP and cGMP intracellular levels that activate specific protein phosphorylation pathways involved in a variety of functional responses. At least 11 families of PDEs exist, some of which (PDE 3, 4, 7, 8) are specific for cAMP, and others (PDE 5, 6, 9) for cGMP, while other family members have dual specificity (PDE 1, 2, 10, 11). PDEs are expressed in a tissue and cell specific manner, and expression also changes depending on the cell state. For example, resting T lymphocytes express mainly PDE3 and PDE4. However, upon activation, T cells dramatically upregulate PDE7 and appear to rely on this isozyme for regulation of cAMP levels.

Eight isoforms of PDE1 have been identified and are distributed in heart, lung, and kidney tissue, as well as in circulating blood cells and smooth muscle cells. PDE2 is expressed in adrenal gland, heart, lung, liver, and platelets. The PDE3 family of enzymes (four isoforms) are distributed in several tissues including the heart, lung, liver, platelets, adipose tissue, and inflammatory cells. Twenty isoforms of PDE4 exist, and these are expressed in a wide variety of tissues including heart, kidney, brain, liver, lung, the gastrointestinal track and circulating blood and inflammatory cells. PDE5 (three isoforms) is expressed for example in the human corpus cavernosum (vascular) smooth muscle, lung, and platelets. PDE6 is expressed in photoreceptors of the retina. PDE7 has three isoforms and is expressed in skeletal muscle, heart, kidney, brain, pancreas, and T lymphocytes. PDE8 is expressed in testes, eye, liver, skeletal muscle, heart, kidney, ovary, brain, and T lymphocytes. PDE9 with four isoforms is expressed in kidney, liver, lung, brain. PDE10 with two isoforms is expressed in the testes as well as the brain. PDE11 has four isoforms and is expressed in skeletal muscle, prostate, kidney, liver, pituitary and salivary glands, and testes (Boswell-Smith V. et al., 2006, Brit J Pharm 147:S252-57).

The four PDE4 subfamilies are encoded by separate genes (A, B, C, D) that generate a many isoforms through the use of alternative mRNA splicing and distinct promoters. Isoforms generated by the four PDE4 subfamilies are each individually characterized by unique N-terminal regions. They can be divided into long forms, which possess both the Upstream Conserved Region 1 (UCR1) and Upstream Conserved Region (UCR2) regulatory regions, while the short isoforms lack UCR1 and the super-short isoforms lack UCR1 and also have a truncated UCR2.

Two PDE7 genes (PDE7A and PDE7B) have been identified. PDE7A has three isoforms generated by alternate splicing; PDE7A1 restricted mainly to T cells and the brain, PDE7A2 for which mRNA is expressed in a number of cell types including muscle cells, and PDE7A3 found in activated T cells. The PDE7A1 and PDE7A2 isoforms have different sequence at the amino termini. PDE7A3 is similar to PDE7A1 in the amino terminus but has a different carboxy terminal sequence than PDE7A1 and PDE7A2. PDE7B has approximately 70% homology to PDE7A in the enzymatic core.

PDEs are important drug targets. Many PDE-specific inhibitors have been developed and are currently being used or are being evaluated for use, such as KS-505a (PDE1); E1-INA (PDE2); Cilostamide, Enoxamone, Milrinone, Siguazodan (PDE3); Rolipram, Roflumilast, Cilomilast (PDE4); Sildenafil, Zaprinast (PDE5); Dipyridamole (PDE6); BRL-50481 (PDE7), BAY 73-6691 (PDE9) (Boswell-Smith V. et al., 2006, Brit J Pharm 147:S252-57).

PDE2 inhibitors were developed for the treatment of sepsis, and Acute Respiratory Distress Syndrome (ARDS).

PDE3 inhibitors were developed for the treatment of congestive heart failure, airway diseases, and to treat fertility. PDE3 inhibitors have been shown to relax vascular and airway smooth muscle, inhibit platelet aggregation and induce lipolysis.

PDE4 inhibitors were developed for the treatment of inflammatory airways disease, asthma, chronic obstructive pulmonary disease (COPD), allergic rhinitis, psoriasis, rheumatoid arthritis, depression, schizophrenia, Alzheimer's Disease, memory loss, cancer, dermatitis and multiple sclerosis. Inhibition of PDE4 has been associated with an anti-inflammatory response associated with T cells as well as monocytes, macrophages, mast cells, basophils and neutrophils. The majority of PDE4 selective inhibitors reported on to date serve to inhibit PDE4 isoforms from all the four subfamilies with either little or no PDE4 subfamily selectivity, while PDE4A and PDE4B are the actual anti-inflammatory targets.

PDE5 inhibitors were developed for the treatment of erectile dysfunction and impotence, pulmonary hypertension, female sexual dysfunction, cardiovascular disease, premature ejaculation, stroke, leukaemia, and renal failure.

PDE7 inhibitors were developed for the treatment of inflammation. Increasing cAMP levels by selective PDE7 inhibition appears to be a potentially promising approach to specifically block T-cell mediated immune responses.

There are side-effects associated with many PDE inhibitors, which limit their use. PDE1 inhibitors have demonstrated potent vasodilator activity. PDE3 inhibitors have demonstrated potent cardiac inotropic activity. Nausea, emesis and cardiac arrhythmias remain the major obstacles in the development of PDE4 inhibitors, especially caused by inhibition of PDE4D. PDE5 inhibitors affect PDE6 activity in the photoreceptors of the retina and can lead to visual disturbances consisting of altered color perception. There is an unmet medical need to develop effective methods and identify effective PDE inhibitor compounds, including PDE inhibitors that specifically act on individual family members and even on individual isoforms expressed from a single PDE gene, for treatment of immune and inflammatory disorders.

### SUMMARY OF THE INVENTION

Described herein are PDE4 inhibitors (e.g., PDE4A inhibitors, PDE4B inhibitors), PDE7 inhibitors, combination inhibitors (e.g., PDE4A/4B, PDE4/7); methods in which such inhibitors are used, including methods in which an inhibitor is used to treat a condition or disease (e.g., an inflammatory disease, a neurological disease, memory loss, chronic lymphocytic leukemia, osteoporosis, HIV infection, cerebrovascular ischemia); and pharmaceutical compositions comprising at least one PDE4 inhibitor (e.g., PDE4A inhibitor, PDE4B inhibitor), PDE7 inhibitor, PDE4/7 combination inhibitor) and an appropriate carrier. The pharmaceutical composition can optionally additionally comprise at least one additional drug.

PDE inhibitors were identified using methods described herein, such as high throughput drug screens on genetically engineered fission yeast strains that express drug targets (e.g., PDE4A and/or PDE4B, which are anti-inflammatory targets). PDE inhibitors were identified based on their ability to stimulate growth and compounds were identified because they were effective in live cells. In addition, targets used in the assays are full-length proteins (as opposed to simply the catalytic domain) and the assay used included a built-in toxicity test, permeability test and stability test. The inhibitors identified display a very high degree of target specificity. Compounds identified include inhibitors that act on two of three PDE4 family enzymes and inhibitors that act on combinations of PDE4 and PDE7 strains. One example of a compound identified is compound 26, which is an effective PDE4A/4B inhibitor that exhibits limited/essentially no inhibition of PDE4D. Limited inhibition of PDE4D by a PDE inhibitor is desirable, in view of the fact that inhibition of PDE4D causes emesis and cardiac arrhythmias. Subtype specificity was confirmed by means of cAMP assays.

As described herein and as shown in the tables, Applicant has identified compounds that are PDE4A inhibitors; PDE4B inhibitors; PDE4A, 4B inhibitors; PDE7 inhibitors; and PDE4A, 4B and 7 inhibitors. Inhibitors described herein can be used individually (e.g., a PDE4A inhibitor; a PDE4B inhibitor; a PDE7 inhibitor; a combination inhibitor, such as a PDE4A, 4B inhibitor, PDE4/7 inhibitor or a PDE4A, 4B, 7 inhibitor) or in combination with one or more other PDE inhibitor(s) (e.g., PDE4A inhibitor with a PDE4B inhibitor and/or a PDE7 inhibitor) or in combination with another therapeutic agent/drug that is also a PDE inhibitor or another therapeutic agent/drug that is not a PDE inhibitor.

Co-administration of PDE inhibitors, which may be selective for the PDE family, a specific PDE subfamily, or a specific isoform of a PDE-subfamily member, such as a selective PDE4 inhibitor with a selective PDE7 inhibitor, or administration of a dual PDE7-PDE4 inhibitor, can be used to increase therapeutic effectiveness, and/or reduce toxicity and/or side effects (such as nausea) over presently-available approaches. The combined activity of PDE4 and PDE7 or dual PDE7/4 inhibitors may be especially useful in treating a wide variety of immune and inflammatory disorders as an immunosuppressant therapy. PDE7 inhibitors act by inhibiting a very early stage of the T cell activation cascade. PDE4 inhibition decreases the production of the pro-inflammatory cytokines such as Tumor Necrosis Factor alpha, (TNF-α) in monocytes and macrophages, as well as affect granulocytes, such as neutrophils. Dual PDE4/7 inhibitors or co-administration of selective PDE4 and PDE7 inhibitors are expected to be particularly useful in treating disorders that involve one or more inflammatory response alleviated, at lease in part, by PDE4 inhibition (e.g., via decreased mast cell, basophil and neutrophil degranulation and monocyte and macrophage production of pro-inflammatory cytokines such as TNF-α), and/or are alleviated at least in part by PDE7 inhibition (e.g., though decreased T cell activation), e.g., disorders such as rheumatoid arthritis, inflammatory bowel disease (IBD), psoriasis, asthma, chronic obstructive pulmonary disease (COPD), lupus, visceral pain, osteoarthritis, osteoporosis, allergic rhinitis, cancer, acquired immune deficiency syndrome, allergy, fertility diseases, and multiple sclerosis among others. A PDE4-PDE7 inhibitor combination is also expected to have a decreased potential for clinically significant side effects compared to current immunosuppressants.

### BRIEF DESCRIPTION OF THE FIGURES

FIG. 1 shows growth of fission yeast strains carrying mutations in the adenylate cyclase (git2) gene, the PDE (cgs2) gene, or the git1 (a regulator of adenylate cyclase) gene on various growth media. The arrows point to two strains that demonstrate that a reduction in PDE activity can restore 5FOA-resistant growth to either a git2-7 or git1-1 mutant strain. Note that the git2 deletion strain (git2z-1) remains 5FOA-sensitive even when carrying the cgs2-s1 mutation.

FIG. 2 shows β-galactosidase activity resulting from fbp1-lacZ expression as a function of time after removal of cAMP from the growth medium. β-galactosidase activity was measured at the time points indicated after cells were transferred from EMM medium containing 5 mM cAMP to EMM without cAMP.

FIG. 3 shows schematic diagrams of cAMP-regulated growth phenotypes in fission yeast strains expressing the fbp1-ura4 reporter. FIG. 3A is a diagram showing that glucose signaling leads to adenylylcyclase activation and a cAMP signal, which activates PKA to repress fbp1-ura4 transcription. These cells cannot grow in medium lacking uracil (-Ura), but do grow in medium containing 5FOA. FIG. 3B is a diagram showing that cells carrying mutations in genes required for glucose signaling have reduced adenylylcyclase activity to lower cAMP levels. This results in low PKA activity and a failure to repress fbp1-ura4 transcription. These cells grow in medium lacking uracil (-Ura), but do not grow in medium containing 5FOA. FIG. 3C is a diagram showing a screen for PDE activators carried out by taking a strain such as the one in panel A and screening for compounds that enhance growth in medium lacking uracil. The compounds identified include ones that stimulate PDE activity to lower cAMP levels. FIG. 3D is a diagram showing a screen for PDE inhibitors carried out by taking a strain such as the one in FIG. 3B and screening for compounds that enhance growth in 5FOA medium. The compounds identified include ones that inhibit PDE activity to raise cAMP levels.

FIG. 4 is a graph showing that deletion of pap1⁺ enhances rolipram-mediatedfbp1-lacZ repression. β-galactosidase activity from two independent exponential phase cultures was determined in pap1⁺ (light gray bars) and pap1 Δ(dark gray bars) gpa2⁻ mutant strains grown in EMM complete medium containing various concentrations of rolipram as indicated, while receiving identical volumes of DMSO (vehicle). Values are plotted as a percent of the vehicle-treated cultures that did not receive rolipram. The ratio of fold-inhibition in the pap1Δ strain versus the pap1⁺ strain is shown for each concentration of rolipram.

FIG. 5 shows graphs demonstrating that PDE inhibitors alter cAMP levels in yeast strains. FIG. 5A shows results when cAMP levels were measured in exponential phase cells immediately prior to 200 μM drug addition (rolipram for strains CHP1085 (PDE4A) and CHP1114 (PDE4B), and EHNA for strain LWP371 (PDE2A)), and 10, 30, 60, and 120 minutes after drug addition. Values represent the average and SD of two or three independent experiments. FIG. 5B shows results when cAMP levels were measured 60 minutes after addition of either vehicle (DMSO), 20 μM drug, or 200 μM drug as indicated. The strains used are as in FIG. 5A, together with strain CHP1141 (PDE8A). Values represent the average and SD of two or three independent experiments.

FIG. 6 is a graph of results of assessment of compound 26, which shows that compound is an effective PDE4A/4B inhibitor.

FIG. 7 is a graph of results of assessment of compound 26, which show that compound 26 exhibits little or no inhibition of PDE4D.

FIG. 8 shows results of cAMP assays that confirm subtype-specificity.

FIG. 9 is a graph showing results of assessment of effects of group 30 series compounds on PDE7A. Horizontal axis, compound concentration; vertical axis, O.D.

FIG. 10 is a graph showing results of assessment of group 26 compounds. Shown are growth curves for human PDE4A1. Horizontal axis, compound concentration; veritcal axis, compound concentration.

Also included are Tables 1 through 11.

### DETAILED DESCRIPTION OF THE INVENTION

Described herein are methods for treating a wide variety of immune and inflammatory disorders using PDE4 inhibitor(s), PDE7 inhibitor(s); a combination of PDE4 inhibitors and PDE7 inhibitors, or dual PDE4A, 4B, or dual PDE7/4 inhibitors, which may be selective for the PDE family, a specific PDE subfamily, or a specific isoform of a PDE-subfamily member. Also described are compounds and compositions that include at least one PDE4 inhibitor (e.g., a PDE4A inhibitor, a PDE4B inhibitor); at least one PDE7 inhibitor, at least one combination inhibitor (e.g., PDE4A/4B inhibitor, PDE4/7 inhibitor) or a combination of two or more such inhibitors. Such compositions may also include a pharmaceutically acceptable carrier. When administered to an individual, the compounds inhibit PDE4 and/or PDE7 activity in vivo and are useful for treating immune and inflammatory disorders. The selective PDE4 or PDE7 inhibitor compounds described herein, used alone or in combination, and dual PDE4/7 inhibitors may be used. Combinations (e.g., combinations of two or more PDE4 inhibitors (e.g., PDE4A inhibitor and PDE4B inhibitor); combinations of one or more PDE4 inhibitor with a PDE7 inhibitor) may be more effective than either a selective PDE4 inhibitor or a selective PDE7 inhibitor administered alone in the treatment of disease, through additive or synergistic activity resulting from the combined inhibition of PDE4 and PDE7. Expression of PDE7A, for example, increases when PDE4 is inhibited.

Described herein are compounds that exhibit low toxicity against biological organisms in vitro. In some embodiments the compounds exhibit the ability to permeate biological organisms in vitro, e.g., to cross a biological membrane. In some embodiments the compounds exhibit high bio-stability in biological organisms in vitro, e.g., are not rapidly degraded or are active for an extended period.

There are numerous compounds described herein. They are grouped into Groups I-VI, as shown below. In certain embodiments the compounds are selected from compounds of formula (I) (Group I).

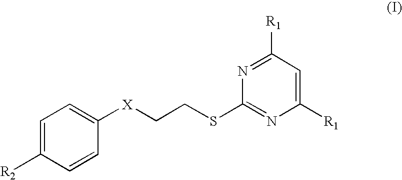

wherein

### X is SO, or SO₂,

R1 is H, or alkyl,
R2 is alkyl, or halogen.

In specific embodiments, R1 is Me. In other specific embodiments R1 is F. In certain embodiments R2 is t-Bu. In specific embodiments, R1 is methyl. In more specific embodiments, the compounds are selected from:

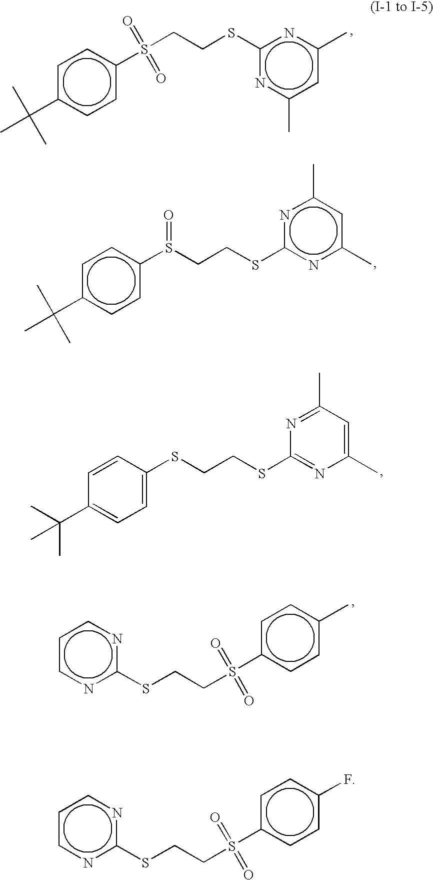

In certain embodiments the compounds of the invention can also be selected from compounds of formula (II) (Group II):

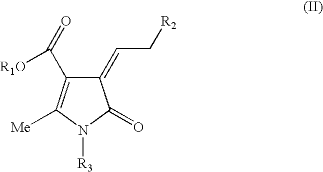

wherein
R1 is alkyl,
R2 is aryl or heteroaryl,
R3 is alkyl, aryl, cycloakyl, or alkylaryl.

In specific embodiments, R1 is methyl. In certain embodiments R2 is furanyl or thiophenyl. In other specific embodiments, R2 is substituted phenyl or benzyl. In preferred embodiments, R3 is iso-butyl. In more specific embodiments, the compounds are selected

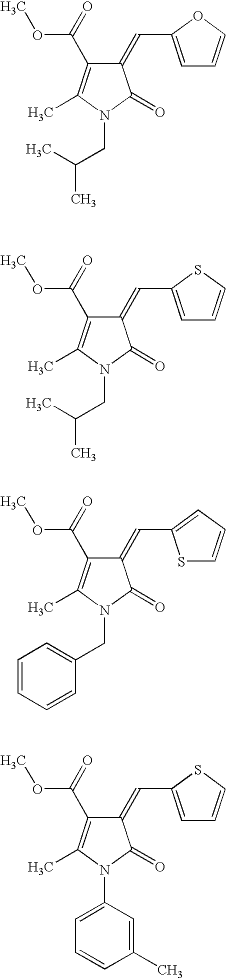

from:

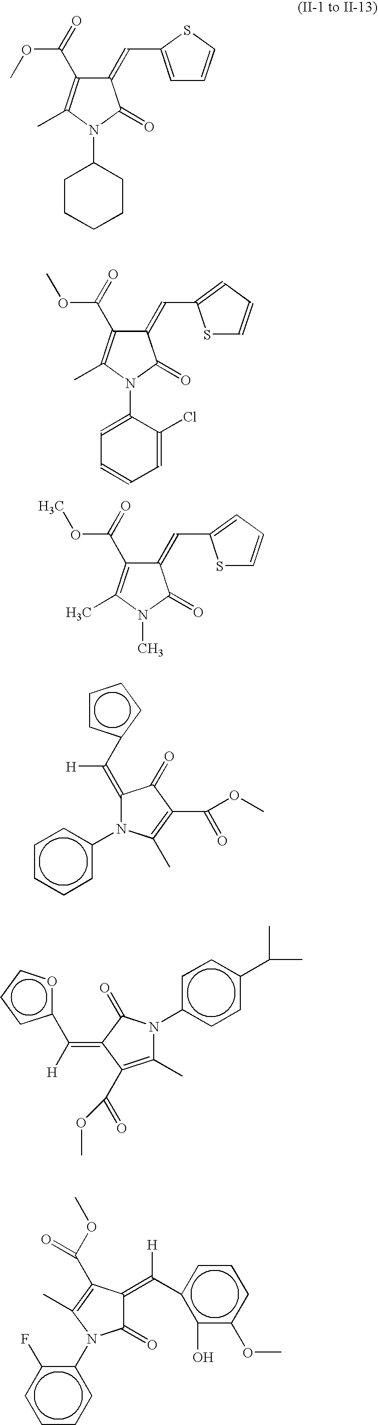

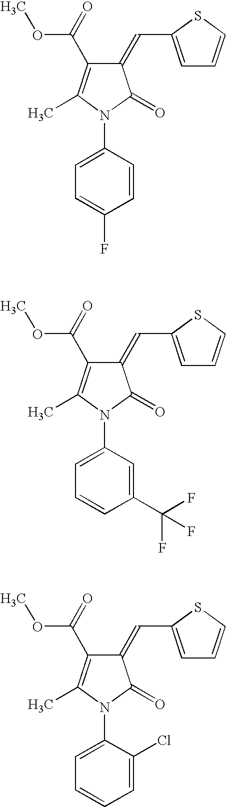

In certain embodiments the invention discloses compounds of formula III (Group III):

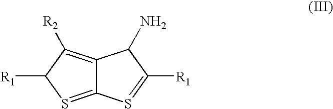

wherein
R1 is nitrile, or alkylcarboxylate,
R2 is alkyl, aryl, or heteroaryl.

In specific embodiments, R1 is nitrile or methylcarboxylate. In certain embodiments, R2 is a five membered heteroaryl. In more specific embodiments, R2 is furanyl, or thienyl. In other embodiments, R2 is a six membered aryl. In more specific embodiments, R2 is substituted phenyl.

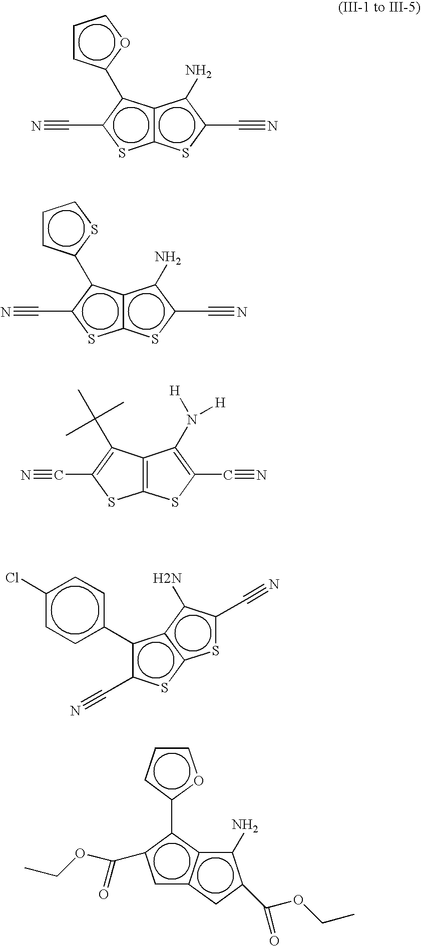

In certain embodiments the compounds of the invention can also have formula IV (Group IV):

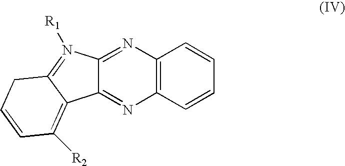

wherein
R1 is alkyl, alkenyl, or alkylcarboxylic acid,
R2 is halogen.

In certain embodiments R1 is butyl. In other embodiments R1 is terminal alkenyl. In more specific embodiments R1 is allyl, or vinyl. In other embodiments, R1 is C₁₋₄alkyl. In specific embodiments R1 is methylcarboxylicacid. In certain embodiments R2 is Cl, or Br. In more specific embodiments, the compounds are selected from:

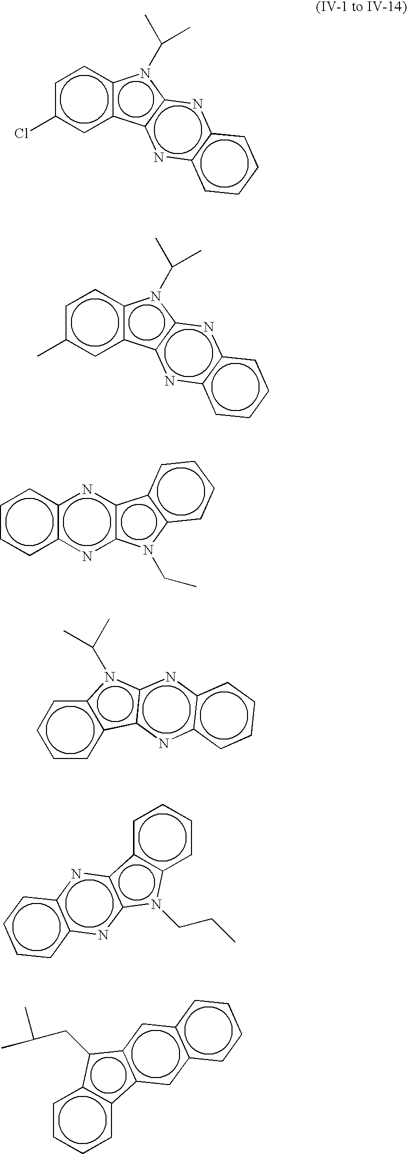

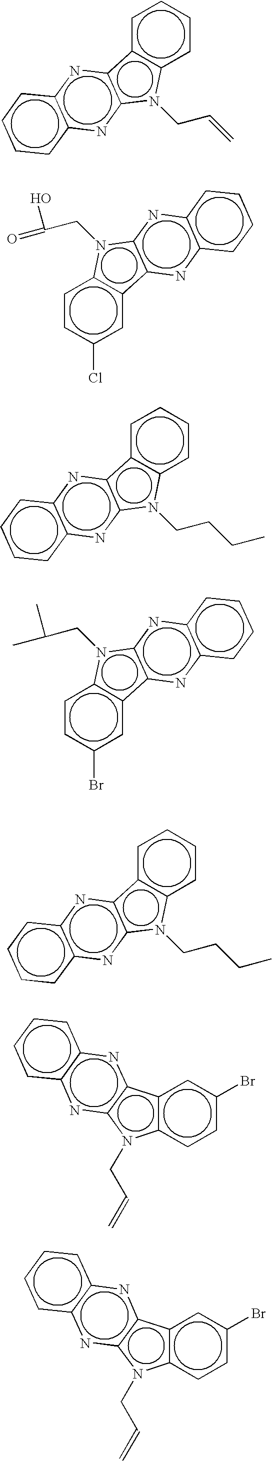

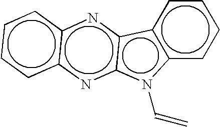

In certain embodiments the some compounds of the invention are compounds of formula V (Group V):

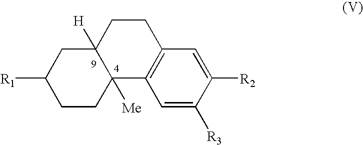

wherein
R1 is CO, or alkylalcohol,
R2 is alkyl,
R3 is alkoxy,
and the C4 and C9 stereocenters are independently (R) or (S).

In certain embodiments R1 is carbonyl, or 2-methylpropan-1-ol. In specific embodiments R2 is methyl. In certain embodiments, R3 is methoxy. In more specific embodiments the compounds are selected from:

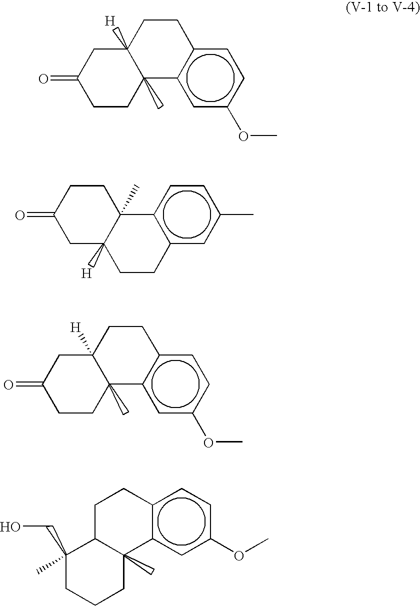

In certain embodiments the compounds of the invention are compounds of formula VI:

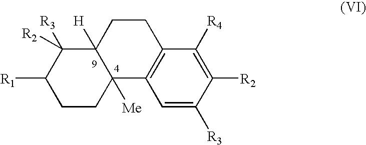

wherein
R1 is hydrogen, hydroxyl, carbonyl, or alkylalcohol,
R2 and R3 are independently selected from hydrogen, alkyl, alkylcarboxylate, or carboxylic acid,
R4 is hydrogen, or alkyl,
R5 is hydrogen, alkyl, hydroxyl, or acetate,
R6 is hydrogen, or alkoxy,
and the C4 and C9 stereocenters are independently (R) or (S).

In certain embodiments R1 is 2-methylpropan-1-ol. In specific embodiments R2 is methyl. In certain embodiments, R2 is methylcarboxylate. In specific embodiments R2 and R3 are both methyl. In other embodiments, R2 is methyl, and R3 is methylcarboxylate. In specific embodiments R4 is iso-propyl. In specific embodiments, R5 is methyl. In certain embodiments, R6 is methoxy. In more specific embodiments the compounds are selected from:

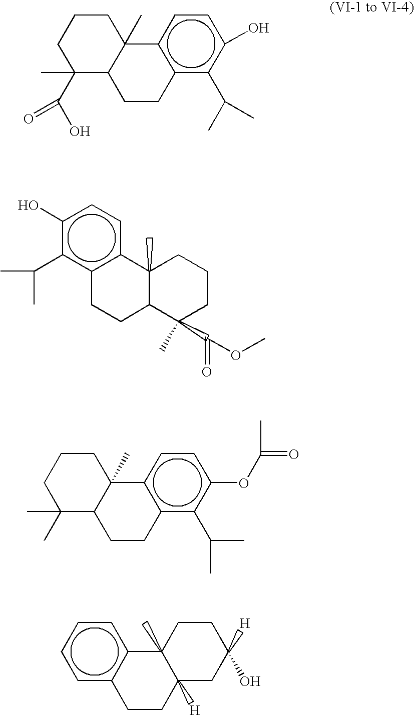

These are referred to as Group VI.

As used herein, the terms “alkyl”, “alkenyl” and the prefix “alk-” are inclusive of both straight chain and branched chain groups and of cyclic groups, i.e. cycloalkyl and cycloalkenyl. Unless otherwise specified, these groups contain from 1 to 20 carbon atoms, with alkenyl groups containing from 2 to 20 carbon atoms. Preferred groups have a total of up to 10 carbon atoms. Cyclic groups can be monocyclic or polycyclic and preferably have from 3 to 10 ring carbon atoms. Exemplary cyclic groups include cyclopropyl, cyclopentyl, cyclohexyl, cyclopropylmethyl, adamantly, norbornane, and norbornee. This is also true of groups that include the prefix “alkyl-”, such as alkylcarboxylic acid, alkyl alcohol, alkylcarboxylate, alkylaryl, and the like. Examples of suitable alkylcarboxylic acid groups are methylcarboxylic acid, ethylcarboxylic acid, and the like. Examples of suitable alkylacohols are methylalcohol, ethylalcohol, isopropylalcohol, 2-methylpropan-1-ol, and the like. Examples of suitable alkylcarboxylates are methylcarboxylate, ethylcarboxylate, and the like. Examples of suitable alkyl aryl groups are benzyl, phenylpropyl, and the like.

The term “aryl” as used herein includes carbocyclic aromatic rings or ring systems. Examples of aryl groups include phenyl, naphthyl, biphenyl, fluorenyl and indenyl. The term “heteroaryl” includes aromatic rings or ring systems that contain at least one ring hetero atom (e.g., O, S, N). Suitable heteroaryl groups include furyl, thienyl, pyridyl, quinolinyl, isoquinolinyl, indolyl, isoindolyl, thazolyl, pyrrolyl, tetrazolyl, imidazolyl, pyrazolyl, oxazolyl, thiazolyl, benzofuranyl, benzothiophenyl, carbazolyl, benzoxazolyl, pyrimidinyl, benzimidazolyl, quinoxalinyl, benzothiazolyl, naphthyridinyl, isoxazolyl, isothiazolyl, purinyl, quinazolinyl, and so on.

The aryl, and heteroaryl groups can be unsubstituted or substituted by one or more substituents independently selected from the group consisting of alkyl, alkoxy, methylenedioxy, ethylenedioxy, alkylthio, haloalkyl, haoalkoxy, haloalkylthio, halogen, nitro, hydroxy, mercapto, cyano, carboxy, formyl, aryl, aryloxy, arylthio, arylalkoxy, arylalkylthio, heteroaryl, heteroaryloxy, heteroarylalkoxy, heteroarylalkylthio, amino, alkylamino, dialkylamino, heterocyclyl, heterocycloalkyl, alkylcarbonyl, alkenylcarbonyl, alkoxycarbonyl, haloalkylcarbonyl, haloalkoxycarbonyl, alkylthiocarbonyl, arylcarbonyl, heteroarylcarbonyl, aryloxycarbonyl, heteroaryloxycarbonyl, arylthiocarbonyl, heteroarylthiocarbonyl, alkanoyloxy, alkanoylthio, alkanoylamino, arylcarbonyloxy, arylcarbonylhio, alkylaminosulfonyl, alkylsulfonyl, arylsulfonyl, heteroarylsulfonyl, aryldiazinyl, alkylsulfonylamino, arylsulfonylamino, arylalkylsulfonylamino, alkylcarbonylamino, alkenylcarbonylamino, arylcarbonylamino, arylalkylcarbonylamino, arylcarbonylaminoalkyl, heteroarylcarbonylatnino, heteroarylalkycarbonylamino, alkylsulfonylamino, alkenylsulfonylamino, arylsulfonylamino, arylalkylsulfonylamino, heteroarylsulfonylamino, heteroarylalkylsulfonylamino, alkylaminocarbonylamino, alkenylaminocarbonylamino, arylaminocarbonylamino, arylalkylaminocarbonylamino, heteroarylaminocarbonylamino, heteroarylalkylaminocarbonylamino and, in the case of heterocyclyl, oxo. If other groups are described as being “substituted” or “optionally substituted,” then those groups can also be substituted by one or more of the above enumerated substituents.

According to other aspects of the invention, methods for treating a PDE-associated disease or condition in an individual are provided. The methods include administering to an individual in need of such treatment an effective amount of a compound or composition (e.g., pharmaceutical composition) described herein to treat the PDE-associated disease or condition in the individual. The individual can be a human or other mammal. In some embodiments the PDE-inhibiting compound, which may be a combination of a PDE4 inhibitor, such as a selective PDE4 inhibitor, and a PDE7 inhibitor, such as a selective PDE7 inhibitor, or a combination/dual PDE4/7 inhibitor, is linked to a targeting molecule. In some embodiments the PDE-inhibiting compound is administered prophylactically to a person at risk of developing a PDE-associated disease or disorder.

PDE4 inhibitors, such as selective PDE4 inhibitors (PDE4A, PDE4B) and/or PDE7 inhibitor, such as selective PDE7 inhibitors, and/or dual PDE4-PDE7 inhibitor compounds or pharmaceutical compositions comprising one or more inhibitor and methods described herein, are useful in the treatment (including prevention, partial alleviation or cure) of disorders, which include, but are not limited to, disorders such as: transplant rejection (such as organ transplant, acute transplant, xenotransplant or heterograft or homograft such as is employed in burn treatment); protection from ischemic or reperfusion injury such as ischemic or reperfusion injury incurred during organ transplantation, myocardial infarction, stroke or other causes; transplantation tolerance induction; arthritis (such as rheumatoid arthritis, psoriatic arthritis or osteoarthritis); multiple sclerosis; respiratory and pulmonary diseases including but not limited to asthma, exercise induced asthma, chronic obstructive pulmonary disease (COPD), emphysema, bronchitis, and acute respiratory distress syndrome (ARDS); inflammatory bowel disease, including ulcerative colitis and Crohn's disease; lupus (systemic lupus erythematosis); graft vs. host disease; T-cell mediated hypersensitivity diseases, including contact hypersensitivity, delayed-type hypersensitivity, and gluten-sensitive enteropathy (Celiac disease); psoriasis; contact dermatitis (including that due to poison ivy); Hashimoto's thyroiditis; Sjogren's syndrome; Autoimmune Hyperthyroidism, such as Graves' Disease; Addison's disease (autoimmune disease of the adrenal glands); Autoimmune polyglandular disease (also known as autoimmune polyglandular syndrome); autoimmune alopecia; pernicious anemia; vitiligo; autoimmune hypopituatarism; Guillain-Barre syndrome; other autoimmune diseases; glomerulonephritis; serum sickness; uticaria; allergic diseases such as respiratory allergies (e.g., asthma, hayfever, allergic rhinitis) or skin allergies; scleracierma; mycosis fungoides; acute inflammatory and respiratory responses (such as acute respiratory distress syndrome and ishchemia/reperfusion injury); dermatomyositis; alopecia greata; chronic actinic dermatitis; eczema; Behcet's disease; Pustulosis palmoplanteris; Pyoderma gangrenum; Sezary's syndrome; atopic dermatitis; systemic schlerosis; and morphea, and cancer.

Other examples of diseases and disorders associated with cAMP PDE activity and/or abnormal cAMP or cGMP levels include, but are not limited to neurodegenerative disorders, penile erectile dysfunction, anxiety, depression, Alzheimer's disease, Parkinson's disease, Huntington's disease, schizophrenia, psychosis, sepsis, renal disease, memory loss, chronic lymphocytic leukemia, prostate cancer, thyroid disease, male hypogonadism, cardiac disease, diabetes, obesity, osteoporosis, and cystic fibrosis.

### DEFINITIONS

A “cyclic AMP phosphodiesterase” or “cAMP PDE” as used herein refers to an enzyme from any biological source which hydrolyzes the substrate 3′,5′-cyclic adenosine monophosphate to yield 5′-adenosine monophosphate. A cAMP PDE may also hydrolyze other substrates, such as 3′,5′-cyclic guanosine monophosphate (cGMP); the enzyme need not have a complete or even a preferential specificity for cAMP. A cAMP PDE of the presently disclosed embodiments can also be a fragment, a mutant, or a post-translationally modified variant of a naturally occurring PDE.

Examples of cAMP PDEs that specifically hydrolyze the substrate 3′,5′-cyclic adenosine monophosphate to yield 5′-adenosine monophosphate and do not hydrolyze 3′,5′-cyclic guanosine monophosphate include, PDE4A, PDE4B, PDE4C, PDE4D, PDE7A, PDE7B, PDE8A, and PDE8B. Examples of cAMP PDEs that hydrolyze the substrate 3′,5′-cyclic adenosine monophosphate to yield 5′-adenosine monophosphate and also hydrolyze 3′,5′-cyclic guanosine monophosphate to yield 5′-guanosine monophosphate include: PDE1A, PDE1B, PDE1C, PDE2A, PDE3A, PDE3B, PDE10A, or PDE11A. It will be understood by those of ordinary skill in the art that the PDEs useful in cells and assays of the invention include PDEs listed herein, and also include splice variants of the PDE families. The identities and sequences of splice variants of PDE families are known and/or are readily identifiable by those of ordinary skill in the art. For example, although not intended to be limiting, PDE4A1 and PDE4A5 are both splice variants of PDE4A, thus the listing of PDE4A herein is understood to include PDE4A1 and PDE4A5. Thus, the invention encompasses the use of splice variants of the PDE families provided herein in cells and assays methods of the invention. Those of ordinary skill in the art will understand that an exogenous PDE that may be included in a yeast cell of the invention can be from any PDE family listed herein, and that the PDE family members include PDEs provided herein and splice variants thereof.

A “recombinant yeast cell” or “recombinant fission yeast cell” as used herein is a yeast cell into which a foreign nucleic acid (not originating from or identical to a nucleic acid of the same species) has been incorporated by any available technique of molecular biology. Such a recombinant yeast cell may be representative of a larger number of cells, such as a genetic strain, and any cell or method described or claimed herein in the singular is understood to also encompass the plural. A recombinant yeast cell can be, for example, a yeast cell that has been transformed with the DNA encoding a foreign, e.g. exogenous, cAMP PDE. A recombinant yeast cell which is “lacking endogenous PDE” is one that expresses little or no PDE, i.e., 5%, 2%, 1%, or less of the PDE enzyme activity found in a wild type yeast cell of the same species, unless an exogenous gene encoding a PDE has been added to the cell. An “exogenous PDE” is a PDE whose amino acid sequence is different from a PDE of the yeast species into which it is introduced. Exogenous PDE genes for use in the presently disclosed embodiments, include, for example, any human PDE, any mammalian PDE, non-mammalian PDE, and/or any gene from an organism that encodes a protein with PDE activity.

A “fission yeast” or “fission yeast cell” as used herein refers to a unicellular fungus that divides by medial fission. The fission yeast of the presently disclosed embodiments is a yeast of the genus Schizosaccharomyces; a preferred fission yeast is the species Schizosaccharomyces pombe, including any strain derived therefrom. As used herein the terms, “derived from” or “derived therefrom” mean that a yeast strain has been specifically engineered from an original strain. For example, though not intended to be limiting, a cell that includes a cAMP PDE gene and is derived from Schizosaccharomyces pombe (S. pombe), is a cell originated from an S. pombe cell and the S. pombe cell was specifically engineered to include the cAMP PDE gene.

A “reporter construct” as used herein refers to a nucleic acid construct that can be stably transformed into a fission yeast cell, and generally comprises one or more reporter genes under transcriptional control of a promoter. The one or more reporter genes of a reporter construct serve to provide a “detectable signal” upon expression. The detectable signal is any measurable parameter which evidences, in a qualitative or quantitative way, the expression of the reporter gene product in the host fission yeast cell. Examples of detectable signals of reporter genes suitable for use in the presently disclosed embodiments include protein fluorescence (e.g., the fluorescence emission of green fluorescent protein (GFP), red fluorescent protein (RFP), or yellow fluorescent protein (YFP)) and enzyme activity (e.g., β-galactosidase activity), which are well known in the art. Further suitable detectable signals include, but are not limited to, the turbidity, light scattering, or optical density of a cell suspension (indicative of cell growth resulting from reporter activity), or growth in a particular culture medium (e.g., growth in “high glucose” fission yeast culture medium (glucose concentration of at least 3% wt/vol, preferably about 8% wt/vol), or growth in the presence of 5-fluoro-orotic acid (5FOA) or in the absence of uracil). Moreover, activities of fission yeast cells which are dependent on cAMP levels can be used as a detectable signal to monitor PDE activity. Examples include conjugation and sporulation, which require low cAMP levels to occur; higher levels due to PDE inhibition or the absence of a PDE gene would inhibit such processes.

In methods and cells of the invention, a detectable signal may be compared to a control detectable signal. As used herein, a “control detectable signal” is a signal detected in a cell or cell population that is substantially equivalent to the cell or population under equivalent assay conditions, except that a parameter being tested for its effect of PDE activity, for example, a modulating compound (e.g., a test compound), or a cDNA library, is not present in the assay conditions of the control cell or population. A non-limiting example is an assay to identify a modulator of PDE, recombinant yeast cells of the invention may be contacted with a test compound and a detectable signal measured in the cells. A control detectable signal may be the detectable signal generated in a control population of cells that is substantially equivalent (e.g., recombinant with the same genetic characteristics as the test cells) and under essentially the same assay conditions, but the control cells are not contacted with the test compound. Thus, by comparing a detectable signal in cells contacted with the test compound to the detectable signal in cells not contacted with the test compound, differences in the responses of the two populations can be determined. Differences between the test and control, (increases or deceases), are indicative of a modulatory effect of the test compound on the PDE activity. A control detectable signal may be an established value based on previous tests, or may be a signal detected in assays run in parallel with a test assay. Those of ordinary skill in the art will understand and will be able to establish control values, use control values, and compare test with control values using only routine methods.

The promoter determines the transcription of the reporter gene and therefore determines the condition in the cell which is reported as a detectable signal. The promoter can be derived from fission yeast or from another organism. A promoter controls expression of a gene if it is “operably linked” to the gene, which requires that the promoter sequence be situated upstream of the start codon and the open reading frame of the nucleic acid that encodes the reporter protein. In some embodiments, the promoter is “constitutive,” meaning that the gene it controls is continuously expressed. Other promoters provide expression of the gene only when induced by an inducer or certain cell conditions, e.g., low glucose concentration. Promoters suitable for use in the presently disclosed embodiments include, but are not limited to, a constitutive promoter, a PDE promoter, a fission yeast fbp1 promoter, a viral SV40 promoter, and a fission yeast his7 promoter.

The readout for PDE activity is a detectable signal which is sensitive to a change in intracellular cAMP concentration. The terms “cAMP concentration” and “cAMP level” are used interchangeably herein. A level or a concentration of cAMP in a cell can be expressed either in true concentration units (e.g., μmoles per liter) or in terms of an amount of cAMP per mg of cell protein (e.g., pmol cAMP per mg cell protein); a measurement of cAMP amount on a protein basis can be converted to true concentration units by dividing by cell volume (e.g., in μL, per mg protein). In one embodiment, sensitivity of the reporter construct to cAMP is provided through the use of an fbp1 promoter, which is repressed by cAMP-dependent protein kinase (PKA) when cAMP levels rise above approximately 3.5 pmol/mg protein. Other promoters which result in cAMP-dependent reporter gene expression can also be used, such as a git3 or an AC (adenylate cyclase) promoter. As used herein, the phrase “a change in intracellular cAMP concentration” refers to any change in cAMP which produces a detectable signal as a result of reporter gene expression. The “steady-state cAMP concentration” is the concentration of cAMP in a cell prior to the addition of a candidate inhibitor or activator of PDE. Thus, the steady-state cAMP concentration of a given cell or strain can vary depending upon the nature of the experiment (type of culture medium, concentration of glucose, and genetic background). Cyclic AMP levels can be determined by radioimmunoassay¹, ELISA, or by another method known in the art.

As used herein, the term “5FOA resistant growth” or “growth in the presence of 5FOA” refers to the ability of a fission yeast cell that possesses an fbp1-ura4 fusion reporter gene to grow in the presence of about 0.2 to 1.0 gram/liter, preferably 0.4 gram/liter, 5FOA. Such growth requires a low level of Ura4 activity, which results from a high level of cAMP (e.g., more than 3.5 pmol/mg protein), and corresponds to strong inhibition of PDE. Thus, the greater the amount of 5FOA resistant growth by a fission yeast that possesses an fbp1-ura4 fusion reporter gene, the greater is the extent of PDE inhibition. The amount of growth can be determined after any time interval of exposure to a candidate inhibitor or activator, such that a significant change (e.g., in number of cells, density of cells, cell protein, optical density, light scattering, turbidity, or reporter gene fluorescence) can be experimentally determined. In some embodiments, the amount of growth is determined at about 16 to 24 or about 24 to 48 hours or more following addition of the candidate inhibitor or activator.

“Growth in the absence of uracil” as used herein refers to growth of a fission yeast cell that possesses an fbp1-ura4 fusion reporter gene when cAMP levels are low due to a high PDE activity. Low cAMP levels do not support repression of the fbp1-ura4 reporter construct, such that Ura4 activity is high and cell growth is less dependent on uracil in the medium.

A fission yeast cell that “lacks endogenous ura4 activity” is a cell that expresses little or no ura4 gene product (OMP decarboxylase) from the ura4+ genetic locus, e.g., a cell whose OMP decarboxylase activity is 5%, 2%, 1%, or less compared to a wild type fission yeast cell. A “chemical modulator” of PDE as used herein is a small molecule modulator, i.e., any chemical of less than about 2500 daltons molecular weight which alters the rate of a PDE reaction by at least 5%. A chemical modulator may be a cAMP PDE inhibitor or may be a cAMP PDE activator. A cAMP PDE inhibitor is a modulator that reduces the rate of a PDE reaction by at least 5% and a cAMP PDE activator is a modulator increases the rate of a PDE reaction by at least 5%.

A “biological modulator” is a polypeptide, protein, or nucleic acid molecule that alters the rate of a PDE reaction and/or the affinity associated with a PDE enzyme and substrate of a PDE reaction by at least 5%. A biological modulator may be a cAMP PDE inhibitor or may be a cAMP PDE activator. As used herein a biological inhibitor is a polypeptide, protein, or nucleic acid molecule that decreases the rate of a PDE reaction by at least 5%. As used herein a biological activator is a polypeptide, protein, or nucleic acid molecule that increases the rate of a PDE reaction or the affinity associated with a PDE enzyme and substrate of a PDE reaction by at least 5%. Although use of control fission yeast strains in screening assays to identify chemical and biological modulators of PDE can reduce the number of false positives, i.e., some test compounds or gene products identified as inhibitors of PDE might act on cAMP levels through another mechanism or may alter reporter expression through a cAMP-independent manner. Thus, modulators identified in the screening methods of the invention may be considered as candidate modulators of PDE and their function as modulators may be verified using additional screening and testing methods. The terms “modulator” and “candidate modulator” are used interchangeably herein. A substance identified as a candidate modulator of PDE using a fission yeast screen of the invention can be subjected to further testing, e.g., using purified cAMP PDE enzyme in an in vitro assay to investigate the mechanism of action of the candidate modulator and to further explore its suitability as a modulator of PDE in a clinical setting. Thus, suitability of a PDE inhibitor or PDE activator identified using methods and/or recombinant cells of the invention may be further tested for usefulness in therapeutic methods and compositions.

### DESCRIPTION

Fission yeast cells can be genetically modified and used as a screening tool to identify inhibitors and activators of PDE. Fission yeast contain only a single PDE gene. If that gene is replaced by a target PDE gene from an exogenous source, and if the appropriate reporter construct or constructs are introduced, the recombinant yeast cells can provide a rapid readout of their intracellular cAMP concentration, which is a measure of PDE activity. Further, the genetic background of the fission yeast cells can be selected to enhance the sensitivity of detecting changes in cAMP level by altering PDE activity. The cells of the presently disclosed embodiments can be further modified by transformation with a cDNA library from a desired cell or tissue source, thereby allowing identification of biological inhibitors and activators of PDE that can be used as novel targets in high throughput drug screens to identify compounds that alter cAMP metabolism.

Recombinant yeast strains have been prepared in which the S. pombe PDE gene was replaced with a target cAMP PDE gene. Such recombinant yeast strains can be used to screen for chemical or biological modulators of the target cAMP PDE activity. Recombinant yeast strains have been prepared using standard yeast manipulations of the genomic DNA to replace the yeast cgs2⁺ gene with that of a mammalian or pathogen cAMP PDE gene. In some embodiments, the cgs2⁺ gene was initially replaced with the ura4⁺ gene. Next, the target cAMP PDE gene was amplified by PCR using oligonucleotides that possess homology to the cgs2 locus. Cells in which this PCR product has replaced the ura4⁺ gene at cgs2 were selected for on 5FOA-containing plates and confirmed by PCR analysis.

The ura4 gene encodes OMP decarboxylase, which is required for uracil biosynthesis and for sensitivity to the pyrimidine analog 5-fluoro-orotic acid (5FOA). Thus, the fbp1-ura4 fusion may be used as either a selectable or a counterselectable marker, making it extremely useful in genetic screens for mutations or clones that increase or decrease fbp1 transcription. The lacZ gene encodes β-galactosidase, which allows its use in sensitive and rapid assays of expression from the fbp1 promoter that are consistent with direct examination of fbp1⁺ mRNA levels. The fbp1-lacZ fusion disrupts ura4 so that all Ura4 activity in these cells comes from the fbp1-ura4 fusion. Strains carrying these fusions were assessed for their ability to regulate fbp1 transcription. Strains that glucose-repress fbp1-ura4 transcription cannot form single colonies on a glucose-rich medium lacking uracil, but grow on a glucose-rich medium containing 5FOA. Strains that fail to glucose-repress fbp1-ura4 form Ura⁺ colonies on a glucose-rich medium lacking uracil. To generalize, strains that are Ura and 5FOA-senstive have reduced cAMP levels (either basal or glucose-stimulated) as compared with wild type strains, which are Ura⁻ and 5FOA-resistant.

Recombinant yeast strains of the invention may be used in high-throughput screening for cAMP PDE inhibitors by looking for compounds that confer 5FOA-resistant growth. Conversely, cAMP PDE activators can be identified using the strains and are identified as compounds that confer Ura⁺ growth in strains grown in the presence of enough cAMP to normally prevent growth in SC-ura or EMM-ura medium. In addition, a mammalian cDNA library, such as a human cDNA library, constructed in a fission yeast plasmid expression vector is used to screen for biological modulators of the target PDE. Such modulators are the target of subsequent drug screens and may represent an entirely novel drug target. The advantage of this class of drug target is that it may be expressed in a subset of tissues while the PDE may be expressed in a wider range of cell types. As such, targeting the modulator may limit the effect on PDE activity to the desired cells and reduce side effects relative to drugs that directly target the PDE in all cells in which it is expressed. For example, PDE4 inhibitors produce an emetic response. This response may be due to the inhibition of a particular PDE4 enzyme in the brain. Therefore, PDE4 inhibitors that are specific to either individual PDE4 genes (A, B, C, or D) or even to specific splice variants (4A5, but not 4A1) may be therapeutically useful without producing an emetic response. This, specific inhibitors to PDE4 may be advantageously used for preparing a cAMP PDE modulator as a therapeutic that has minimal negative side-effect.

cAMP Signaling And fbp1 Transcriptional Regulation In Fission Yeast

Both the fission yeast Schizosaccharomyces pombe and the budding yeast Saccharomyces cerevisiae produce cAMP signals in response to glucose detection²⁻⁸. In both yeasts, the increase in cAMP levels is due to the activation of adenylate cyclase, while feedback regulation to limit the cAMP response is, in part, a function of PDE activity⁹⁻¹¹. Studies from a number of labs working in both yeasts have shown that the two signaling pathways share many features; however many important distinctions can be made as well. Most importantly, the S. pombe pathway appears to have a single input in which glucose detection is carried out by the Git3 GPCR that then activates the Gpa2 Gα of the Gpa2-Git5-Git11 heterotrimeric G protein¹²⁻¹⁶. In contrast, the cAMP response in budding yeast involves both the GPCR Gpr1 and the Gpa2 Gα, and a pair of Ras proteins along with the Cdc25 guanine nucleotide exchange factor. In addition, an internal glucose signaling mechanism involving glucose-6-phosphate formation is required for S. cerevisiae cAMP signaling⁸. Thus, the S. pombe cAMP signaling pathway appears to be significantly less complex than that of S. cerevisiae.

Most of the genes that act in the S. pombe cAMP pathway were identified by mutations that inhibit glucose repression of transcription of the fbp1 gene that encodes the gluconeogenic enzyme fructose-1,6-bisphosphate¹⁷. The presently disclosed embodiments employ fbp1-driven reporters that allow for the identification of mutations that alter cAMP levels in the cell. Along with genes required for generating a cAMP signal, which activates PKA, negative regulators of PKA were identified by mutations that suppress the temperature-sensitive growth of a pat1-112 mutant strain¹⁸. The cgs1 gene encodes the regulatory subunit of PKA, while the cgs2 gene encodes the only PDE in S. pombe. Using the fbp1-driven reporters, mutations were identified in cgs1 in a genetic screen for suppressors of an adenylate cyclase deletion allele¹⁹, and mutations in cgs2 in a genetic screen for suppressors of an activation-defective form of adenylate cyclase¹⁰. Thus, a system involving transcriptional regulation of fbp1 is capable of identifying mutations that either reduce or increase PKA activity in the cell.

### Recombinant Fission Yeast Containing Reporter Constructs

Translational fusions carrying the fbp1 promoter fused to the S. pombe ura4 and the E. coli lacZ reporter genes can be used to monitor the yeast cell's ability to detect glucose. Additional reporter genes can be used in methods and cells of the invention, including, but not limited to: genes that encode fluorescent proteins and other biosynthetic pathway genes such as his3²⁰. These constructs can be integrated in single copy into the S. pombe genome, creating stable reporters of fbp1 transcription¹⁷. The ura4 gene encodes OMP decarboxylase, which is required for uracil biosynthesis and for sensitivity to the pyrimidine analog 5-fluoro-orotic acid (5FOA). Thus, the fbp1-ura4 fusion acts as a selectable or counterselectable marker, making it extremely useful in genetic screens for mutations or clones that increase or decrease fbp1 transcription. The fbp1-ura4 fusion, for example, can be inserted in single copy into the S. pombe genome at the fbp1 locus and disrupting the wild type fbp1 gene. The lacZ gene encodes β-galactosidase, allowing sensitive and rapid assays of expression from the fbp1 promoter that are consistent with direct examination of fbp1⁺ mRNA levels. The fbp1-lacZ fusion, for example, can be inserted in single copy into the S. pombe genome at the ura4 locus so as to disrupt the wild type ura4 gene, such that all Ura4 enzyme activity in these cells comes from the fbp1-ura4 fusion.

Strains carrying these fusions can be easily assessed for their ability to regulate fbp1 transcription. Strains that glucose-repress fbp1-ura4 transcription cannot form single colonies on a glucose-rich medium lacking uracil because high glucose inhibits OMP decarboxylase expression, thereby reducing uracil biosynthesis. The same strains grow on a glucose-rich medium containing 5FOA because ura4 expression is required for 5FOA sensitivity. Strains that fail to glucose-repress fbp1-ura4 form Ura⁺ colonies on a glucose-rich medium lacking uracil.

In some embodiments, the recombinant fission yeast cell has only a single reporter construct, such as the fbp1-ura4 fusion construct, which can be employed to detect alterations of cAMP level in the cell, and thus inhibition or activation of PDE. Glucose repression of fbp1 is cAMP dependent. High glucose concentrations stimulate adenylate cyclase activity and therefore raise cAMP levels, which stimulate cAMP-dependent protein kinase (PKA) activity. Elevated PKA activity in turn leads to fbp1 repression. Therefore, with the appropriate genetic background providing the appropriate cAMP levels, the growth phenotype of a recombinant fission yeast cell containing the fbp1-ura4 fusion construct can be used to monitor changes in PDE activity. Inhibiting PDE activity will raise cAMP levels, and in a cell possessing the fbp1-ura4 construct inhibiting PDE activity will result in greater glucose-induced repression of Ura4 activity. One consequence of reduced Ura4 activity is loss of 5FOA sensitivity. Thus, in one embodiment, a recombinant fission yeast cell containing a fbp1-ura4 fusion construct is used to identify chemical inhibitors of PDE. When grown in the presence of a test compound which is an inhibitor of PDE, the yeast cell loses 5FOA sensitivity, and therefore grows in the presence of 5FOA when treated with the test compound, but does not grow in 5FOA containing medium in the absence of the test compound.

In other embodiments, the fission yeast cell also has incorporated into its genome a second construct, such as the fbp1-lacZ fusion construct. If the fbp1 promoter is used for both constructs, this permits quantitative monitoring of fbp1+ expression through measurement of β-galactosidase activity. Thus, in one embodiment, a recombinant fission yeast cell contains both an fbp1-ura4 fusion construct and an fbp1-lacZ fusion construct. The level of inhibition of PDE by a test compound can be monitored quantitatively by measuring β-galactosidase activity in the presence of the test compound. The greater the inhibition of PDE, the higher will be the cAMP level in the cell, and consequently, due to cAMP-dependent repression of the fbp1-lacZ construct, the lower will be the β-galactosidase activity. In one embodiment, cells are preincubated, e.g., overnight, in medium containing 1-5 mM cAMP to repress transcription of an fbp1-lacZ reporter construct from the fbp1 promoter and consequently repress β-galactosidase activity. Cyclic AMP then can be washed out by tranferring the cells to medium without cAMP at time 0. Washout of cAMP stimulates expression of β-galactosidase to an extent depending on the cellular machinery controlling cAMP levels, including PDE activity.

Alternatively, in a cell possessing both the fbp1-ura4 and fbp1-lacZ constructs, the fbp1 promoter can be used for the ura4 fusion, while a constitutive promoter (e.g., the his7 promoter) can be used to drive a fluorescent protein fusion. In this way, fluorescence can be used to quantitate cell growth. Thus, in another embodiment, a recombinant fission yeast cell contains an fbp1-ura4 fusion construct driven by an fbp1 promoter and an fbp1-lacZ fusion construct driven by a constitutive promoter. The cell can be used to identify an inhibitor of PDE and to quantitate the degree of inhibition. The growth phenotype of the cell can be used to identify test compounds that inhibit PDE; for example, when grown in the presence of a test compound that inhibits PDE, the growth phenotype can switch from 5FOA sensitive to 5FOA tolerant. The amount of growth can be quantified using the fluorescence emission of a fluorescent reporter protein. For example, the greater the amount of fluorescence when grown in the presence of 5FOA, the greater the extent of PDE inhibition by the test compound.

Mutations That Modify cAMP Levels in Fission Yeast

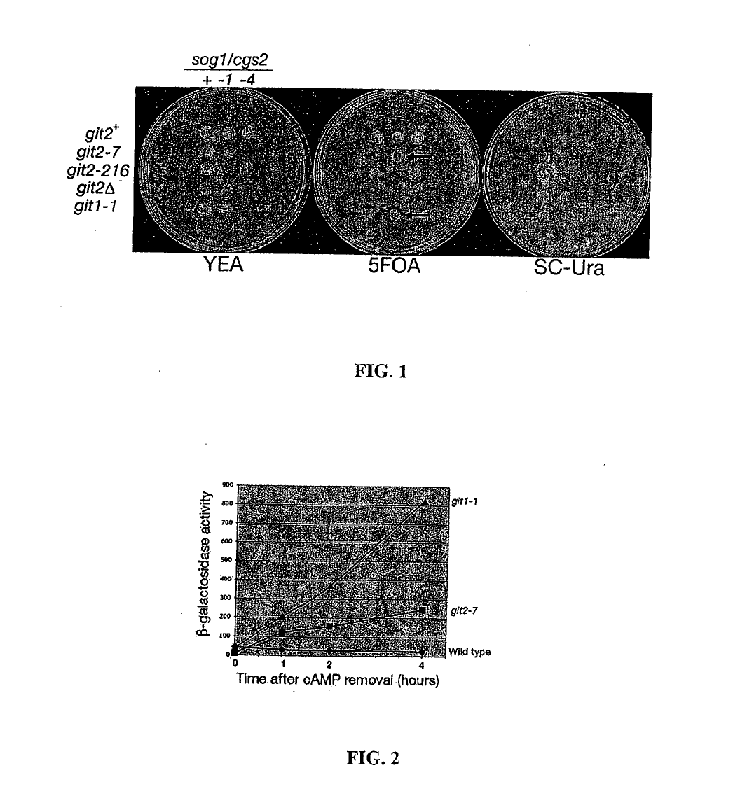

In general, mutations have been identified in nine git genes (git=glucose insensitive transcription) required for glucose repression in fission yeast¹⁷. The increase in fbp1-ura4 expression in git⁻ strains confers a 5FOA-sensitive phenotype that is suppressed by clones carrying the wild type copy of the defective git gene in the host strain or a multicopy suppressor^{13, 14, 16, 19, 21-23}. The gene git2 (cyr1) encodes adenylate cyclase, and git6 (pka1) encodes the catalytic subunit of protein kinase A (PKA). Moreover, git1, git3, git5, git7, git8 git10, and git11 are all required for adenylate cyclase activation. Some “upstream” git genes encode a GPCR (git3) and its cognate G protein composed of the Gpa2 Gα, the Git5 Gβ, and the Git11 Gγ. The role of these four genes is to activate the Gpa2 Gα, as mutational activation of Gpa2 suppresses deletions of the other three genes. Since Git1, Git7, and Git10 are still required for glucose repression in a strain expressing an “activated” Gpa2, these proteins may act independently of the G protein or are required for Gpa2 activation of adenylate cyclase. In general, strains that are Ura⁺ and 5FOA-sensitive have reduced cAMP levels (either basal or glucose-stimulated) as compared with wild type strains (see also Table 1, FIG. 1, and Example 1).

While strains that have increased PKA activity are defective in fbp1-ura4 transcription, they largely resemble wild type strains, as it is only under glucose-starvation conditions that a defect in fbp1 transcription is evident. However, by starting with strains with reduced cAMP levels and thus elevated fbp1 expression, mutations have been identified in genes that reduce fbp1-ura4 expression, conferring 5FOA-resistant growth upon the originally 5FOA-sensitive mutant strain. The cgs1⁺ gene, encoding the PKA regulatory subunit, was identified in a screen for suppressors of an adenylate cyclase deletion^{18, 19}. Strains carrying cgs1 mutations fail to express fbp1 even when cAMP levels are high. The cgs2⁺ gene, encoding the only PDE gene in S. pombe, was identified in a screen for suppressors of a catalytically active form of adenylate cyclase that fails to be stimulated by glucose^{19, 24}. Three different mutant alleles of cgs2⁺ have been identified. These mutations reduce PDE activity to different levels and lead to an increase in cAMP levels that is dependent upon the function of adenylate cyclase (Table 1, FIG. 1). A genetic screen has been carried out for activated alleles of the gpa2 Gα gene that bypass the requirement for the Gβγ dimer or Git3 GPCR. These alleles, along with the gpa2^{R176H} GTPase deficient allele, elevate cAMP signaling by raising cAMP levels in the cells (Table 1).

In some embodiments, the recombinant fission yeast cell is a pap1Δcell. In a pap1Δ cell, the pap1⁺ gene has been deleted. The deletion of the pap1⁺ gene is not essential for high throughput screening, however it may make the cells more sensitive to both 5FOA and to drug treatment. This pap1⁺ gene encodes a transcriptional activator that regulates the expression of ABC transporter genes. Loss of this gene may allow compounds to accumulate in S. pombe. In certain embodiments, a cell of the invention is a pap1⁺ cell, and therefore does not have the pap1⁺ gene deletion.

### Introduction of Exogenous PDE Genes

Recombinant strains of fission yeast can be prepared in which the S. pombe PDE gene is replaced with an exogenous PDE gene to be used for screening to identify chemical or biological modulators of an exogenous PDE activity. Standard yeast manipulations of the genomic DNA, which are well known in the art, can be employed to replace the cgs2⁺ gene with that of an exogenous, e.g., a mammalian or protozoan, PDE gene (or to knock out the cgs2⁺ gene and introduce an exogenous PDE at another site). Typically, this is done in two steps. First, a construct expressing both a selectable marker and a counterselectable marker is introduced by homologous recombination at the cgs2⁺ site, and cells are selected for expression of the marker. These cells will have lost Cgs2 expression and therefore have lost endogenous PDE activity. Second, the exogenous PDE gene is exchanged for the construct added in the first step. The counterselectable marker then can be used to isolate cells having the exogenous PDE gene. As an alternative to replacing the marker at the cgs2 genetic locus with the exogenous PDE gene, the exogenous PDE gene can be integrated into a second genetic locus of a cgs2″ mutant strain.

In some embodiments, the ura4⁺ gene serves as both the selectable marker and the counterselectable marker. The cgs2⁺ gene is replaced with the ura4⁺ gene by homologous recombination. Cells having incorporated ura4⁺ are selected based on their growth in the absence of uracil. Next, the exogenous PDE gene is amplified by PCR using oligonucleotides that possess homology to the cgs2 locus, and the exogenous PDE replaces ura4⁺ by homologous recombination. Cells in which the PDE gene has replaced the ura4⁺ gene at cgs2 can be selected on 5FOA-containing plates (i.e., cells incorporating the PDE gene are 5FOA-insensitive, but cells retaining ura4⁺ are 5FOA-sensitive). In another embodiment, the selectable marker is the his7⁺ gene and the counterselectable marker is TK (thymidine kinase). In that case, cells containing his7⁺ can be selected based on growth in the absence of histidine, and TK can be counterselected based on TK-induced sensitivity to 5-fluoro-2-dexoyuridine (FUdr)²⁵.

After the cgs2⁺ gene has been inactivated, and an exogenous PDE gene has been introduced, the resulting yeast cell can be crossed with a yeast cell that contains a reporter construct by standard genetic crosses. The reporter construct encodes a reporter gene whose expression reflects cAMP levels in the cell. For example the reporter construct can be an fbp1-ura4 fusion reporter construct. A second reporter construct, e.g., an fbp1-lacZ fusion construct, can also be added by crossing. For these crosses, a fission yeast background strain can be selected which has a sufficiently high level of adenylate cyclase activation such that the exogenous PDE activity can support a 5FOA-sensitive growth behavior. For example, if the exogenous PDE activity is similar to that of the normal yeast PDE, even a weak mutation, such as the loss of git11 (see Table 1), would confer 5FOA-sensitive growth. If, however, the exogenous PDE activity is relatively low, a greater defect in the cAMP pathway, such as the loss of the git3 or gpa2 genes (Table 1), could be required to confer 5FOA-sensitive growth. Should the PDE be so weak that even loss of the gpa2 gene does not confer 5FOA-sensitivity, a deletion of the adenylate cyclase gene could be incorporated and endogenous cAMP production could be replaced by exogenous cAMP addition to create the conditions needed for a PDE inhibitor screen. If the PDE is very active, it may confer 5FOA-sensitivity even in a wild type background. In this case, activated forms of the gpa2 gene (Welton and Hoffman, supra) can be introduced to increase cAMP production, in order to make the cells more sensitive to changes in the PDE activity.

### Screening Assays

The recombinant fission yeast cells described above can be used in high throughput chemical screens to identify PDE inhibitors that confer 5FOA-resistant growth.

Screening assays can be adapted from the use of solid media to working in liquid media in microtiter plates suitable for chemical library screening. PDE inhibitors would confer increased optical density in the affected wells due to cell growth, along with increased fluorescence from a constitutively expressed fluorescent protein reporter. Preferably, a positive growth screen is used, such as growth in the absence of uracil or in the presence of 5FOA, so that compounds that are toxic to the cells or impermeable will not yield a positive result and can be avoided.

Test compounds or agents to be screened can be naturally occurring or synthetic molecules. The activity of the compounds can be known or unknown. Test compounds can be obtained from natural sources, such as, for example, marine microorganisms, algae, plants, fungi, etc. Test compounds can include, for example, pharmaceuticals, therapeutics, environmental, agricultural, or industrial agents, pollutants, cosmeceuticals, drugs, organic compounds, lipids, fatty acids, steroids, glucocorticoids, antibiotics, peptides, proteins, sugars, carbohydrates, chimeric molecules, purines, pyrimidines, derivatives, structural analogs, or combinations thereof.

Collections of compounds known as libraries can be used for screening. Libraries of natural compounds in the form of bacterial, fungal, plant and animal extracts are available from governmental or private sources or can be produced readily. Alternatively, agents to be assayed can be from combinatorial libraries of agents, including peptides or small molecules, or from existing repertories of chemical compounds synthesized in industry, e.g., by the chemical, pharmaceutical, environmental, agricultural, marine, drug, and biotechnological industries. Preparation of combinatorial chemical libraries is well known to those of skill in the art. Compounds that can be synthesized for combinatorial libraries include polypeptides, proteins, nucleic acids, beta-turn mimetics, polysaccharides, phospholipids, hormones, prostaglandins, steroids, aromatic compounds, heterocyclic compounds, benzodiazepines, oligomeric N-substituted glycines, and oligocarbamates. Devices for the preparation of combinatorial libraries are commercially available (see, e.g., 357 MPS, 390 MPS, Advanced Chem Tech, Louisville, Ky, Symphony, Rainin, Woburn, Mass., 433A Applied Biosystems, Foster City, Calif., 9050 Plus, Millipore, Bedford, Mass.). Additionally, natural or synthetically produced libraries and compounds are readily modified through conventional chemical, physical and biochemical means, and may be used to produce combinatorial libraries.

Screening may also be directed to known pharmacologically active compounds and analogs thereof. Known pharmacological agents may be subjected to directed or random chemical modifications, such as acylation, coalkylation, esterification, amidification, etc. to produce structural analogs. New potential test agents may also be created using methods such as rational drug design or computer modeling.

As described above, compounds that may be assayed according to the methods of the presently disclosed embodiments encompass numerous chemical classes. For example, organic molecules, preferably small organic compounds having a molecular weight less than about 2,500 daltons, are a type of compound for use in the methods of the presently disclosed embodiments.

In the methods of the presently disclosed embodiments, each test compound, or a composition comprising the test compound, is brought into contact with a cell or plurality of cells in a manner such that the test compound is capable of exerting activity on at least a substantial portion of, if not all of, the individual cells. By substantial portion is meant at least 75%, usually at least 80%, and in many embodiments 90% or 95% or higher percentage of the cells are exposed to the test compound. Generally, a cell is contacted with a test compound in a manner such that the compound is internalized by the cells. For example, the test compound can be added into a growth medium or incubation solution in which the cell is suspended or upon which the cell is growing. Compounds are generally screened at a concentration in the range expected for them to be effective, e.g., as PDE inhibitors, or somewhat above that concentration. Any concentration below 1 mM may be chosen, but screening assays are often conducted with test compounds at about 7 μM, about 20 μM, or about 50 μM.

In order to screen for biological modulators of an exogenous PDE, cDNA libraries can be constructed in a fission yeast plasmid expression vector such as pLEV3²⁶. These libraries would include cDNA from specific tissues encoding candidate modulators of PDE activity. Such modulators can be the targets of subsequent drug screens and may represent novel drug targets. This class of drug target may be expressed in a subset of cell types or tissues while the PDE may be expressed in a wider range of cell types. As such, targeting the modulator may limit the effect on PDE activity to that expressed in the desired tissue, thus reducing side effects relative to drugs that directly target the PDE in cells in which it is expressed.

Techniques for producing and probing nucleic acid sequence libraries are described, for example, in Sambrook et al., Molecular Cloning: A Laboratory Manual, 2nd Ed., (1989) Cold Spring Harbor Press, Cold Spring Harbor, N.Y. The cDNA library can be made from poly-adenylated mRNA by using poly-T primers to prepare cDNA from the mRNA. Libraries of cDNA are made from fission yeast or from selected tissues. Many cDNA libraries are available commercially. The choice of cell type for library construction can be made, for example, based on the location of a target PDE whose inhibition might be useful to treat a particular disease. Libraries of genomic DNA also can be utilized. Genomic libraries can be used in vectors suitable for carrying large segments of a genome, such as P1 or YAC, as described in detail in Sambrook et al., 9.4-9.30. Either cDNA or genomic libraries can be inserted into a suitable expression vector and used to transform fission yeast. Such transformed yeast can be screened using the methods of the presently disclosed embodiments, in order to identify biological activators or biological inhibitors of PDE. For identifying biological activators or inhibitors of a mammalian exogenous PDE, cDNA libraries obtained from human or another mammal are preferred.

After high throughput screening (primary screening), several candidate inhibitors or activators of PDE will have been identified. These inhibitor or activator compounds can be further tested using a secondary screen, such as an in vitro assay wherein the compounds are tested using purified PDE under controlled conditions. The secondary screen can further identify the most desirable compounds, for example those with the highest potency (e.g., lowest K₁ value for an inhibitor compound).

### PDE-Modulating Compounds

Methods of the invention involve the administration of compounds that modulate the activity of PDEs. In certain embodiments the hydrolysis of the substrate 3′,5′-cyclic adenosine monophosphate (cAMP) to yield 5′-adenosine monophosphate or the hydrolysis of another substrate, such as 3′,5′-cyclic guanosine monophosphate (cGMP) is modulated. Compositions of the invention include compounds that modulate or inhibit PDE activity in vitro or in vivo, in cells, tissues, or subjects, which may be mammals or humans. As used herein, the term “PDE-inhibiting compounds” means compounds that reduce PDE hydrolysis of its substrate, which in some embodiments may be cAMP and in certain embodiments may be cGMP. The methods of the invention, in some aspects, involve the administration of a PDE-inhibiting compound and are useful to reduce or prevent adverse effects that are associated with abnormal levels of PDE substrates such as cAMP and/or cGMP, for example, cell death and/or damage or disease.

As used herein, the term “PDE-associated disease or disorder” includes, but is not limited to diseases and disorders in which there is abnormal PDE activity and/or abnormal levels of a substrate of a PDE, such as cAMP and/or cGMP. As used herein, the term “PDE activity” means PDE-mediated hydrolysis of a substrate such as cAMP or cGMP. An abnormal level of PDE activity and/or an abnormal level of a substrate may be a level that is higher than a normal level or may be a level that is lower than a normal level, wherein a “normal” level is the level in a subject who does not have a disease or disorder associated with PDE activity or with an abnormal level of cAMP or cGMP. Disease or disorders that may be associated with PDE activity and abnormal cAMP or cGMP levels, and which may benefit from treatment according to the methods described herein using compounds of the invention, are: transplant rejection (such as organ transplant, acute transplant, xenotransplant or heterograft or homograft such as is employed in burn treatment); protection from ischemic or reperfusion injury such as ischemic or reperfusion injury incurred during organ transplantation, myocardial infarction, stroke or other causes; transplantation tolerance induction; arthritis (such as rheumatoid arthritis, psoriatic arthritis or osteoarthritis); multiple sclerosis; respiratory and pulmonary diseases including but not limited to asthma, exercise induced asthma, chronic obstructive pulmonary disease (COPD), emphysema, bronchitis, and acute respiratory distress syndrome (ARDS); inflammatory bowel disease, including ulcerative colitis and Crohn's disease; lupus (systemic lupus erythematosis); graft vs. host disease; T-cell mediated hypersensitivity diseases, including contact hypersensitivity, delayed-type hypersensitivity, and gluten-sensitive enteropathy (Celiac disease); psoriasis; contact dermatitis (including that due to poison ivy); Hashimoto's thyroiditis; Sjogren's syndrome; Autoimmune Hyperthyroidism, such as Graves' Disease; Addison's disease (autoimmune disease of the adrenal glands); Autoimmune polyglandular disease (also known as autoimmune polyglandular syndrome); autoimmune alopecia; pernicious anemia; vitiligo; autoimmune hypopituatarism; Guillain-Barre syndrome; other autoimmune diseases; glomerulonephritis; serum sickness; uticaria; allergic diseases such as respiratory allergies (e.g., asthma, hayfever, allergic rhinitis) or skin allergies; scieracierma; mycosis fungoides; acute inflammatory and respiratory responses (such as acute respiratory distress syndrome and ishchemia/reperfusion injury); dermatomyositis; alopecia greata; chronic actinic dermatitis; eczema; Behcet's disease; Pustulosis palmoplanteris; Pyoderma gangrenum; Sezary's syndrome; atopic dermatitis; systemic schlerosis; and morphea, and cancer, but are not so limited.

Other examples of diseases and disorders associated with cAMP PDE activity and/or abnormal cAMP or cGMP levels include, but are not limited to neurodegenerative disorders, penile erectile dysfunction, anxiety, depression, Alzheimer's disease, Parkinson's disease, Huntington's disease, schizophrenia, psychosis, sepsis, renal disease, memory loss, chronic lymphocytic leukemia, prostate cancer, thyroid disease, male hypogonadism, cardiac disease, diabetes, obesity, osteoporosis, and cystic fibrosis.

Deleterious effects seen in these diseases and/or disorders that are triggered by abnormal PDE activity and/or abnormal levels of a substrate of a PDE (e.g., cAMP or cGMP) may be ameliorated by the administration of compounds and/or compositions that modulate PDE activity. The compounds or compositions may comprise for example at least one PDE inhibitor, which may be selective for a PDE family, a specific PDE subfamily, or a specific isoform of a PDE-subfamily member, such as a selective PDE4 inhibitor or a selective PDE7 inhibitor, or a dual PDE7-PDE4 inhibitor.

Compounds of the invention include compounds that modulate PDE activity in the hydrolysis of substrates such as cAMP and cGMP in cells and/or tissues (in a subject), thereby reducing the cell and/or tissue damage and/or clinical manifestations of a PDE-associated disease or disorder. In some embodiments of the invention, the compounds inhibit PDE activity, thus resulting in an increase in levels of cAMP and/or cGMP.

A compound of the invention may be an isolated compound. By “isolated”, it is meant present in sufficient quantity to permit its identification or use according to the procedures described herein. Because an isolated material may be admixed with a carrier in a preparation, such as, for example, for adding to a sample or for analysis, the isolated material may comprise only a small percentage by weight of the preparation.

In some aspects of the invention, one or more of compounds described herein may be administered to a subject that is free of indications for a previously determined use of the compounds. By “free of indications for a previously determined use”, it is meant that the subject does not have symptoms that call for treatment with one or more of the compounds of the invention for a previously determined use of that compound, other than the indication that exists as a result of this invention. As used herein the term “previously determined use” of a compound means the use of the compound that was previously identified. Thus, the previously determined use is not the use of inhibiting PDE activity and/or increasing the level of a PDE substrate such as cAMP and/or cGMP.

### Administration and Delivery of PDE Modulating Compounds

Methods of the invention, in some aspects, include administration of a PDE-inhibiting compound that preferentially targets neuronal or vascular cells and/or tissues or other specific cell or tissue types. In addition, the compounds can be specifically targeted to neuronal or vascular tissue or other specific tissue types. The targeting may be done using various delivery methods, including, but not limited to: administration to neuronal or vascular tissue or other specific target tissue, the addition of targeting molecules to direct the compounds of the invention to neuronal or other tissues (e.g. glial cells, nerve cells, vascular cells, etc.). Additional methods to specifically target compounds and compositions of the invention to specific tissues, such as neuronal tissues, vascular tissues, or other types of tissues may also be used with the compounds and compositions of the invention, and are known to those of ordinary skill in the art.

In certain embodiments the invention provides compounds that inhibit PDE activity in cells, tissues, and/or subjects and the use of such compounds to inhibit PDE. PDE inhibitors of the invention, such as selective PDE4 inhibitors or selective PDE7 inhibitors, or a dual PDE7-PDE4 inhibitors, may be used for treatment of cells, tissues, and/or subjects and for research purposes. As used herein, the term “PDE activity” means the hydrolysis of PDE substrate such as cAMP and/or cGMP. It is understood that increased activity of a PDE may result in an abnormally low level of cAMP or cGMP. Also, it will be understood, that for reasons unrelated to the activity of a PDE in a cell, tissue or subject, a level of cAMP and/or cGMP may be below a desirable level (e.g., at an abnormally low level) and methods and compounds of the invention may be used to inhibit PDE activity and thereby increase the level of cAMP and/or cGMP in the cell, tissue, or subject.

PDE-inhibiting compounds of the invention may be administered to a subject to reduce the risk of a PDE-associated disorder. Reducing the risk of a disorder associated with above-normal PDE activity or a associated with abnormally low levels of a substrate of a PDE (e.g., cAMP and/or cGMP), means using treatments and/or medications that include compounds of the invention, such as compounds comprising selective PDE4 inhibitors or selective PDE7 inhibitors, or a dual PDE7-PDE4 inhibitors, to reduce PDE activity levels, therein increasing the subject's levels of the substrate, e.g., cAMP and/or cGMP and thus treating the associated disease or disorder.

As used herein, the term “subject” means any mammal that may be in need of treatment with a PDE-modulating or inhibiting compound of the invention. Subjects include but are not limited to: humans, non-human primates, cats, dogs, sheep, pigs, horses, cows, rodents such as mice, and rats.

As used herein the term “inhibit” means to reduce the amount of PDE activity to a level or amount that is statistically significantly less than an initial level, which may be a control level of PDE activity and/or PDE substrate hydrolysis. As used herein, an initial level may be a level in a cell, tissue, or subject not contacted with a PDE-inhibiting compound of the invention. In some cases, the decrease in the level of PDE activity and/or PDE substrate hydrolysis means the level of PDE activity and/or substrate hydrolysis is reduced from an initial level to a level significantly lower than the initial level. In some embodiments, the reduced level may be zero.

A PDE-modulating compound of the invention (e.g., a PDE inhibitor, such as a selective PDE4 inhibitor or a selective PDE7 inhibitor, or a dual PDE7-PDE4 inhibitor) may be used to treat a subject with a PDE-associated disease or disorder. As used herein, the term “treat” includes active treatment of a subject that has a PDE-associated disease or disorder (e.g., a subject diagnosed with such a condition) and also includes prophylactic treatment of a subject who is has not yet been diagnosed and/or has not yet developed a PDE-associated disease. Compounds of the invention may administered prophylactically to a subject at risk of a PDE-associated disease or disorder. Determination of a subject at risk for a PDE-associated disease or disorder, and/or the determination of a diagnosis of a PDE-associated disease or disorder in a subject, may be carried out by one of ordinary skill in the art using routine methods.

A PDE-modulating or inhibiting compound of the invention may be delivered to a cell using standard methods known to those of ordinary skill in the art. Various techniques may be employed for introducing PDE-modulating compounds of the invention to cells, depending on whether the compounds are introduced in vitro or in vivo in a host.

When administered, the PDE-modulating compounds (also referred to herein as therapeutic compounds and/or pharmaceutical compounds) of the present invention are administered in pharmaceutically acceptable preparations. Such preparations may routinely contain pharmaceutically acceptable concentrations of salt, buffering agents, preservatives, compatible carriers, and optionally other therapeutic agents.

The term “pharmaceutically acceptable” carrier means a non-toxic material that does not interfere with the effectiveness of the biological activity of the active ingredients. The characteristics of the carrier will depend on the route of administration.

The therapeutics of the invention can be administered by any conventional route, including injection or by gradual infusion over time. The administration may for example, be oral, intravenous, intraperitoneal, intrathecal, intramuscular, intranasal, intracavity, subcutaneous, intradermal, mucosal, transdermal, or transdermal.

The therapeutic compositions may conveniently be presented in unit dosage form and may be prepared by any of the methods well known in the art of pharmacy. All methods include the step of bringing the compounds into association with a carrier which constitutes one or more accessory ingredients. In general, the compositions are prepared by uniformly and intimately bringing the therapeutic agent into association with a liquid carrier, a finely divided solid carrier, or both, and then, if necessary, shaping the product.

Compositions suitable for parenteral administration conveniently comprise a sterile aqueous preparation of the therapeutic agent, which is preferably isotonic with the blood of the recipient. This aqueous preparation may be formulated according to known methods using those suitable dispersing or wetting agents and suspending agents. The sterile injectable preparation may also be a sterile injectable solution or suspension in a non-toxic parenterally-acceptable diluent or solvent, for example as a solution in 1,3-butane diol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono or di-glycerides. In addition, fatty acids such as oleic acid find use in the preparation of injectables. Carrier formulations suitable for oral, subcutaneous, intravenous, intramuscular, etc. can be found in Remington's Pharmaceutical Sciences, Mack Publishing Company, Easton, Pa.

Compositions suitable for oral administration may be presented as discrete units such as capsules, cachets, tablets, or lozenges, each containing a predetermined amount of the therapeutic agent. Other compositions include suspensions in aqueous liquors or non-aqueous liquids such as a syrup, an elixir, or an emulsion.

In some embodiments of the invention, a PDE-modulating compound of the invention may be delivered in the form of a delivery complex. The delivery complex may deliver the PDE-modulating compound into any cell type, or may be associated with a molecule for targeting a specific cell type. Examples of delivery complexes include a PDE-modulating compound of the invention associated with: a sterol (e.g., cholesterol), a lipid (e.g., a cationic lipid, virosome or liposome), or a target cell specific binding agent (e.g., an antibody, including but not limited to monoclonal antibodies, or a ligand recognized by target cell specific receptor). Some complexes may be sufficiently stable in vivo to prevent significant uncoupling prior to internalization by the target cell. However, the complex can be cleavable under appropriate conditions within the cell so that the PDE-modulating compound is released in a functional form.

An example of a targeting method, although not intended to be limiting, is the use of liposomes to deliver a PDE-modulating compound of the invention into a cell. Liposomes may be targeted to a particular tissue, such neuronal cells, (e.g. hippocampal cells, etc), or other cell type, by coupling the liposome to a specific ligand such as a monoclonal antibody, sugar, glycolipid, or protein. Such proteins include proteins or fragments thereof specific for a particular cell type, antibodies for proteins that undergo internalization in cycling, proteins that target intracellular localization and enhance intracellular half life, and the like.

For certain uses, it may be desirable to target the compound to particular cells, for example specific neuronal cells, including specific tissue cell types, e.g. tissue-specific nervous system cells. In some embodiments, it may be desirable to target a PDE-modulating compound to another cell type, including, but not limited to, cardiac cells, pancreatic cells, vascular cells, etc. In such instances, a vehicle (e.g. a liposome) used for delivering a PDE-modulating compound of the invention to a cell type (e.g. a neuronal cell, vascular cell, etc.) may have a targeting molecule attached thereto that is an antibody specific for a surface membrane polypeptide of the cell type or may have attached thereto a ligand for a receptor on the cell type. Such a targeting molecule can be bound to or incorporated within the PDE-modulating compound delivery vehicle. Where liposomes are employed to deliver a PDE-modulating compound of the invention, proteins that bind to a surface membrane protein associated with endocytosis may be incorporated into the liposome formulation for targeting and/or to facilitate uptake.

Liposomes are commercially available from Invitrogen, for example, as LIPOFECTIN™ and LIPOFECTACE™, which are formed of cationic lipids such as N-[1-(2,3 dioleyloxy)-propyl]-N,N, N-trimethylammonium chloride (DOTMA) and dimethyl dioctadecylammonium bromide (DDAB). Methods for making liposomes are well known in the art and have been described in many publications.

The invention provides a composition of the above-described agents for use as a medicament, methods for preparing the medicament and methods for the sustained release of the medicament in vivo. Delivery systems can include time-release, delayed release or sustained release delivery systems. Such systems can avoid repeated administrations of the therapeutic agent of the invention, increasing convenience to the subject and the physician. Many types of release delivery systems are available and known to those of ordinary skill in the art. They include, but are not limited to, polymer-based systems such as polylactic and polyglycolic acid, poly(lactide-glycolide), copolyoxalates, polyanhydrides, polyesteramides, polyorthoesters, polyhydroxybutyric acid, and polycaprolactone. Microcapsules of the foregoing polymers containing drugs are described in, for example, U.S. Pat. No. 5,075,109. Nonpolymer systems that are lipids including sterols such as cholesterol, cholesterol esters and fatty acids or neutral fats such as mono-, di- and tri-glycerides; phospholipids; hydrogel release systems; silastic systems; peptide based systems; wax coatings, compressed tablets using conventional binders and excipients, partially fused implants and the like. Specific examples include, but are not limited to: (a) erosional systems in which the polysaccharide is contained in a form within a matrix, found in U.S. Pat. Nos. 4,452,775, 4,675,189, and 5,736,152, and (b) diffusional systems in which an active component permeates at a controlled rate from a polymer such as described in U.S. Pat. Nos. 3,854,480, 5,133,974 and 5,407,686. In addition, pump-based hardware delivery systems can be used, some of which are adapted for implantation.

In one particular embodiment, the preferred vehicle is a biocompatible microparticle or implant that is suitable for implantation into the mammalian recipient. Exemplary bioerodible implants that are useful in accordance with this method are described in PCT International application no. WO 95/24929, entitled “Polymeric Gene Delivery System”. describes a biocompatible, preferably biodegradable polymeric matrix for containing an exogenous gene under the control of an appropriate promoter. The polymeric matrix is used to achieve sustained release of the exogenous gene in the patient. In accordance with the instant invention, the compound(s) of the invention is encapsulated or dispersed within the biocompatible, preferably biodegradable polymeric matrix disclosed in WO 95/24929. The polymeric matrix preferably is in the form of a microparticle such as a microsphere (wherein the compound is dispersed throughout a solid polymeric matrix) or a microcapsule (wherein the compound is stored in the core of a polymeric shell). Other forms of the polymeric matrix for containing the compounds of the invention include films, coatings, gels, implants, and stents. The size and composition of the polymeric matrix device is selected to result in favorable release kinetics in the tissue into which the matrix device is implanted. The size of the polymeric matrix device further is selected according to the method of delivery which is to be used. The polymeric matrix composition can be selected to have both favorable degradation rates and also to be formed of a material which is bioadhesive, to further increase the effectiveness of transfer when the device is administered to a vascular surface. The matrix composition also can be selected not to degrade, but rather, to release by diffusion over an extended period of time.

Both non-biodegradable and biodegradable polymeric matrices can be used to deliver agents and compounds of the invention of the invention to the subject. Biodegradable matrices are preferred. Such polymers may be natural or synthetic polymers. Synthetic polymers are preferred. The polymer is selected based on the period of time over which release is desired, generally in the order of a few hours to a year or longer. Typically, release over a period ranging from between a few hours and three to twelve months is most desirable. The polymer optionally is in the form of a hydrogel that can absorb up to about 90% of its weight in water and further, optionally is cross-linked with multi-valent ions or other polymers.

In general, the agents and/or compounds of the invention are delivered using the bioerodible implant by way of diffusion, or more preferably, by degradation of the polymeric matrix. Exemplary synthetic polymers which can be used to form the biodegradable delivery system include: polyamides, polycarbonates, polyalkylenes, polyalkylene glycols, polyalkylene oxides, polyalkylene terepthalates, polyvinyl alcohols, polyvinyl ethers, polyvinyl esters, polyvinyl halides, polyvinylpyrrolidone, polyglycolides, polysiloxanes, polyurethanes and co-polymers thereof, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulphate sodium salt, poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate), polyethylene, polypropylene, poly(ethylene glycol), poly(ethylene oxide), poly(ethylene terephthalate), poly(vinyl alcohols), polyvinyl acetate, poly vinyl chloride, polystyrene and polyvinylpyrrolidone.

Examples of non-biodegradable polymers include ethylene vinyl acetate, poly(meth)acrylic acid, polyamides, copolymers and mixtures thereof.

Examples of biodegradable polymers include synthetic polymers such as polymers of lactic acid and glycolic acid, polyanhydrides, poly(ortho)esters, polyurethanes, poly(butic acid), poly(valeric acid), and poly(lactide-cocaprolactone), and natural polymers such as alginate and other polysaccharides including dextran and cellulose, collagen, chemical derivatives thereof (substitutions, additions of chemical groups, for example, alkyl, alkylene, hydroxylations, oxidations, and other modifications routinely made by those skilled in the art), albumin and other hydrophilic proteins, zein and other prolamines and hydrophobic proteins, copolymers and mixtures thereof. In general, these materials degrade either by enzymatic hydrolysis or exposure to water in vivo, by surface or bulk erosion.

Bioadhesive polymers of particular interest include bioerodible hydrogels may include, but are not limited to: polyhyaluronic acids, casein, gelatin, glutin, polyanhydrides, polyacrylic acid, alginate, chitosan, poly(methyl methacrylates), poly(ethyl methacrylates), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate).

Use of a long-term sustained release implant may be particularly suitable for treatment of subjects with an established neurological disorder or other cAMP PDE-associated disease or disorder as well as subjects at risk of developing a such a disease or disorder.

“Long-term” release, as used herein, means that the implant is constructed and arranged to deliver therapeutic levels of the active ingredient for at least 7 days, and preferably 30-60 days, and most preferably months or years. The implant may be positioned at or near the site of the neurological damage or the area of the brain or nervous system affected by or involved in the neurodegenerative disorder. Long-term release implants may also be used in non-neuronal tissues and organs to allow regional administration of a PDE-modulating compound of the invention. Long-term sustained release implants are well known to those of ordinary skill in the art and include some of the release systems described above.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

PDE inhibitor compounds described herein, include salts, prodrugs and solvates. The term “salt(s)”, as employed herein, denotes acidic and/or basic salts formed with inorganic and/or organic acids and bases and Zwitterions (internal or inner salts) are also included. Also included herein are quaternary ammonium salts such as alkylammonium salts. Pharmaceutically acceptable (i.e., non-toxic, physiologically acceptable) salts are preferred.

Exemplary acid addition salts include acetates (such as those formed with acetic acid or trihaloacetic acid, for example, trifluoroacetic acid), adipates, alginates, ascorbates, aspartates, benzoates, benzenesulfonates, bisulfates, borates, butyrates, citrates, camphorates, camphorsulfonates, cyclopentanepropionates, digluconates, dodecylsulfates, ethanesulfonates, fumarates, glucoheptanoates, glycerophosphates, hemisulfates, heptanoates, hexanoates, hydrochlorides, hydrobromides, hydroiodides, 2-hydroxyethanesulfonates, lactates, maleates, methanesulfonates, 2-naphthalenesulfonates, nicotinates, nitrates, oxalates, pectinates, persulfates, 3-phenylpropionates, phosphates, picrates, pivalates, propionates, salicylates, succinates, sulfates (such as those formed with sulfuric acid), sulfonates (such as those mentioned herein), tartrates, thiocyanates, toluenesulfonates, undecanoates, and the like.

Exemplary basic salts include ammonium salts, alkali metal salts such as sodium, lithium, and potassium salts, alkaline earth metal salts such as calcium and magnesium salts, salts with organic bases (for example, organic amines) such as benzathines, dicyclohexylamines, hydrabamines, N-methyl-D-glucamines, N-methyl-D-glucamides, t-butyl amines, and salts with amino acids such as arginine, lysine and the like.

Prodrugs and solvates of the compounds of the invention are also contemplated herein. The term “prodrug”, as employed herein, denotes a compound which, upon administration to a subject, undergoes chemical conversion by metabolic or chemical processes to yield a compounds described herein or a salt and/or solvate thereof.

All stereoisomers of the present compounds, including enantiomeric and diastereomeric forms, are contemplated within the scope of this invention. Individual stereoisomers of the compounds of the invention may, for example, be substantially free of other isomers, or may be admixed, for example, as racemates or with all other, or other selected, stereoisomers. The chiral centers of the present invention can have the S or R configuration as defined by the IUPAC 1974 Recommendations.

The preparations of the invention are administered in effective amounts. An effective amount is that amount of a pharmaceutical preparation that alone, or together with further doses, stimulates the desired response. In the case of treating a disorder or condition that is associated with abnormal PDE activity and/or abnormal levels of cAMP, desired response is reducing the onset, stage or progression of the abnormal PDE activity and/or levels of cAMP and associated effects. This may involve only slowing the progression of the damage temporarily, although more preferably, it involves halting the progression of the damage permanently. An effective amount for treating abnormal PDE activity and/or cAMP levels is that amount that alters (increases or reduces) the amount or level of PDE activity and/or cAMP level, when the cell or subject is a cell or subject with a PDE-associated disease or disorder, with respect to that amount that would occur in the absence of the active compound.

The invention involves, in part, the administration of an effective amount of a PDE-modulating compound of the invention. The PDE-modulating compounds of the invention are administered in effective amounts. Typically effective amounts of a PDE-modulating compound will be determined in clinical trials, establishing an effective dose for a test population versus a control population in a blind study. In some embodiments, an effective amount will be that amount that diminishes or eliminates a PDE-associated disease or disorder and its effects in a cell, tissue, and/or subject. Thus, an effective amount may be the amount that when administered reduces the amount of cell and or tissue damage and/or cell death from the amount that would occur in the subject or tissue without the administration of a PDE-modulating compound of the invention.

The pharmaceutical compound dosage may be adjusted by the individual physician or veterinarian, particularly in the event of any complication. A therapeutically effective amount typically varies from 0.01 mg/kg to about 1000 mg/kg, preferably from about 0.1 mg/kg to about 200 mg/kg, and most preferably from about 0.2 mg/kg to about 20 mg/kg, in one or more dose administrations daily, for one or more days. It will be recognized by those of skill in the art that some of the PDE-modulating compounds may have detrimental effects at high amounts. Thus, an effective amount for use in the methods of the invention may be optimized such that the amount administered results in minimal negative side effects and maximum PDE modulation.

The absolute amount will depend upon a variety of factors, including the material selected for administration, whether the administration is in single or multiple doses, and individual subject parameters including age, physical condition, size, weight, and the stage of the disease or disorder. These factors are well known to those of ordinary skill in the art and can be addressed with no more than routine experimentation.

Alternative drug therapies are known to those of ordinary skill in the art and are administered by modes known to those of skill in the art. The drug therapies are administered in amounts that are effective to achieve the physiological goals (to reduce symptoms and damage from a PDE-associated disease or disorder in a subject, e.g. cell damage and/or cell death), in combination with the pharmaceutical compounds of the invention. Thus, it is contemplated that the alternative drug therapies may be administered in amounts which are not capable of preventing or reducing the physiological consequences of the PDE-associated disease and/or disorder when the drug therapies are administered alone, but which are capable of preventing or reducing the physiological consequences of a PDE-associated disease and/or disorder when administered in combination with one or more PDE-modulating compounds of the invention.

Diagnostic tests known to those of ordinary skill in the art may be used to assess the level of PDE activity and/or levels of cAMP in a subject and the effects thereof, and to evaluate a therapeutically effective amount of a pharmaceutical compound administered. Examples of diagnostic tests are set forth below. A first determination of PDE activity, level of cAMP, and/or the effects thereof in a cell and/or tissue may be obtained using one of the methods described herein (or other methods known in the art), and a second, subsequent determination of the level of PDE activity or level of cAMP. A comparison of the PDE activity and/or cAMP level and/or the effects thereof on the subject at the different time points may be used to assess the effectiveness of administration of a pharmaceutical compound of the invention as a prophylactic or an active treatment of the PDE-associated disease or disorder. Family history or prior occurrence of a PDE-associated disease or disorder, even if the PDE-associated disease or disorder is absent in a subject at present, may be an indication for prophylactic intervention by administering a pharmaceutical compound described herein to reduce or prevent abnormal PDE activity and/or abnormal levels of cAMP.

An example of a method of diagnosis of abnormal PDE activity and/or abnormal levels of cAMP that can be performed using standard methods such as, but not limited to: imaging methods, electrophysiological methods, blood tests, and histological methods. Additional methods of diagnosis and assessment of PDE-associated disease or disorders and the resulting cell death or damage are known to those of skill in the art.

In addition to the diagnostic tests described above, clinical features of PDE-associated diseases and/or disorders can be monitored for assessment of PDE activity following onset of a PDE-associated disease or disorder. These features include, but are not limited to: assessment of the presence of cell damage, cell death, neuronal cell lesions, brain lesions, organ lesions, vascular damage, blood abnormalities, sugar processing abnormalities, and behavioral abnormalities. Such assessment can be done with methods known to one of ordinary skill in the art, such as behavioral testing, blood testing, and imaging studies, such as radiologic studies, CT scans, PET scans, etc.

The pharmaceutical compounds of the invention may be administered alone, in combination with each other, and/or in combination with other drug therapies that are administered to subjects with PDE-associated diseases or disorders.

In some embodiments the PDE-inhibiting compound is administered in combination with an additional drug for treating a PDE-associated disease or disorder. For example, selective PDE4 inhibitors or selective PDE7 inhibitors or dual PDE4-PDE7 inhibitor compounds described herein, may be administered alone or in combination with other suitable therapeutic agents useful in treating immune and inflammatory disorders such as: immunosuppressants such as, cyclosporins (e.g., cyclosporin A), anti-IL-1 agents, such as Anakinra, the IL-1 receptor antagonist, CTLA4-Ig, antibodies such as anti-ICAM-3, anti-IL-2 receptor (Anti-Tac), anti-CD45RB, anti-CD2, anti-CD3, anti-CD4, anti-CD80, anti-CD86, monoclonal antibody OKT3. agents blocking the interaction between CD40 and CD154, such as antibodies specific for CD40 and/or CD154 (i.e., CD40L), fusion proteins constructed from CD40 and CD154 (CD401 g and CD8-CD154), interferon beta, interferon gamma, methotrexate, FK506 (tacrolimus, Prograf), rapamycin (sirolimus or Rapamune)mycophenolate mofetil, leflunomide (Arava), azathioprine and cyclophosphamide, inhibitors, such as nuclear translocation inhibitors, of NF-kappa B function, such as deoxyspergualin (DSG), non-steroidal antiinflammatory drugs (NSAIDs) such as ibuprofen, cyclooxygenase-2 (COX-2) inhibitors such as celecoxib (Celebrex) and rofecoxib (Vioxx), or derivatives thereof, steroids such as prednisone or dexamethasone, gold compounds TNF-.alpha. inhibitors such as tenidap, anti-TNF antibodies or soluble TNF receptor such as etanercept (Enbrel), inhibitors of p-38 kinase such as BIRB-796, RO-3201195, VX-850, and VX-750, beta-2 agonists such as albuterol, levalbuterol (Xopenex), and saltmeterol (Screvent), inhibitors of leukotriene synthesis such as montelukast (Singulair) and zariflukast (Accolate), and anticholinergic agents such as ipratropium bromide (Atrovent). PDE4 inhibitors such as Arofyline, Cilomilast, Roflumilast, C-11294A, CDC-801, BAY-19-8004, Cipamfylline, SCH351591, YM-976, PD-189659, Mesiopram, Pumafentrine, CDC-998, IC-485, and KW-4490, PDE7 inhibitors such as IC242, (Lee, et. al. PDE7A is expressed in human B-lymphocytes and is up-regulated by elevation of intracellular cAMP. Cell Signalling, 14, 277-284, (2002)) and also include compounds disclosed in the following patent documents: WO 0068230, WO 0129049, WO 0132618, WO 0134601, WO 0136425, WO 0174786, WO 0198274, WO 0228847, U.S. Provisional Application Ser. No. 60/287,964, and U.S. Provisional Application Ser. No. 60/355,141 anti-cytokines such as anti-IL-1 mAb or IL-1 receptor agonist, anti-IL-4 or IL-4 receptor fusion proteins and PTK inhibitors such as those disclosed in the following U.S. Patents and Applications, incorporated herein by reference in their entirety: U.S. Pat. Nos. 6,235,740, 6,239,133, U.S. application Ser. No. 60/065,042, filed Nov. 10, 1997, U.S. application Ser. No. 09/173,413, filed Oct. 15, 1998, and U.S. Pat. No. 5,990,109.

### REFERENCES FOR DETAILED DESCRIPTION OF THE INVENTION

1. Bryne, S. M. And Hoffman, C. S., J. Cell. Sci. 105, (1993); p. 1095-1100.
2. Hoffman, C. S., Biochem. Soc. Trans., (2005); 33 (Pt 1): p. 257-60.
3. Hoffman, C. S., Eukaryotic Cell, (2005); 4: p. 495-503.
4. D′ souza et al., Mol. Cell. Biol., (2001); 21(9): p. 3179-91.
5. D′ souza, C. A. And Heitman, J., FEMS Microbiol. Rev., (2001); 25(3): p. 349-64.
6. Lengeler et al., Microbiol. Mol. Biol. Rev., (2000); 64(4): p. 746-85.
7. Thevelein, J. M. et al., Enzyme Microb. Technol., (2000); 26(9-10): p. 819-825.
8. Thevelein, J. M. And De Winde, J. H., Mol. Microbiol., (1999); 33(5): p. 904-18.
9. Nikawa et al., Genes Dev., (1987); 1(9): p. 931-7.
10. Wang, L. et al., Genetics, (2005); 171: p. 1523-33.
11. Ma, P. et al., Mol. Biol. Cell, (1999); 10(1): p. 91-104.
12. Ivey, F. D. and Hoffman, C. S., Proc. Natl. Acad. Sci. USA, (2005); 102(17): p. 6108
13. Landry, S, and Hoffman, C. S., Genetics, (2001); 157(3): p. 1159-68.
14. Landry, S. et al., Genetics, (2000); 154(4): p. 1463-1471.
15. Nocero, M. et al., Genetics, (1994); 138(1): p. 39-45.
16. Welton, R. M. and Hoffman, C. S., Genetics, (2000); 156: p. 513-21.
17. Hoffman, C. S, and Winston, F., Genetics, (1990); 124(4): p. 807-16.
18. Devoti, J. et al., Embo. J., (1991); 10(12): p. 3759-68.
19. Stiefel, J. et al., Eukaryotic Cell, (2004); 3(3): p. 610-619.
20. Wach et al., Yeast, (1997); Sep. 15; 13(11): p. 1065-75.
21. Hoffman, C. S. and Winston, F., Genes Dev., (1991); 5(4): p. 561-71.
22. Jin, M. et al., Genetics, (1995); 140(2): p. 457-67.
23. Schadick, et al., Eukaryot. Cell, (2002); 1(4): p. 558-67.
24. Mochizuki, N. and Yamamoto, M., Mol. Gen. Genet., (1992); 233(1-2): p. 17-24.
25. Kiely et al., Genetics, (2000); 154(2), p. 599-607.
26. Janoo et al., Genetics, (2001); 157(3), p. 1205-15.

### EXAMPLES

The examples below are non-limiting and are merely representative of various aspects and features of the presently disclosed embodiments.

### Example 1

### Construction of a Recombinant Fission Yeast Strain Capable of Reporting Changes in cAMP Concentration

Translational fusions carrying the fbp1 promoter fused to the S. pombe ura4 and the E. coli lacZ reporter genes were prepared and used to monitor the cell's ability to detect glucose. See Hoffman, C. S. and F. Winston, Genetics, 1990, 124(4): p. 807-16. These constructs were integrated in single copy into the S. pombe genome, creating stable reporters of fbp1 transcription.

Fission yeast strains were spotted onto yeast extract agar supplemented with 2% casamino acids (YEA medium) and grown overnight. PDE activity was then assessed by replica plating the cells onto either YEA medium, synthetic complete (SC) solid medium containing 8% glucose and 0.4 g/L 5-fluorourotic acid (5FOA medium), or SC medium containing 8% glucose with no uracil (SC-Ura medium). For details, including β-galactosidase and cAMP assays, see Wang et al., Genetics, 2005, 171, p. 1523-33. The results are shown in FIG. 1 and Table 1.

The cgs2-s1 and cgs2-s4 PDE gene mutations were isolated based on their ability to confer 5FOA-resistant growth to a strain carrying a mutation that prevented adenylate cyclase stimulation, leaving strains lacking adenylate cyclase 5FOA-sensitive (FIG. 1). In addition, the PDE mutations differentially suppress the loss of the gpa2 gene (Table 1; compare gpa2,Δcgs2-s1 and gpa24 cgs2-2), demonstrating that different reductions in PDE activity can be required to confer 5FOA-resistance depending upon the genetic background of the strain. In effect, different mutations that affect the generation of cAMP can be used to “tune” the cells such that their growth behavior reflects the level of PDE activity. See Wang et al., Genetics, 2005, 171(4): p. 1523-33 for description of the mutations.

TABLE 1
Phenotypes associated with fbp1 reporters
in different genetic backgrounds.

| Strain | βgal level repressed | 5FOA growth | basal cAMP level |
|---|---|---|---|
| Wild type | 10 | ++ | 3.6 |
| git3Δ (GPCR) | 925 | − | 1.7 |
| gpa2Δ (Gα) | 1400 | − | 2.0 |
| git5Δ (Gβ) | 1050 | − | 3.2 |
| git11Δ (GΔ) | 300 | − | ND |
| gpa2Δ cgs2-s1 | 480 | − | ND |
| gpa2Δ cgs2-2 | 10 | ++ | ND |
| git3Δ cgs2-s1 | 30 | ++ | 4.4 |
| git3Δ cgs2-2 | 4 | ++ | 11.6 |
| cgs2-s1 | 4 | ++ | 4.1 |
| cgs2-2 | 7 | ++ | 13.3 |
| gpa2^{R176H} | 5 | ++ | 6.9 |

### Example 2

### Quantification of cAMP Levels Using Recombinant Fission Yeast

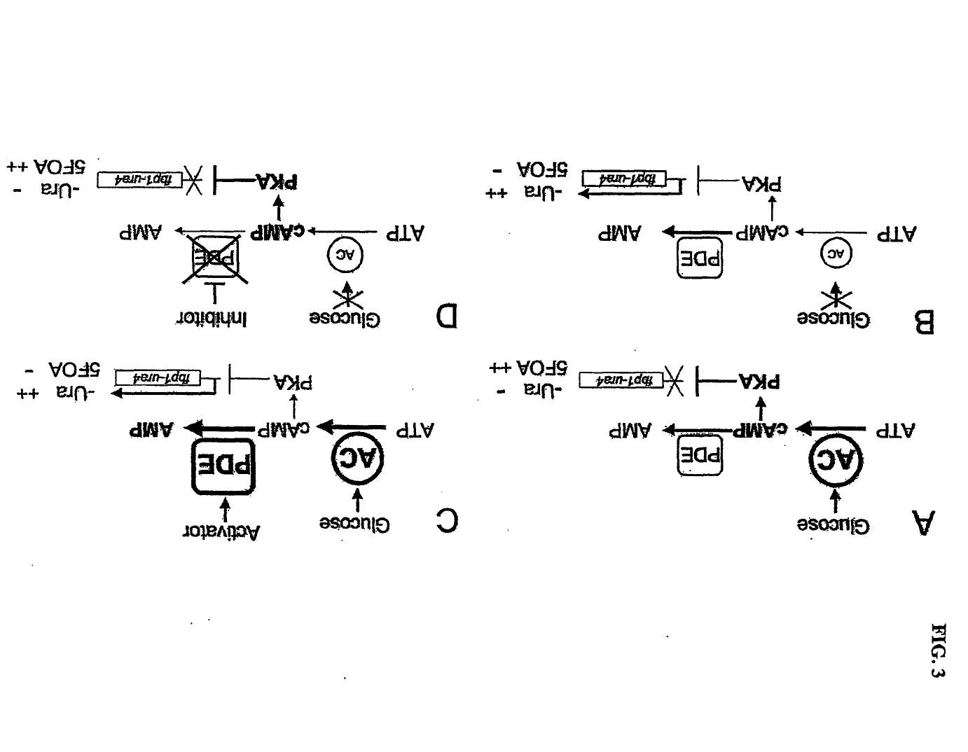

Wild type and two mutant strains (git1-1 and git2-7) having reduced cAMP levels were incubated overnight (18-24 hours) in EMM medium containing 5 mM cAMP to repress transcription of an fbp1-lacZ reporter construct from the fbp1 promoter and consequently repress β-galactosidase activity. Cyclic AMP was washed out by tranferring the cells to EMM without cAMP at time 0. Washout of cAMP stimulated expression of β-galactosidase to an extent depending on the cellular machinery controlling cAMP levels. The results are shown in FIG. 2. The relative sensitivity of the mutant strains to 5FOA is shown in Table 2. The git1-1 strain, which was considerably more sensitive to 5FOA, yields the highest β-galactosidase activity after washout of cAMP in FIG. 2, demonstrating a semi-quantitative correlation between cAMP metabolism and cell growth in the presence of 5FOA.

TABLE 2
Growth of S. pombe strains in 5FOA-containing medium
correlates with effect of mutations on fbp1 expression.

Fold increase

| Genotype | −cAMP | +cAMP |
|---|---|---|
| Wild type | 146 | 122 |
| git2-7 | 10 | 86 |
| git1-1 | 3.6 | 80 |

The fold increase in cell number is shown following 24 hours growth after transfer to 0.4 g/L 5FOA in the presence or absence of 5 mM cAMP.

### Example 3

### Use of a Recombinant Fission Yeast For High Throughput Screening for Chemical Inhibitors of PDE

Two 5FOA-sensitive strains are pregrown in the presence of 5 mM cAMP to repress transcription from the fbp1 promoter. Both strains possess the fbp1-ura4 and fbp1-lacZ reporter constructs. The experimental strain also expresses PDE4A1 in place of the yeast PDE. The control strain expresses the endogenous yeast PDE. Each strain is put individually into 384 well microtiter plates in a growth medium that contains 5FOA and 8% glucose, but no exogenous cAMP. These plates are used to screen a chemical library using robots that pin various compounds into the individual wells. If a compound has no effect on PDE activity or on any component of the yeast cAMP pathway, the cells of both strains deplete their cAMP leading to increased fbp1-ura4 transcription, which inhibits growth in the presence of 5FOA. If a compound stimulates cAMP production by targeting a component of the yeast cAMP pathway or inhibits fbp1-ura4 expression in a cAMP-independent manner, both strains display enhanced 5FOA-resistant growth to a similar degree. If a compound is an inhibitor of the exogenous PDE, the cAMP levels rise in the experimental strain, but not in the control strain, leading to differential 5FOA-resistant growth. Growth of the experimental and control strains are measured by measuring optical density. The effect of a compound is independently verified by measuring β-galactosidase expression from the fbp1-lacZ reporter in the experimental strain and by direct measurement of cAMP levels.

### Example 4

### A Fission Yeast-Based High Throughput Screen to Identify Chemical Modulators of cAMP Phosphodiesterase

Described herein is a fission yeast-based platform to detect compounds that either inhibit or activate heterologously-expressed cAMP phosphodiesterases (PDEs) that is suitable for high throughput drug screening. PDEs comprise a superfamily of enzymes that serve as drug targets in a variety of human diseases. The utility of this system is demonstrated by the construction and characterization of strains that express mammalian PDE2A, PDE4A, PDE4B, and PDE8A and respond appropriately to treatment with known PDE2A and PDE4 inhibitors. High throughput drug screens of two bioactive compound libraries were successfully conducted for PDE inhibitors using strains expressing PDE2A, PDE4A, PDE4B, and the yeast PDE Cgs2, demonstrating the ability of this system to determine PDE specificity through parallel screens of strains expressing distinct enzymes. The use of this platform to identify both chemical activators of PDEs, as well as genes that encode biological modulators of PDEs, which could serve as targets for future drug screens, is also discussed.

### INTRODUCTION

Cyclic AMP (cAMP) signaling pathways are employed by unicellular organisms and metazoan cells to transduce signals from a cell's surroundings to elicit appropriate responses. Unicellular organisms generally use this pathway to control metabolism and sexual development, often as a function of carbon source signaling. Mammalian cells produce cAMP signals in response to the detection of a variety of molecules including hormones, odorants, and neurotransmitters. This signaling pathway in mammals is complicated due to the presence of multiple cAMP-producing adenylyl cyclases and cAMP-destroying cAMP phosphodiesterases (PDEs)^{1, 2.}

There are 11 families of mammalian PDEs encoded by 21 genes, which produce more than 100 isoenzymes^{2, 3}. PDEs from the PDE4, PDE7, and PDE8 families specifically act on cAMP, PDEs from the PDE1, PDE2, PDE3, PDE10, and PDE11 families act on both cAMP and cGMP, while PDEs from the PDE5, PDE6, and PDE9 families act preferentially on cGMP. The presence of multiple PDE isoenzymes in various tissues complicates efforts to determine the relative roles of specific enzymes in any given biological process. Even so, chemical inhibitors of PDEs, and in some cases chemical activators, are seen as potential therapeutic compounds for the treatment of a variety of conditions including anxiety, depression, Alzheimer's disease, Parkinson's disease, Huntington's disease, schizophrenia, psychosis, sepsis, asthma, chronic obstructive pulmonary disease, pulmonary hypertension, renal disease, stroke, rhinitis, psoriasis, memory loss, chronic lymphocytic leukemia, prostate cancer, thyroid disease, male hypogonadism, cardiac disease, diabetes, obesity, multiple sclerosis, rheumatoid arthritis, penile erectile dysfunction, osteoporosis and cystic fibrosis²⁻⁹. Described here is an in vivo screen for identifying both chemical inhibitors and activators of cAMP PDEs using a simple growth assay in the fission yeast Schizosaccharomyces pombe.

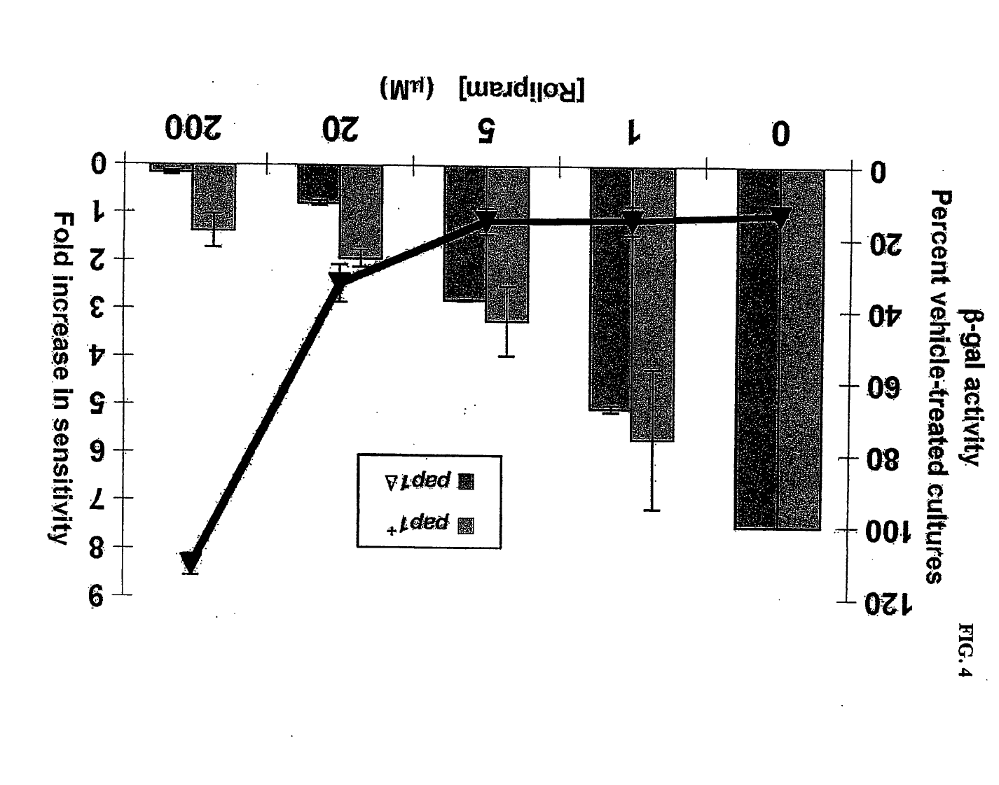

Previous studies on S. pombe glucose/cAMP signaling made use of two reporters whose expression is driven by the glucose-repressible fbp1⁺ promoter¹⁰. The fbp1-ura4 reporter places uracil biosynthesis under the control of the glucose/cAMP pathway, such that cells with high cAMP levels from glucose signaling cannot grow in medium lacking uracil (SC-ura), but do grow in medium containing the pyrimidine-analog 5-fluoro-orotic acid (5FOA), due to repression of the reporter (FIG. 3A). In contrast, cells with low cAMP levels from defects in glucose signaling grow in medium lacking uracil, but die in 5FOA medium, due to expression of the reporter (FIG. 3B). The second reporter, fbp1-lacZ, allows for easy quantitation of expression from the fbp1⁺ promoter. It is shown herein that strains expressing the mammalian enzymes PDE2A, PDE4A, PDE4B, and PDE8A produced functional PDEs whose activities affected the expression of these fbp1-driven reporters. In addition, reporter expression in PDE4A- and PDE4B-expressing strains was repressed by the PDE4 inhibitor rolipram, while reporter expression in a PDE2A-expressing strain was repressed by the PDE2A inhibitor erythro-9-(2-hydroxy-3-nonyl)adenine (EHNA). Successful high throughput drug screens for chemical inhibitors of the PDE2A, PDE4A, PDE4B, and yeast PDE Cgs2 have validated the utility of this platform. Also described, are additional capabilities of this screening platform to identify chemical activators of PDEs, as well as genes that encode biological activators or inhibitors of PDEs, which can serve as target proteins in future drug screens. The flexibility and versatility of this system demonstrate that the screen is an effective way to identify both chemical and biological modulators of PDEs from a variety of organisms.

### Methods

Yeast strains used are listed in Table 3. For the values in Table 3 β-galactosidase activity was determined from two to three independent exponential phase cultures. The average±SD represents specific activity per milligram of soluble protein.

TABLE 3
β-galactosidase activity from fbp1-lacZ
expression in gpa2⁻ mutant strains

| Strain | PDE | β-galactosidase activity |
|---|---|---|
| CHP861 | Cgs2⁺ | 2537 ± 292 |
| LWP364 | PDE2A | 331 ± 28 |
| CHP1098 | PDE4A | 1383 ± 269 |
| DIP72 | PDE4B | 825 ± 70 |
| DDP13 | PDE8A | 473 ± 139 |
| LWP98 | Cgs2-2 | 40 ± 4 |

Methods for the growth and transformation of fission yeast have been previously described¹⁹. The murine PDE genes were amplified by PCR using oligonucleotides containing approximately 60 nt of sequence flanking the S. pombe cgs2⁺ gene to direct homologous recombination to this locus. The recipient strain carries a ura4⁺-marked disruption of cgs2⁺²⁹ (also referred to as pde1⁺) to allow for 5FOA-counterselection for candidate transformants. PCR was used to confirm the homologous integration events. Subsequent strains were constructed by standard genetic crosses and tetrad dissection to introduce the fbp1-lacZ and fbp1-ura4 reporters, as well as the pap1 Δ allele.

β-galactosidase assays and characterization of 5FOA-sensitivity were carried out as previously described¹⁰. cAMP assays were performed on exponential phase cells grown in EMM complete medium (3% glucose), using the Assay Designs cAMP EIA kit, according to manufacturer's instructions (Assay Designs, Ann arbor, MI).

High throughput drug screens were carried out at the Broad Institute's Chemical Biology Program screening facility (Broad Institute, Cambridge, Mass.). Depending upon the strain, cultures were pregrown to exponential phase in EMM complete medium containing from 0.5 to 2.5 mM cAMP to repress fbp1-ura4 transcription. Cells were collected by centrifugation, resuspended in 5FOA medium, and 25 μl were transferred to 384-well microtiter dishes (untreated, with flat clear bottoms) that had been pre-filled with 25 μl 5FOA medium and pre-pinned with 100 nl of compounds (stock solutions were generally 10 mM) from a subset of the Prestwick Bioactive and the Microsource Spectrum compound libraries. Starting cell concentrations ranged from 0.5×10⁵ to 4×10⁵ cells/ml depending on the screening strain. As appropriate, control plates received either 100 nl 10 mM rolipram or DMSO. Other positive control dishes contained 5 mM cAMP in the 5FOA medium. Cultures were grown for 48 hours at 30° C., sealed in an airtight container with moist paper towels to prevent evaporation. Optical densities (OD₆₀₀) of cultures were measured using a microplate reader. Bioinformatic analysis of the results to determine composite Z scores was performed as previously described^{30, 31}.

### Results

To develop yeast strains whose growth behaviors could serve as a reflection of the activity of heterologously-expressed PDEs, homologous recombination was used to replace the only S. pombe PDE gene, cgs2⁺, with each of four murine PDE genes, PDE2A, PDE4A, PDE4B, and PDE8A¹¹⁻¹³. Strains expressing these enzymes do not display the severe mating defect associated with the loss of PDE activity¹⁴, indicating that these PDEs are functional when expressed in S. pombe.

Next, strains were constructed that expressed the murine PDEs together with the fbp1-driven reporters, and carried mutant alleles of either the git3⁺ glucose receptor gene or the gpa2⁺Gα subunit gene, both of which were required for glucose detection, adenylyl cyclase activation, and transcriptional repression of the fbp1-ura4 and fbp1-lacZ reporters¹⁵⁻¹⁸. The relative level of reporter expression in these strains reflected the activity of the PDEs expressed. β-galactosidase activity in the gpa2⁻ mutant strains, as compared with similar strains expressing either the wild-type S. pombe Cgs2⁺ PDE or the frame-shifted, and presumably inactive, Cgs2-2 truncated PDE¹⁹ demonstrated that all four murine PDEs were active in S. pombe (Table 1). The relative level of PDE activity, as reflected by the degree to which β-galactosidase activity was elevated by the reduction in cAMP levels, was Cgs2⁺>PDE4A>PDE4B>PDE8A≈PDE2A>Cgs2-2. This order of activity was consistent with the ability of git3⁻ and gpa2⁻ mutations to confer 5FOA-sensitive (5FOA^{S}) growth to strains expressing the murine PDEs (see below).

The effect of known PDE inhibitors on the expression of the fbp1-lacZ fusion in murine PDE-expressing strains was tested. As seen in Table 4, rolipram, a PDE4 inhibitor, reduced β-galactosidase activity in PDE4A- and PDE4B-expressing cells, but not in Cgs2- or PDE8A-expressing cells. These results supported previous studies indicating that PDE8A was insensitive to rolipram. In addition, the PDE2A inhibitor EHNA reduced β-galactosidase activity expressed from a PDE2A strain (Table 4). For Table 4 β-galactosidase activity was determined from 3 to 4 independent exponential phase cultures. The average±SD represents specific activity per milligram of soluble protein. PDE8A was not able to be inhibited with dipyridamole, which has been shown to inhibit PDE8A¹², and this result may have been due to a permeability problem in the yeast.

TABLE 4
β-galactosidase activity in response to PDE inhibitor treatment

| | | β-galactosidase activity | | |
|---|---|---|---|---|
| Strain | PDE | Vehicle | 50 μM Rolipram | 100 μM Rolipram |
| CHP861 | Cgs2 | 1661 ± 121 | 1807 ± 446 | 1784 ± 429 |
| DDP26 | PDE4A | 998 ± 154 | 271 ± 30 | 162 ± 17 |
| DIP72 | PDE4B | 432 ± 170 | 32 ± 12 | 21 ± 7 |
| DDP13 | PDE8A | 241 ± 61 | 253 ± 46 | 237 ± 67 |
| LWP98 | Cgs2-2 | 23 ± 10 | 19 ± 9 | 20 ± 11 |

| | | β-galactosidase activity | | | |
|---|---|---|---|---|---|
| Strain | PDE | Vehicle | 5 μM EHNA | 20 μM EHNA | 200 μM EHNA |
| LWP367 | PDE2A | 587 ± 7 | 473 ± 19 | 197 ± 51 | 45 ± 3 |

In an effort to increase the sensitivity to PDE inhibitors, further experiments included examination of whether deleting pap1⁺, encoding a zinc finger transcriptional activator required for ABC transporter expression and whose overexpression confers staurosporine-resistance^{20, 21}, enhanced inhibition of PDE4A by rolipram. As shown in FIG. 4, PDE4A-expressing cells lacking pap1⁺ (pap1Δ), were more sensitive to rolipram than pap1⁺ cells. Moreover, pap1Δ strains that were 5FOA^{S} due to low cAMP levels maintained the 5FOA^{S} growth phenotype for longer periods of incubation than equivalent pap1⁺ strains. Such enhanced sensitivity to 5FOA is useful to help in the detection of compounds that confer 5FOA^{R} growth due to PDE inhibition.

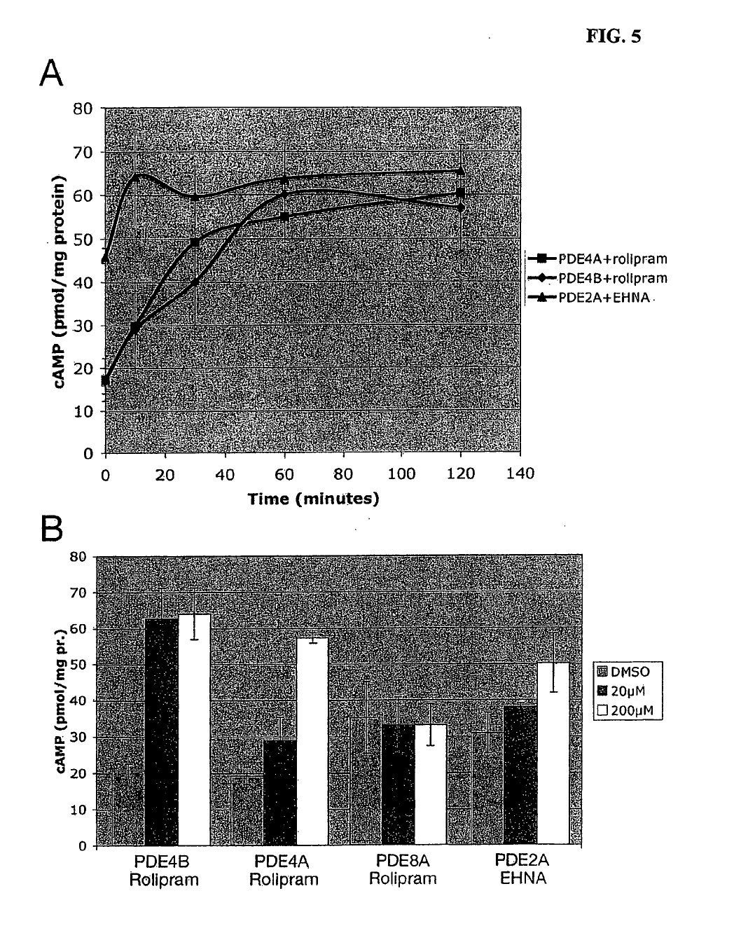

To determine if the effect of rolipram on PDE4-expressing cells and of EHNA on PDE2A-expressing cells was through inhibition of the heterologously-expressed PDEs, cAMP levels were measured before and after drug treatment. As shown in FIG. 5A, cAMP levels increased within 10 minutes of exposure to 200 μM inhibitor and reached peak levels within one hour. Additional experiments were performed to examine whether varying degrees of PDE inhibition could be detected by measuring cAMP levels at the one-hour time point in cells exposed to lower concentrations of inhibitor. FIG. 5B shows that PDE4A was only partially inhibited by 20 μM rolipram, while PDE4B was completely inhibited at this concentration, suggesting that PDE4B was more sensitive than PDE4A to rolipram in this system. Furthermore, cAMP levels in a strain expressing PDE8A were completely insensitive to rolipram treatment, consistent with previous studies of PDE8A¹², and also indicating that rolipram does not affect cAMP generation in fission yeast. Finally, PDE2A showed partial inhibition by EHNA at 20 μM as compared to 200 μM EHNA. Thus, PDE inhibition can be indirectly quantitated by measuring the effect of a compound on cAMP levels in target yeast strains.

Although the fbp1-lacZ reporter allowed for a measurement of PDE inhibition, the true power of this system is in the growth phenotype conferred by transcription of the fbp1-ura4 reporter. PDE inhibitors should restore 5FOA^{R} growth to strains possessing low basal cAMP levels by elevating cAMP levels to repress fbp1-ura4 transcription (FIG. 3D). Conversely, PDE activators should confer growth in SC-ura medium to strains possessing high cAMP levels by reducing cAMP levels to increase fbp1-ura4 transcription (FIG. 3C). As mentioned above, mutations in either the git3⁺ or gpa2⁺ genes were introduced into various PDE-expressing strains. While a gpa2⁻ mutant allele conferred 5FOA-sensitivity on PDE2A-, PDE4A-, PDE4B-, and PDE8A-expressing strains, only Cgs2- and PDE4A-expressing strains became 5FOA^{s} when carrying a mutant allele of git3⁺. These results are consistent with previous observations that loss of Gpa2 confers a greater defect in cAMP signaling than does loss of Git3^{10, 17, 18}, and that Cgs2 and PDE4A were more active than the other three PDEs in the strains used (Tables 3 and 4).

To determine whether the 5FOA growth phenotype could be exploited for high throughput drug screening, strains expressing PDE2A, PDE4A, PDE4B, or PDE8A were pre-grown in EMM medium containing cAMP and then transferred to 5FOA medium in 384 well microtiter plates in the presence or absence of cAMP. OD₆₀₀ measurements were taken after 48 hours incubation at 30° C. In each strain, the addition of cAMP to the growth medium restored 5FOA^{R} growth. Similar experiments in which 20 μM rolipram (final concentration) was pinned into 192 of the 384 wells, in place of cAMP addition to the medium, produced 5FOA^{R} growth in the PDE4A and PDE4B-expressing strains. For example, in a typical experiment with CHP1113 cells (PDE4B), the OD₆₀₀ of the rolipram-treated cultures was 1.28+/−0.07 while the OD₆₀₀ of the untreated wells was 0.18+/−0.02. When using CHP1098 cells (PDE4A), the OD₆₀₀ of the rolipram-treated cultures was 1.15+/−0.06, while the OD₆₀₀ of the untreated wells was 0.2+/−0.03. The Z factors (a statistical assessment of the quality of datasets used in high throughput screening²²) for these screens are 0.76 and 0.72, respectively, placing them well above the 0.5 minimum Z factor indicative of a robust screen.

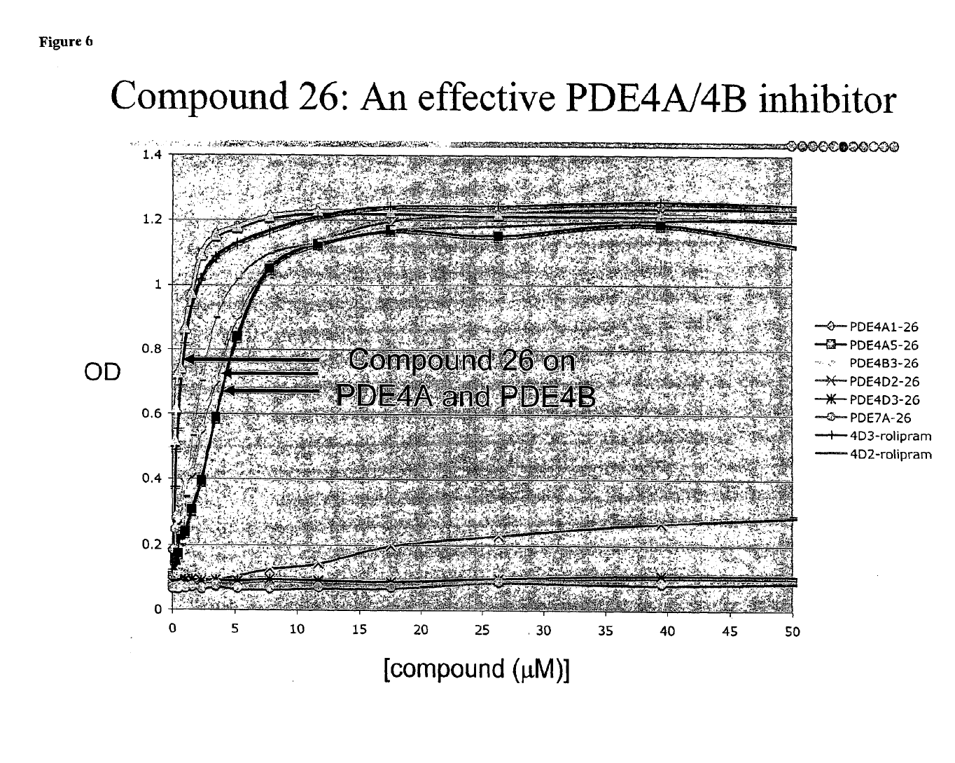

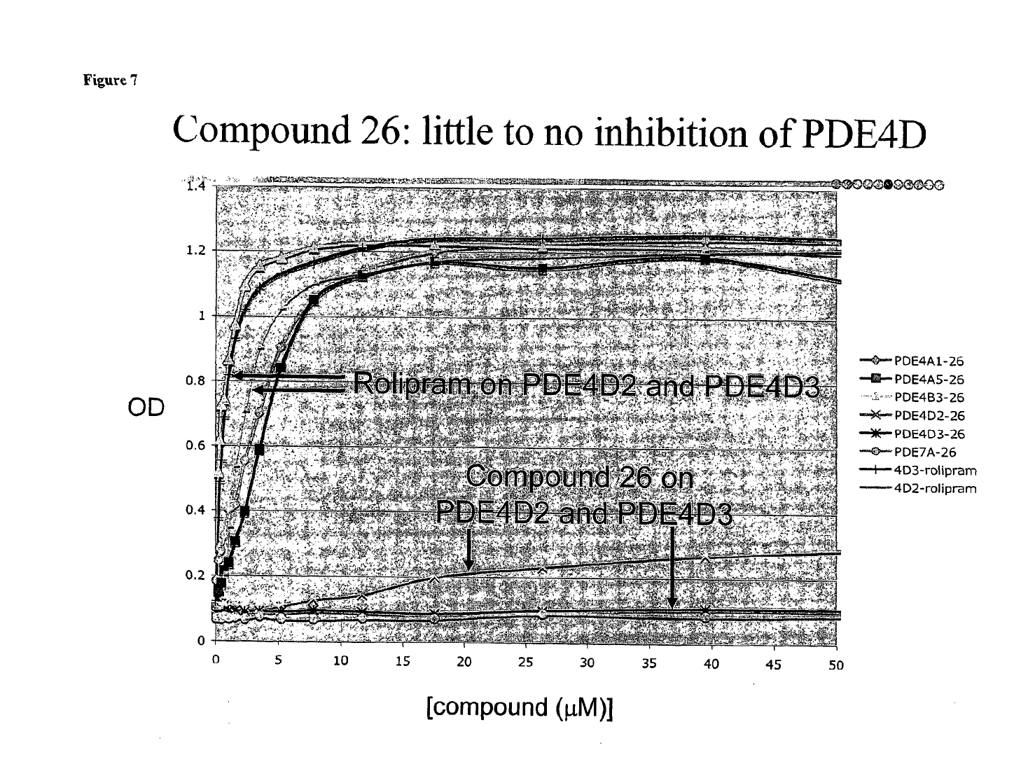

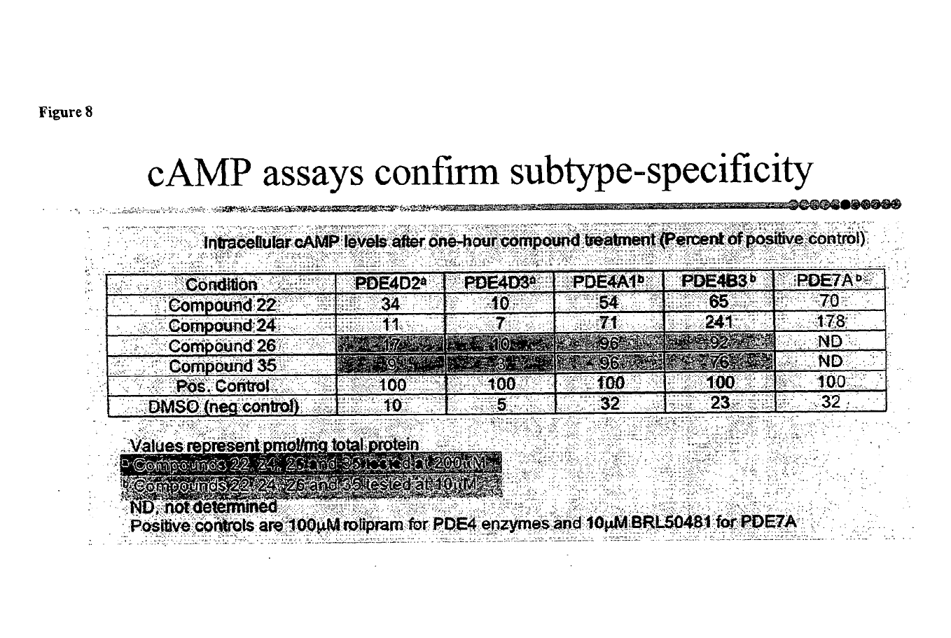

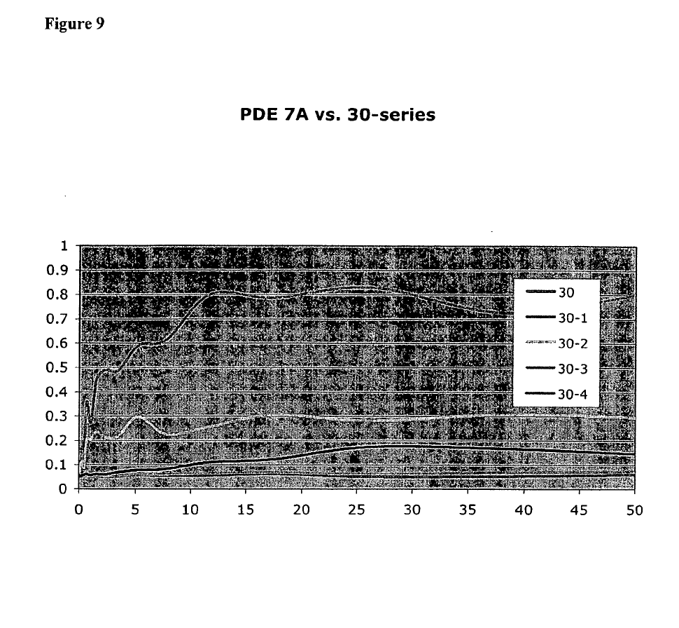

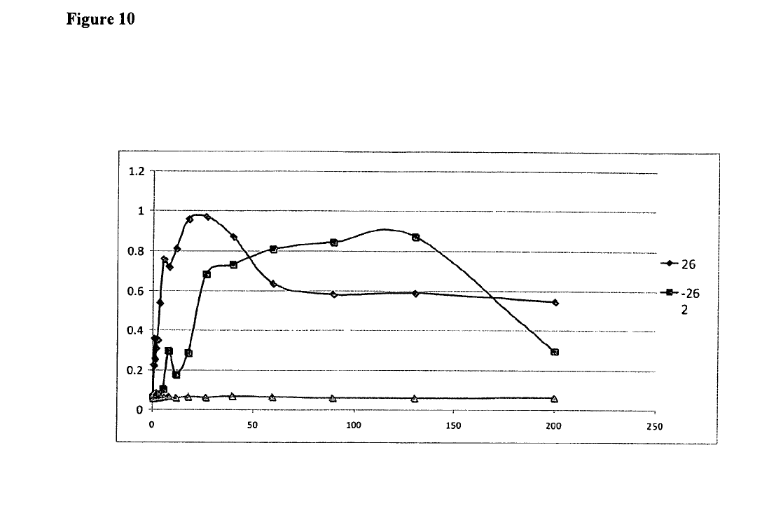

As a final test of the utility of this system, screening was performed on a pair of libraries containing 3,120 bioactive compounds, including known PDE inhibitors, using 5FOA^{S} strains expressing PDE2A, PDE4A, PDE4B, or Cgs2 for compounds that confer 5FOA^{R} growth. Duplicate plates were screened and compounds that confer 5FOA^{R} growth with composite Z scores of ≧8.53 (the cut-off used by the Broad Institute's Chemical Biology Program, where the screens were performed) were identified. FIG. 6 is a Venn diagram displaying the overlap of the compounds identified in these four screens. These results strongly validate this system for high throughput screening based on the relatively low number of compounds identified (from 0.8% to 3.2% of compounds tested per strain), the identification of known PDE4 inhibitors as PDE4-specific, and the identification of certain classes of compounds as PDE inhibitors as discussed below.

### DISCUSSION

This Example describes a novel fission yeast cell-based screening platform, amenable for high throughput drug screening to identify compounds that alter PDE activity. While a budding yeast system based on heat shock sensitivity of stationary phase cells has been previously reported²³, cells in that assay had to be exposed to 0.5 mM to 2 mM rolipram to detect an effect on PDE4B and was not amenable to a high throughput screening format^{24, 25}. In contrast, using these new assay methods has permitted successful screening of compound libraries at an average concentration of 20 μM to detect both known and previously unidentified PDE inhibitors (FIG. 6). This is a relatively inexpensive assay, and permits development of a large collection of strains expressing either mammalian cAMP-specific or dual-specificity PDEs. This platform is also used with PDEs from pathogens, whose inhibition may either kill the target pathogen or reduce virulence. Strains expressing a broad panel of PDEs are used to identify compounds possessing desirable specificity profiles to suggest the potential of individual compounds as candidate therapeutics. Moreover, because this platform identifies compounds based on stimulation of cell growth, it will not detect compounds that, while inhibiting PDEs in vitro, are too cytotoxic or cell-impermeable for therapeutic use. This is not the case for the majority of PDE assays, which are carried out in vitro on purified proteins or on protein extracts. In addition, this in vivo screening platform should be able to detect PDE inhibitors that may not be identified by in vitro screens. For example, compounds that prevent either intermolecular or intramolecular interactions required for enzyme formation would be overlooked in an in vitro assay on purified enzymes or protein extracts, yet should be identifiable in this assay.

High throughput screens against 3,120 bioactive compounds using strains expressing the yeast PDE Cgs2, or the murine PDEs 2A, 4A, and 4B identified a number of compounds that promote 5FOA^{R} growth, presumably by inhibiting the target PDEs to raise cAMP levels. These included the known PDE4 inhibitors rolipram and zardaverine, which only affected the PDE4A- and PDE4B-expressing strains. Other compounds identified in the screens are members of the coumarin, furocoumarin, and flavonoid families that are known to have PDE inhibitory properties (see review by Peluso, 2006²⁶). For example, the screens identified the furocoumarins trioxsalen, khellin, and visnagin, which are known PDE inhibitors^{27, 28}. In addition, the relative overlap of the compounds identified in each screen further validated this platform, but also indicate additional features. Candidates from the Cgs2 screen display the least overlap with candidates from the other three screens (FIG. 6), consistent with the fact that the murine PDEs are more closely related to each other than to Cgs2. Furthermore, a substantial number of compounds (18) inhibited both PDE4A and PDE4B, but not Cgs2 or PDE2A, consistent with the pharmacological grouping of PDE4A and PDE4B into the PDE4 family. On the other hand, there was unexpected amount of overlap of candidates from the PDE2A and PDE4B screens. As these target PDEs appear to be less active in fission yeast than Cgs2 and PDE4A (Table 3), some of these candidates may either have weakly reduced fbp1-driven transcription by a cAMP-independent manner or raised cAMP levels by stimulating adenylyl cyclase activity. This later option is consistent with the presence of diterpenoids in this group of compounds. However, as compounds with novel structures are identified, distinctions can be made among cAMP-independent effects, adenylyl cyclase activation and PDE inhibition by measuring cAMP levels in experimental and control strains as in FIG. 5B. Specifically, compounds that act in a cAMP-independent manner will not affect cAMP levels in any strains, while compounds that activate adenylyl cyclase will elevate cAMP levels in control strains that lack PDE activity. Finally, a number of compounds were detected in only one of each the four screens, lending support to this platform as a tool for identifying isoenzyme-specific inhibitors.

The ability to identify PDE inhibitors is based on the growth phenotype conferred by the cAMP-repressible fbp1-ura4 reporter. This system can also identify compounds that stimulate PDE activity to lower cAMP levels and increase fbp1-ura4 expression. PDE activators should confer Ura⁺ growth to strains whose high basal cAMP levels repress fbp1-ura4 expression in the absence of drug exposure (FIG. 3C). Finally, as yeast are capable of maintaining autonomously-replicating plasmids, one can screen cDNA libraries for genes that encode biological inhibitors or activators of target PDEs, which can serve as novel targets for high throughput drug screens. Thus, this screening platform can be used to identify novel PDE inhibitors and activators, as well as new ways to moderate cAMP signaling pathways in an effort to improve therapeutic approaches to treating a wide array of human diseases.

### REFERENCES FOR EXAMPLE 4

1. Kamenetsky, M. et al. J Mol Biol 362, 623-639 (2006).
2. Bender, A. T. & Beavo, J. A. Pharmacol Rev 58, 488-520 (2006).
3. Lerner, A. & Epstein, P. M. Biochem J393, 21-41 (2006).
4. Vasta, V., et al., Proc Natl Acad Sci USA 103, 19925-19930 (2006).
5. Dyke, H. J. & Montana, J. G. Expert Opin Investig Drugs 11, 1-13 (2002).
6. Boswell-Smith, et al., Br J Pharmacol 147 Suppl 1, S252-257 (2006).
7. O′Donnell, J. M. & Zhang, H. T. Trends Pharmacol Sci 25, 158-163 (2004).
8. Lugnier, C. Pharmacol Ther 109, 366-398 (2006).
9. Hebb, A. L. & Robertson, H. A. Curr Opin Pharmacol (2006).
10. Hoffman, C. S. & Winston, F. Genetics 124, 807-816 (1990).
11. Chemy, J. A., et al., Biochim Biophys Acta 1518, 27-35 (2001).
12. Soderling, S. H., et al., Proc Natl Acad Sci USA 95, 8991-8996 (1998).
13. Wu, A. Y., et al. J Biol Chem 279, 37928-37938 (2004).
14. DeVoti, J., et al., Embo J 10, 3759-3768 (1991).
15. Hoffman, C. S. Biochem Soc Trans 33, 257-260 (2005).
16. Ivey, F. D. & Hoffman, C. S. Proc Natl Acad Sci USA 102, 6108-6113 (2005).
17. Nocero, M., et al. Genetics 138, 39-45 (1994).
18. Welton, R. M. & Hoffman, C. S. Genetics 156, 513-521 (2000).
19. Wang, L., et al., Genetics 171, 1523-1533 (2005).
20. Toone, W. M. et al. Genes Dev 12, 1453-1463 (1998).
21. Toda, T., et al., Genes Dev 5, 60-73 (1991).
22. Zhang, J. H., et al., J Biomol Screen 4, 67-73 (1999).
23. Colicelli, J. et al. Proc Natl Acad Sci USA 88, 2913-2917 (1991).
24. Pillai, R., et al., Proc Natl Acad Sci USA 90, 11970-11974 (1993).
25. Atienza, J. M. & Colicelli, J. Methods 14, 35-42 (1998).
26. Peluso, M. R. Exp Biol Med (Maywood) 231, 1287-1299 (2006).
27. Duarte, J. et al. Gen Pharmacol 32, 71-74 (1999).
28. Bovalini, L. et al. Z Naturforsch [C] 42, 1009-1010 (1987).
29. Mochizuki, N. & Yamamoto, M. Mol Gen Genet. 233, 17-24 (1992).
30. Kim, Y. K. et al. J Am Chem Soc 126, 14740-14745 (2004).
31. Franz, A. K., et al., J Am Chem Soc 129, 1020-1021 (2007).

### Example 5

### Methods for Preparing Yeast Strains Containing Exogenous PDEs

These methods can be used to prepare fission yeast strains that lack endogenous cAMP PDEs and that include one or more exogenous PDE. Conditions to promote growth and to optimize cAMP levels for any specific strain generated may be determined using methods in the art and/or methods described herein.

This example provides protocols that have been and can be used to introduce PDE genes into the fission yeast. The resulting yeast strains are useful in screening methods and assays for cAMP PDE activators and inhibitors.

PDE genes were introduced into the fission yeast PDE gene locus (cgs2⁺) by PCR amplification of the gene to be introduced using oligonucleotides that contain sequences that flank the cgs2 gene. The PCR product was used to transform strain JZ666, which contains a ura4⁺-marked deletion of cgs2, which allowed for 5FOA-counterselection to identify colonies that have lost the ura4 gene due to its replacement by the PDE gene through homologous recombination. The host strain is homothallic (cells from the same strain are capable of mating with each other), however mating of this strain is defective due to the high cAMP levels conferred by the disruption of the cgs2 PDE gene. An initial screen for candidates that received a foreign PDE gene was carried out by either microscopic examination of cells growing on defined medium (Edinburgh minimal medium (EMM) for example) or by exposing plates to iodine vapors, which stain asci that are produced by mating. A second feature of reducing cAMP levels is that cells show improved survival in stationary phase. This was and can be screened for by microscopy or by replica plating colonies from plates that have been incubated for as much as one week to a fresh plate, and by examining the efficiency with which cells from individual colonies are able to grow and form new colonies. Candidate colonies from either method are further examined by PCR to detect the homologous recombination event that would introduce the foreign PDE gene into the cgs2⁺ locus.

Because homologous recombination is not as efficient in S. pombe as it is in budding yeast, an alternative strategy has also been employed to introduce PDE genes into the cgs2 locus. Rather than directly introducing the PCR product into the chromosomal locus, JZ666 cells were co-transformed with the PCR product and a linearized plasmid that carries the ura4-marked disruption of cgs2. By digesting the plasmid within the ura4 gene, homologous recombination between the plasmid and the PCR product was stimulated. The PDE gene recombines into the plasmid through the process of gap repair at a higher efficiency than seen for recombination into the chromosome. Cells carrying plasmids that express the PDE were identified as described above. Once the plasmid had been rescued to E. coli and a plasmid preparation was obtained, the plasmid was digested with one or two restriction enzymes to produce a fragment containing the PDE gene along with 500 to 2000 base pairs of cgs2 flanking sequences. This fragment was used to introduce the PDE gene into the cgs2 chromosomal locus in strain JZ666 by homologous recombination. This was more efficient than the direct transformation with a PCR product (described above) because this fragment possesses significantly more targeting sequences at its ends.

For the design of oligonucleotides for PCR, the 5′ end of each oligonucleotide should contain approximately 60 nucleotides from the following sequences that flank cgs2.

Forward targeting sequence (the final ATG
represents the Cgs2 START codon)

(SEQ ID NO: 1)

5′TCTCCACATTTCGAGCATCGTTTATCGTACCCTAAATCTACGGTAGTA
AATGTATGCTTGTAATAAATATGACGTCAACCGACATGTTTTTGTAGACT
AGTGCATGCACCGGAGATCTGTAACTCTCCATAAGCCTAGCCATG 3′
Reverse targeting sequence

(SEQ ID NO 2)

5′AAGCGAGGTACGATGAACTGGTAATGAAAAATAAAAAAAGGTAATAAT
TAATTGCTTTAGCATTCAATAATTAACAACAAAGTCAAAATTCCTCCAAC
AG 3′

As a specific example, to introduce the human PDE4D3 gene into the cgs2 locus, the following two oligonucleotides were used to PCR amplify PDE4D3 from a plasmid carrying this cDNA.

Forward oligonucleotide

(SEQ ID NO: 3)

TGTTTTTGTAGACTAGTGCATGCACCGGAGATCTGTAACTCTCCATAAGC
CTAGCCATGATGCACGTGAATAATTTTCCC
Reverse oligonucleotide

(SEQ ID NO: 4)

TAATAATTAATTGCTTTAGCATTCAATAATTAACAACAAAGTCAAAATTC
CTCCAACAGTTACGTGTCAGGAGAACGATC

This approach has been successfully used with the following PDE genes and is used with additional PDE genes.

Murine PDE1C4 (Genbank Accession number L76947)
Murine PDE2A (Genbank Accession number NM_{—}001008548)
Murine PDE3B (Genbank Accession number AF547435)
Murine PDE4A1 (Genbank Accession number NM_{—}019798)
Rat PDE4A5 (Genbank Accession number L27057)
Murine PDE4B3 (Genbank Accession number NM_{—}019840)
Human PDE4D3 (Genbank Accession number U50159)
Human PDE7A (Genbank Accession number L12052)
Murine PDE8A (Genbank Accession number BC132145)
Trypanosoma brucei PDEB 1 (Genbank Accession number AY028446)
Trypanosoma brucei PDEB2 (Genbank Accession number XM_{—}798722)
Trypanosoma cruzi PDEB 1 (Genbank Accession number AY099403)
Human PDE10A (Genbank Accession number NM_{—}006661)

The sequences of oligonucleotide primers used in the construction of the strains are provided in Table 5.

TABLE 5

| | |
|---|---|
| PDE gene | Accession |

| | |
|---|---|
| PDE1C4 | L76947 |
| Forward | CATGTTTTTGTAGACTAGTGCATGCACCGGAGATCTGTAACTCT |
| | CCATAAGCCTAGCCATGGAGTCTCCAACCAAGGAAA (SEQ ID NO: 5) |
| Reverse | AATGAAAAATAAAAAAAGGTAATAATTAATTGCTTTAGCATTCA |
| | ATAATTAACAACAAAGTCAAAATTCCTCCAACAGTTATCC |
| | GTAGTCTCCTGGCAAG (SEQ ID NO: 6) |
| PDE2A | NM_001008548 |
| Forward | ACATGTTTTTGTAGACTAGTGCATGCACCGGAGATCTGTAACTCTC |
| | CATAAGCCTAGCCATGGGGCAGGCATGCGGCCAC (SEQ ID NO: 7) |
| Reverse | ATAATTAATTGCTTTAGCATTCAATAATTAACAACAAAGTCAAAA |
| | TTCCTCCAACAGTCAGCCCTCGAGGCTGCAGCAGC (SEQ ID NO: 8) |
| PDE3B | AF547435 |
| Forward | ACATGTTTTTGTAGACTAGTGCATGCACCGGAGATCTGTAACTCT |
| | CCATAAGCCTAGCCATGAGGAAAGACGAGCGCGAG (SEQ ID NO: 9) |
| Reverse* | TAATAATTAATTGCTTTAGCATTCAATAATTAACAACAAAGTCAA |
| | AATTCCTCCAACAGAGGCCTGAATTCCTCGAGGTC (SEQ ID NO: 10) |
| PDE4A1 | NM_019798 |
| Forward | ACATGTTTTTGTAGACTAGTGCATGCACCGGAGATCTGTAACTCTC |
| | CATAAGCCTAGCCATGCCTCTGGTTGACTTCTTC (SEQ ID NO: 11) |
| Reverse | AAATTAAAAAAAAAAAATAAAAATATAATGAATATATGACCATGA |
| | CCCTGGGATGCTATTAGGCAGGGTCTCCACCTGAC (SEQ ID NO: 12) |
| PDE4A5 | L27057 |
| Forward | ATGTTTTTGTAGACTAGTGCATGCACCGGAGATCTGTAACTCTCCA |
| | TAAGCCTAGCCATGAGCCATGGAGCCTCCGGCCG (SEQ ID NO: 13) |
| Reverse | AATAATTAATTGCTTTAGCATTCAATAATTAACAACAAAGTCAAAA |
| | TTCCTCCAACAGTCAGGCAGGGTCTCCGCCTGAC (SEQ ID NO: 14) |
| PDE4B3 | NM_019840 |
| Forward | GACATGTTTTTGTAGACTAGTGCATGCACCGGAGATCTGTAACTC |
| | TCCATAAGCCTAGCCATGACAGCAAAAAATTCTCC (SEQ ID NO: 15) |
| Reverse | ATTAAAAAAAAAAAATAAAAATATAATGAATATATGACCATGAC |
| | CCTGGGATGCTACTAAACTCTAGATATTCAACAGGC (SEQ ID NO: 16) |
| PDE4D3 | U50159 |
| Forward | TGTTTTTGTAGACTAGTGCATGCACCGGAGATCTGTAACTCTCCA |
| | TAAGCCTAGCCATGATGCACGTGAATAATTTTCCC (SEQ ID NO: 3) |
| Reverse | TAATAATTAATTGCTTTAGCATTCAATAATTAACAACAAAGTCAA |
| | AATTCCTCCAACAGTTACGTGTCAGGAGAACGATC (SEQ ID NO: 4) |
| PDE7A | L12052 |
| Forward* | ACATGTTTTTGTAGACTAGTGCATGCACCGGAGATCTGTAACTCT |
| | CCATAAGCCTAGCCGGACGGCCTCCGAAACCATG (SEQ ID NO: 17) |
| Reverse | AAAAAGGTAATAATTAATTGCTTTAGCATTCAATAATTAACAAC |
| | AAAGTCAAAACCTTATGATAACCGATTTTCCTGAGG (SEQ ID NO: 18) |
| PDE8A | BC132145 |
| Forward | AAATATGACGTCAACCGACATGTTTTTGTAGACTAGTGCATGCACC |
| | GGAGATCTGTAACTCTCCATAAGCCTAGATGGGC (SEQ ID NO: 19) |
| Reverse | GGTAATAATTAATTGCTTTAGCATTCAATAATTAACAACAAAGTCA |
| | AAATTCCTCCAACAGGCAGCTCTGGCTAACAGTG (SEQ ID NO: 20) |
| T. brucei | |
| PDEB1 | AY028446 |
| Forward | ATGTTTTTGTAGACTAGTGCATGCACCGGAGATCTGTAACTCTCCAT |
| | AAGCCTAGCCATGTTCATGAACAAGCCCTTTGG (SEQ ID NO: 21) |
| Reverse* | AGGTAATAATTAATTGCTTTAGCATTCAATAATTAACAACAAAGTCA |
| | AAATTCCTCCAACAGTCGAGGCTGATCAGCGGG (SEQ ID NO: 22) |
| T. brucei | |
| PDEB2 | XM_798722 |
| Forward | CATGTTTTTGTAGACTAGTGCATGCACCGGAGATCTGTAACTCTCCA |
| | TAAGCCTAGCCATGACACACAACGGTGGTCGTC (SEQ ID NO: 23) |
| Reverse | AGGTAATAATTAATTGCTTTAGCATTCAATAATTAACAACAAAGTCA |
| | AAATTCCTCCAACAGTCGAGGCTGATCAGCGGG (SEQ ID NO: 24) |
| T. cruzi | |
| PDEB1 | AY099403 |
| Forward | ACATGTTTTTGTAGACTAGTGCATGCACCGGAGATCTGTAACTCTCC |
| | ATAAGCCTAGCCATGGGGCAGGCATGCGGCCAC (SEQ ID NO: 25) |
| Reverse | AGGTAATAATTAATTGCTTTAGCATTCAATAATTAACAACAAAGTCAA |
| | AATTCCTCCAACAGTCGAGGCTGATCAGCGGG (SEQ ID NO: 26) |
| Homo | |
| sapiens | |
| PDE10A | NM_006661 |
| Forward* | GACATGTTTTTGTAGACTAGTGCATGCACCGGAGATCTGTAACTCTC |
| | CATAAGCCTAGCCGGCACCAAAATCAACGGGAC (SEQ ID NO: 27) |
| Reverse* | GTAATAATTAATTGCTTTAGCATTCAATAATTAACAACAAAGTC |
| | AAAATTCCTCCAACAGTTATT AGGACAAGGCTGGTG (SEQ ID NO: 28) |

*Oligonucleotide is designed to prime off of the vector sequence rather than the sequence of the P

Once the PDE gene was introduced into the cgs2 locus, drug screening strains were constructed by standard genetic crosses with strains that contain the following genetic features.

1. fbp1-ura4 fusion: This is the reporter that produces the cAMP-dependent growth characteristics.
2. fbp1-lacZ fusion: While not necessary for high throughput screening, this reporter allows easy quantitation of expression from the fbp1 promoter, which can be useful for characterizing the effect of adding candidate compounds or cAMP or cGMP to the growth medium (see below).
3. pap1Δ: The deletion of the pap1⁺ gene is not essential for high throughput screening, however it appears to make the cells more sensitive to both 5FOA and to drug treatment. This gene encodes a transcriptional activator that regulates the expression of ABC transporter genes. Loss of this gene may allow compounds to accumulate in S. pombe.
4. A mutation in a glucose/cAMP pathway gene: This was required for most, but not all strains in order to screen for PDE inhibitors. Mutations such as git3-14 and git11Δ cause a modest reduction in cAMP generation, which the git3A deletion causes a moderate reduction in cAMP generation, and the gpa2 disruption causes a significant reduction in cAMP generation. In order to carry out a PDE inhibitor screen, cells must be 5FOA-sensitive due to an insufficient cAMP level to repress fbp1 transcription. These various mutations were used to control cAMP levels.

Should a PDE be encountered that has such low activity that even loss of the gpa2 gene fails to confer 5FOA-sensitivity, there are two alternative strategies to develop a screening strain. One strategy includes introducing the PDE gene into S. pombe under the control of a stronger promoter than the cgs2 promoter. Such promoters can be the nmt1, nmt41 or the SV40 promoter. A second strategy includes introducing a deletion of the adenylate cyclase git2 gene into the strain so that there is no cAMP production. Such cells are 5FOA-sensitive regardless of the strength of the heterologously-expressed PDE gene (as shown FIG. 1, which indicates that a git2Δ cgs2-s1 mutant is 5FOA-senstive). In this case, one can determine a concentration of cAMP that is added to the medium to confer 5FOA-resistant growth to a strain lacking both adenylate cyclase and PDE activity, but is insufficient to confer growth to a strain that lacks adenylate cyclase, but expresses the weak target PDE. A PDE inhibitor is identified by its ability to re-establish 5FOA-resistant growth due to the addition of this low level of cAMP. To summarize, if a PDE is extremely weak, one can replace endogenous cAMP production with exogenous cAMP addition to give one complete control over the level of cAMP in the system.

Table 6 describes growth conditions prior to exposure to 5FOA medium that have been determined for various strains. Optimized growth conditions for additional strains can be determined using routine culture methods.

TABLE 6

Experimental Conditions Per PDE Strain

| | | Pregrowth (mM | Cell Density |
|---|---|---|---|
| Strain | PDE | cAMP + EMM) | (cell/ml) |
| CHP1113 | PDE4B3 | 0.5 | 5 × 10\hat{ }4 |
| CHP932 | Cgs2 | 2.5 | 1 × 10\hat{ }5 |
| LWP369 | PDE2A | 0.2 | 5 × 10\hat{ }4 |
| CHP1098 | PDE4A1 | 1 | 4 × 10\hat{ }5 |
| CHP1155 | PDE4A5 | 2.5 | 2 × 10\hat{ }5 |
| CHP1169 | PDE7A | 2.5 | 1 × 10\hat{ }5 |
| DDP16 | PDE8A | 0.5 | 2 × 10\hat{ }4 |
| CHP1167 | PDE4D3 | 0.5 mM | TBD |
| | | cGMP* | |
| CHP1179 | PDE1C4 | 1.1 mM | TBD |
| | | cGMP* | |

*Situations in which exogenous cAMP is not able to confer 5FOA-resistant growth have been observed, however cGMP can be used successfully.
TBD—To be determined.

### Method for PDE Inhibitor Screen

The following provides a general protocol for PDE inhibitor screening. Such a method, or similar methods are useful to screen the strains of the invention to identity PDE inhibitors.

Cells were pregrown in EMM medium [MP Biomedicals (Solon, OH), 3% glucose, filter-sterilized to avoid carmelization, which would introduce variability into the optical density of the medium] containing from 0 mM to 2.5 mM cAMP (or either 0.5 mM or 1.0 mM cGMP). This was to repress expression of the fbp1-ura4 reporter prior to exposure of cells to 5FOA medium. Cells were grown at 30° C. to exponential phase (approximately 10⁷ cells/ml). Cells were collected by centrifugation and resuspended in 5FOA medium, and 25 μl were transferred to 384-well microtiter dishes (untreated, with flat clear bottoms) that had been pre-filled with 25 μl 5FOA medium and pre-pinned with 100 nl of compounds (stock solutions were generally 10 mM). Starting cell concentrations ranged from 0.5×10⁵ to 4×10⁵ cells/ml depending on the screening strain. As appropriate, control plates received either 100 nl 10 mM rolipram (for rolipram-sensitive PDE4s) or DMSO. Other positive control dishes contained 5 mM cAMP in the 5FOA medium for PDEs that lack appropriate control compounds. Cultures were grown for 48 hours at 30° C., sealed in an airtight container with moist paper towels to prevent evaporation. Optical densities (OD₆₀₀) of cultures were measured using a microplate reader. Bioinformatic analysis of the results to determine composite Z scores was performed as previously described (1, 3).

### PDE Activator Screens

For a PDE activator screen, the starting strain must have a sufficiently high cAMP level so that repression of fbp1-ura4 transcription prevents growth in either EMM medium lacking uracil or SC medium lacking uracil. Generally, this means that the strain has an intact glucose/cAMP signaling pathway. If such a strain is still able to grow due to a high level of PDE activity, it is possible to reduce growth further by supplementing the medium with cAMP.

For the screen, cells are pregrown in EMM medium containing uracil (and possibly supplemented with cAMP). Exponential phase cells are collected by centrifugation and diluted to an appropriate concentration in EMM medium lacking uracil or SC medium lacking uracil. cAMP may be added to produce an appropriate reduction in growth. Cells are transferred into microtiter dishes containing the same growth medium as used to dilute the cells into which compounds have been pinned. Microtiter dishes are incubated at 30° C. in sealed containers to prevent evaporation. The time of incubation depends on growth of control strains, but will likely be between 24 and 72 hours. Incubation times are optimized for each strain. Optical densities (OD₆₀₀) of cultures will be measured using a microplate reader. Bioinformatic analysis of the results to determine composite Z scores will be performed as previously described (1, 3).

In addition to detecting PDE activators, this screen detects compounds that promote growth by inhibiting adenylate cyclase or protein kinase A (PKA), or by stimulating a stress-activated MAP kinase pathway involved in regulating fbp1 transcription. By comparing results from strains expressing different PDEs, such compounds can be eliminated from further study as they will promote growth in many, if not all, strains. In addition, measurement of cAMP levels before and after candidate drug addition distinguish among these various possibilities. Use of a control strain that lacks any PDE identifies compounds that reduce cAMP levels by a mechanism other than PDE stimulation.

### Screen for Biological Activators of PDEs

A screen for biological activators of a target PDE includes screening a cDNA library for genes that when expressed in S. pombe stimulate PDE activity to lower cAMP levels, thus stimulating growth in medium lacking uracil. As such, such an assay has many features similar to the chemical screen for PDE activators. The screening strain expresses a foreign PDE and possesses cAMP levels that are high enough to repress fbp1-ura4 transcription, so that stimulation of PDE activity lowers the cAMP level to de-repress fbp1-ura4. Desired strains for the assay have the lowest level of cAMP that is still sufficient to prevent single colony formation on medium lacking uracil. These strains are used as hosts to screen the cDNA library for biological activators of the target PDEs. These activators are identified by their ability to reduce cAMP levels, allowing single colony formation on SC-ura or EMM-ura medium.

This screen is carried out using a protocol previously used to identify plasmid insertions that disrupt chromosomal genes required for cAMP signaling and fbp1-ura4 repression (2). Host strains are transformed with the cDNA library and plated onto EMM-leucine to select for transformants. Rather than replica plating to SC-ura (this approach is not sufficiently sensitive as much less growth in required for regrowth on a replica plate than is required for single colony formation), colonies from individual transformation plates (targeting for approximately 10,000 colonies per plate) are collected in separate pools and replated at approximately 1,000,000 cells per plate onto SC-ura or EMM-ura. Colonies form on SC-ura or EMM-ura when a transformant from the EMM-leu plate carries a plasmid that increases fbp1-ura4 expression. If such a transformant is present on the initial transformation plate, then hundreds of colonies will form upon replating. This is easily distinguished from the few colonies that will form due to spontaneous mutations in genes required for cAMP signaling. The plasmids are introduced into S. pombe strains that do not express a target PDE as well as those that express other PDEs not used in the original screen. By determining the growth phenotypes of these transformants, plasmids that confer Ura⁺ growth by mechanisms other than the stimulation of the specific target PDE in question, are identified. Candidate plasmids that display specificity for a particular isoenzyme encode potential PDE activating proteins.

### REFERENCES FOR EXAMPLE 5

1. Franz, A. K., et al., J Am Chem Soc 129:1020-1.
2. Hoffman, C. S., and R. Welton. 2000. Biotechniques 28:532-6, 538, 540.
3. Kim, Y. K., et al., J Am Chem Soc 126:14740-5.

### Example 6

### Methods of Expressing a Camp PDE at a Higher Level than from the Yeast PDE Promoter

The method includes the introduction of a PDE into the plasmid pRH1 (Hoffman and Hoffman 2006), which carries two selectable markers. It has the S. cerevisiae LEU2 gene that complements S. pombe leu1 mutations and is transcribed from the SV40 promoter. It also has the S. pombe lys2 gene. The PDE gene is introduced into pRH1, replacing the LEU2 gene by gap repair transformation (Wang, Kao et al. 2004), so that the PDE gene is expressed from the SV40 promoter (this gives high level expression). Specifically, this is done by linearizing pRH1 within the LEU2 gene with an enzyme such as Bbs1 that cuts in LEU2, but not elsewhere in the plasmid. This linearized plasmid is co-transformed into a lys2⁻ mutant strain of S. pombe together with a PCR product that contains the PDE gene flanked by sequences from pRH1 that target the PDE gene to recombine with the plasmid upon uptake into the yeast cells. For example, to integrate clones obtained from the company OriGene, using priming sequences that are universal to the cloning vector, the following oligonucleotides are used:

Forward oligonucleotide

(SEQ ID NO: 29)

5′ ttccagaagtagtgaggaggcttttttggaggcctaggcttttgcaa
aaagctttgcaaaggcaccaaaatcaacgggac 3′
Reverse oligonucleotide

(SEQ ID NO: 30)

5′ tgaatgggcttccatagtttgaaagaaaaaccctagcagtactggca
agggagacattccttattaggacaaggctggtg 3′

S. pombe cells are plated onto EMM-lysine to select for Lys⁺ transformants. These colonies are pooled and the plasmids are rescued back to E. coli (Hoffman and Winston 1987), selecting for ampicillin-resistance. Individual transformants are checked by plasmid prep and restriction digestion to identify correct plasmids that carry the PDE gene in place of LEU2.

The cloned PDE is then stably introduced into the S. pombe genome by linearizing the plasmid within the lys2 gene on the plasmid and transforming a lys2-97 mutant strain (such as CHP1077) to Lys⁺. By linearizing the plasmid, integration by homologous recombination is greatly enhanced. One can find stable integrants by passaging the Lys⁺ transformants two or three times on nonselective medium (yeast extract agar; this can be done by simply replica plating) and then replica plating back to EMM-lysine medium. The stable Lys⁺ transformants (containing the plasmid integrated at the lys2 locus) will show solid growth on the EMM-Lys plate indicating that most of the cells retain the plasmid, while the original Lys⁺ transformants that did not have the plasmid integrated will show patchy growth, if any, on the EMM-Lys plate due to the high frequency of plasmid loss.

Once a strain carrying the integrated plasmid has been identified, screening strains are constructed by standard genetic crosses as described for the strains expressing PDE genes at the cgs2 locus.

The human PDE10A described in Example 5 herein, has also been put onto the plasmid to express it from the SV40 promoter using SEQ ID NOs:29 and 30. A resulting S. pombe transformant has been identified that has the plasmid integrated into the lys2 locus as described above.

### REFERENCES FOR EXAMPLE 6

1 Hoffman, C. S. and F. Winston (1987). Gene 57(2-3): 267-72.
2 Hoffman, R. L. and C. S. Hoffman (2006). Curr Genet. 49(6): 414-20.
3 Wang, L., R. Kao, et al. (2004). Methods 33: 199-205.

All patents, patent applications, and published references cited herein are hereby incorporated by reference in their entirety. It will be appreciated that various of the above-disclosed and other features and functions, or alternatives thereof, may be desirably combined into many other different systems or applications. Various presently unforeseen or unanticipated alternatives, modifications, variations, or improvements therein may be subsequently made by those skilled in the art which are also intended to be encompassed by the following claims.

TABLE 7
Group 22, 27

| Compound | | | | PDEs | | | | | |
|---|---|---|---|---|---|---|---|---|---|
| Alias | Structure | Smiles | Activity | 4A1 | 4A5 | 4B3 | 4D2 | 4D3 | 7A |
| 22 (4) | 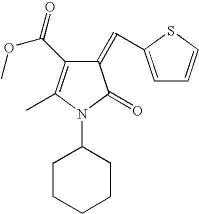 | COC(═O)C/1═C(C)N(C2CCCCC2)C(═O)\C1═C/c3cccs3 | 1 Assay (20 uM) 2 Assay (2 uM) ED50 cAMP Response (% rolipram) IC50 | 98.84 27.01 9.42 54.00 ND | −0.06 13.66 1.27 ND ND | 70.26 37.54 0.14 51.00 ND | ND ND ND 34.00 ND | ND 42.32 >200 12.00 ND | 132.82 57.22 >200 ND ND |
| 27 (3) | 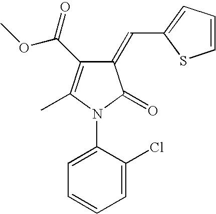 | COC(═O)C/1═C(C)N(C(═O)\C1═C/c2cccs2)c3ccccc3Cl | 1 Assay (20 uM) 2 Assay (2 uM) ED50 cAMP Response (% rolipram) IC50 | 64.18 17.41 200.00 78.00 100 nM | 65.67 12.63 13.00 ND ND | −0.26 22.05 3.80 71.00 ND | ND ND ND 11.00 ND | ND 0.00 >200 8.00 100 nm | 2.34 5.07 >200 ND ND |
| 27-1 (1) | 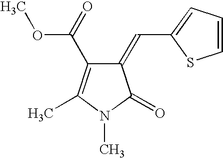 | COC(═O)C/1═C(C)N(C)C(═O)\C1═C/c2cccs2 | [symbol] uM) 2 Assay (2 uM) ED50 cAMP Response IC50 | ND ND 107.90 ND | ND ND 3.41 ND | ND ND >200 ND | ND ND >200 ND | ND ND >200 ND | ND ND >200 ND |
| 27-2 (2) | 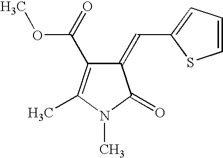 | COC(═O)C/1═C(C)N(CC(C)C)C(═O)\C1═C/c2cccc2 | 1 Assay (20 uM) 2 Assay (2 uM) ED50 cAMP Response IC50 | ND ND 4.83 ND | ND ND 0.75 ND | ND ND 2.66 ND | ND ND 30.57 ND | ND ND 68.40 ND | ND ND >200 ND |

(1) Strong PDEA5 inhibitor
(2) Strong PDE4A, 4B inhibitor; Moderate PDE4D inhibitor
(3) Strong PDE4A, 4B inhibitor
(4) Strong PDE4A, 4B, 7 inhibitor; Weak PDE4D inhibitor

| | | | | | | | | | |
|---|---|---|---|---|---|---|---|---|---|
| 27-3 (1) | 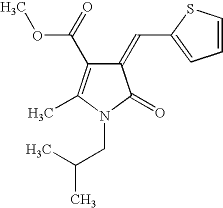 | COC(═O)C/1═C(C)N(CC(C)C)C(═O)\C1═C/c2cccs2 | 1 Assay (20 uM) 2 Assay (2 uM) ED50 cAMP Response IC50 | ND ND 7.63 ND | ND ND 0.85 ND | ND ND 3.84 ND | ND ND >200 ND | ND ND >200 ND | ND ND >200 ND |
| 27-4 (1) | 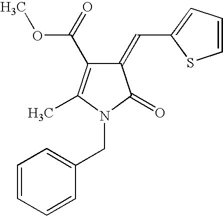 | COC(═O)C/1═C(C)N(C(═O)\C1═C/c2cccs2)c3cccc3 | 1 Assay (20 uM) 2 Assay (2 uM) ED50 cAMP Response IC50 | ND ND 27.66 ND | ND ND 1.33 ND | ND ND 3.57 ND | ND ND >200 ND | ND ND >200 ND | ND ND >200 ND |
| 27-5 (2) | 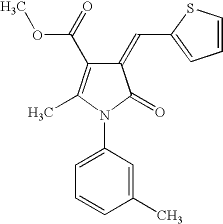 | C3═CSC(C═C2C(═O)N(CC1═CC═CC(C)═C1)C(C)═C2C(OC)═O)═C3 | 1 Assay (20 uM) 2 Assay (2 uM) ED50 cAMP Response IC50 | ND ND 1.01 ND | ND ND 0.23 ND | ND ND 2.04 ND | ND ND 17.15 ND | ND ND 14.74 ND | ND ND >200 ND |
| 27-6 (4) | 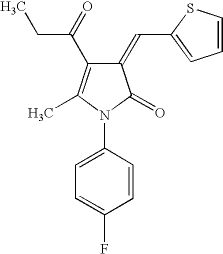 | C3═CSC(C═C2C(═O)N(C1═CC═C(F)C═C1)C(C)═C2C(OC)═O)═C3 | 1 Assay (20 uM) 2 Assay (2 uM) ED50 cAMP Response IC50 | ND ND 4.26 ND | ND ND 0.83 ND | ND ND 2.34 ND | ND ND >200 ND | ND ND >200 ND | ND ND 13.04 ND |
| 27-7 (3) | 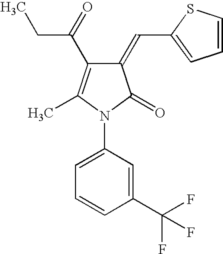 | C3═CSC(C═C2C(═O)N(C1═CC═CC(C(F)(F)F)═C1)C(C)═C2C(═O)OC)═C3 | 1 Assay (20 uM) 2 Assay (2 uM) ED50 cAMP Response IC50 | ND ND >200 ND | ND ND 2.31 ND | ND ND >200 ND | ND ND >200 ND | ND ND >200 ND | ND ND >200 ND |

(1) Strong PDE4A, 4B inhibitor
(2) Strong PDE4A, 4B inhibitor, Moderate PDE4D inhibitor
(3) Strong PDE4A5 inhibitor
(4) Strong PDE4A, 4B inhibitor, Moderate PDE7A inhibitor

| | | | | | | | | | |
|---|---|---|---|---|---|---|---|---|---|
| 27-8 (2) | 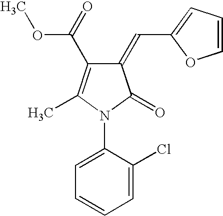 | COC(═O)C/1═C(C)N(C(═O)\C1═C/c2cccO2)c3cccc3Cl | 1 Assay (20 uM) 2 Assay (2 uM) ED50 cAMP Response IC50 | ND ND 2.74 ND | ND ND 1.55 ND | ND ND 18.38 ND | ND ND >200 ND | ND ND >200 ND | ND ND >200 ND |
| 2008, C21 (2) | 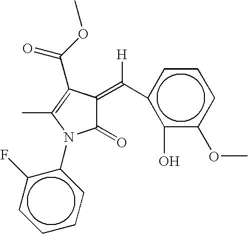 | COC(═O)C/1═C(C)N(C(═O)\C1═C/c2cccc(OC)c2O)c2ccccc3F | 1 Assay (20 uM) 2 Assay (2 uM) ED50 cAMP Response IC50 | 18.97 ND ND ND ND | 1.80 ND ND ND ND | 24.16 ND ND ND ND | ND ND ND ND ND | ND ND ND ND ND | −0.08 ND ND ND ND |
| 2008, K19 (1) | 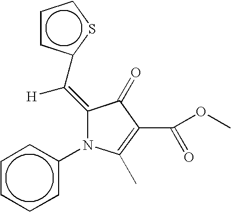 | COC(═O)C1═C(C)N(/C(═C/c2cccs2)/C1═O)c3ccccc3 | 1 Assay (20 uM) 2 Assay (2 uM) ED50 cAMP Response IC50 | −0.46 ND ND ND ND | −0.06 ND ND ND ND | −0.61 ND ND ND ND | ND ND ND ND ND | ND ND ND ND ND | 2.43 ND ND ND ND |
| 2008, O19 (1) | 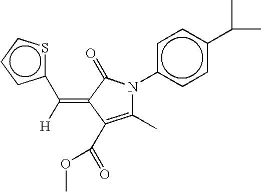 | COC(═O)C/1═C(C)N(C(═O)\C1═C/c2ccco2)c3ccc(cc3)C(C)C | 1 Assay (20 uM) 2 Assay (2 uM) ED50 cAMP Response IC50 | 0.80 ND ND ND ND | 1.73 ND ND ND ND | −2.99 ND ND ND ND | ND ND ND ND ND | ND ND ND ND ND | 2.78 ND ND ND ND |

(1) No activity under conditions used
(2) Strong PDE4A, 4B inhibitor [symbol]
indicates data missing or illegible when filed

TABLE 8
PDE 7A vs. 30-series
Group 30

| Compound | | | | PDEs | | | | | |
|---|---|---|---|---|---|---|---|---|---|
| Alias | Structure | Smiles | Activity | 4A1 | 4A5 | 4B3 | 4D2 | 4D3 | 7A |
| 30 KM03472 | 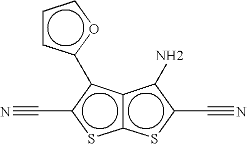 | Nc1c(C#N)sc2sc(C#N)c(c3ccco3)c12 (2) | [symbol] uM) 2 Assay (2 uM) ED50 cAMP Response IC50 | 11.40 4.56 ND 65.7 ND | 2.14 2.79 ND ND ND | 4.67 5.21 ND 67 ND | ND ND ND 7.5 ND | ND −0.55 ND 4 ND | 103.99 67.85 4 153.6 ND |
| 1441, G04 KM03473 30-2 | 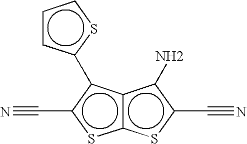 | (1) Nc1c(C#N)sc2sc(C#N)c(c3cccs3)c12 | [symbol] uM) 2 Assay (2 uM) ED50 cAMP Response IC50 | −0.2539 ND ND ND ND | ND ND ND ND ND | −0.3999 ND ND ND ND | ND ND ND ND ND | ND ND ND ND ND | ND ND ND ND ND |
| Maybridge: KM03484 30-4 | 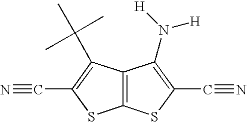 | CC(C)(C)c1c(C#N)sc2sc(C#N)c(N)c12 (3) | [symbol] uM) 2 Assay (2 uM) ED50 cAMP Response IC50 | ND ND ND ND ND | ND ND ND ND ND | ND ND ND ND ND | ND ND ND ND ND | ND ND ND ND ND | ND ND weak activity ND ND |
| 554, N12 KM03474 30-3 | 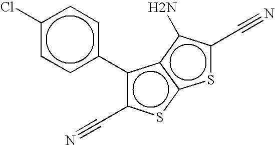 | Nc1c(C#N)sc2sc(C#N)c(c3ccc(Cl)cc3)c12 (3) | [symbol] uM) 2 Assay (2 uM) ED50 cAMP Response IC50 | ND ND ND ND ND | ND ND ND ND ND | 1.462 ND ND ND ND | ND ND ND ND ND | ND ND ND ND ND | ND ND inactive ND ND |
| 1441, C04 KM03459 30-1 | 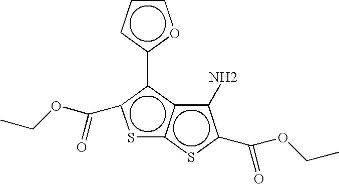 | CCOC(═O)c1sc2sc(C(═O)OCC)c(c3ccco3)c2c1N (4) | [symbol] uM) 2 Assay (2 uM) ED50 cAMP Response IC50 | 0.5758 ND ND ND ND | ND ND ND ND ND | 8.4406 ND ND ND ND | ND ND ND ND ND | ND ND ND ND ND | ND ND inactive ND ND |
| 2055, C21 | 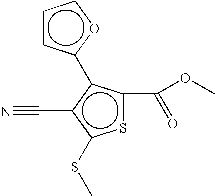 | COC(═O)c1sc(SC)c(C#N)c1c2ccco2 | [symbol] uM) 2 Assay (2 uM) ED50 cAMP Response IC50 | 15.8505 ND ND ND ND | 2.07 ND ND ND ND | 21.4955 ND ND ND ND | ND ND ND ND ND | ND ND ND ND ND | 0.2998 ND ND ND ND |
| 1449, A02 | 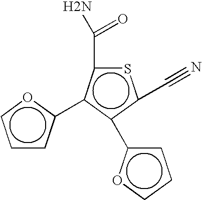 | NC(═O)c1sc(C#N)c(c2ccco2)c1c3ccco3 | [symbol] uM) 2 Assay (2 uM) ED50 cAMP Response IC50 | 1.3541 ND ND ND ND | ND ND ND ND ND | 1.8244 ND ND ND ND | ND ND ND ND ND | ND ND ND ND ND | ND ND ND ND ND |
| 2055, N01 | 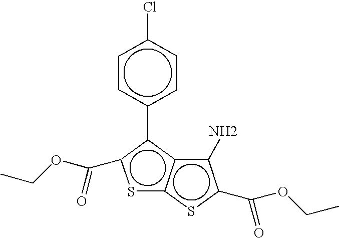 | CCOC(═O)c1sc2sc(C(═O)OCC)c(c3ccc(Cl)cc3)c2c1N | [symbol] uM) 2 Assay (2 uM) ED50 cAMP Response IC50 | 2.1835 ND ND ND ND | 0.582 ND ND ND ND | 1.8421 ND ND ND ND | ND ND ND ND ND | ND ND ND ND ND | 1.4276 ND ND ND ND |

(1) Weak PDE7A inhibitor
(2) Strong PDE7A inhibitor
(3) Little/no activity under conditions used
(4) Weak PDE4A inhibitor [symbol]
indicates data missing or illegible when filed

TABLE 9
Group 35

| Compound | | | | PDEs | | | | | |
|---|---|---|---|---|---|---|---|---|---|
| Alias | Structure | Smiles | Activity | 4A1 | 4A5 | 4B3 | 4D2 | 4D3 | 7A |
| 35 | 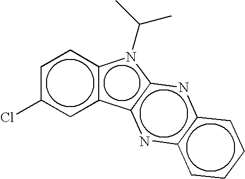 | CC(C)n1c2ccc(Cl)cc2c3nc4ccccc4nc13 | 1 Assay (20 uM) 2 Assay (2 uM) ED50 cAMP Response IC50 | 78.02 9.44 5.80 96.00 100 nM | 66.33 11.29 >200 ND ND | 0.17 11.39 >200 76.00 ND | ND ND >200 10 ND | ND −0.03 >200 3 35 nM | −0.61 −0.37 >200 ND ND |
| 41 | 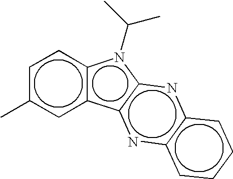 | CC(C)n1c2ccc(C)cc2c3nc4ccccc4nc13 | 1 Assay (20 uM) 2 Assay (2 uM) ED50 cANP Response IC50 | 106.90 ND 3.50 650 nM | 84.08 ND 0.40 ND | 200.51 ND 18.00 ND | ND ND >200 ND | ND ND >200 200 nM | 0.31 ND >200 ND |
| 1422, G02 | 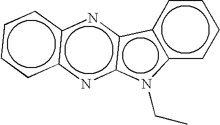 | CCn1c2ccccc2c3nc4ccccc4nc13 | 1 Assay (20 uM) 2 Assay (2 uM) ED50 cAMP Reponse IC50 | 70.89 ND ND ND ND | ND ND ND ND ND | 103.69 ND ND ND ND | ND ND ND ND ND | ND ND ND ND ND | ND ND ND ND ND |
| 1422, I02 | 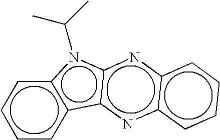 | CC(C)n1c2ccccc2c3nc4ccccc4nc13 | 1 Assay (20 uM) 2 Assay (2 uM) ED50 cAMP Response IC50 | 56.62 ND ND ND ND | ND ND ND ND ND | 99.04 ND ND ND ND | ND ND ND ND ND | ND ND ND ND ND | ND ND ND ND ND |
| 1422, K02 | 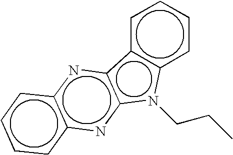 | CCCn1c2ccccc2c3nc4ccccc4nc13 | 1 Assay (20 uM) 2 Assay (2 uM) ED50 cAMP Response IC50 | 72.21 ND ND ND ND | ND ND ND ND ND | 115.27 ND ND ND ND | ND ND ND ND ND | ND ND ND ND ND | ND ND ND ND ND |

All compounds on this page are strong PDE4A, 4B inhibitors

| | | | | | | | | | |
|---|---|---|---|---|---|---|---|---|---|
| 1422, M02 (1) | 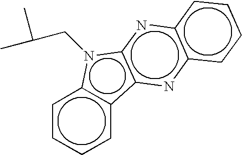 | CC(C)Cn1c2ccccc2c3nc4ccccc4nc13 | 1 Assay (20 uM) 2 Assay (2 uM) ED50 cAMP Response IC50 | 40.53 ND ND ND ND | ND ND ND ND ND | 86.04 ND ND ND ND | ND ND ND ND ND | ND ND ND ND ND | ND ND ND ND ND |
| 1427, P20 (3) | 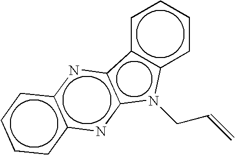 | C═CCn1c2ccccc2c3nc4ccccc4nc13 | 1 Assay (20 uM) 2 Assay (2 uM) ED50 cAMP Response IC50 | 31.38 ND ND ND ND | ND ND ND ND ND | 103.45 ND ND ND ND | ND ND ND ND ND | ND ND ND ND ND | ND ND ND ND ND |
| 1429, L06 (4) | 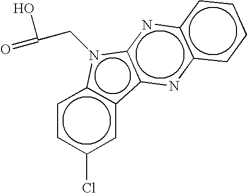 | OC(═O)Cn1c2ccc(Cl)cc2c3nc4ccccc4nc13 | 1 Assay (20 uM) 2 Assay (2 uM) ED50 cAMP Response IC50 | 29.15 ND ND ND ND | ND ND ND ND ND | 41.77 ND ND ND ND | ND ND ND ND ND | ND ND ND ND ND | ND ND ND ND ND |
| 2013, I20 (2) | 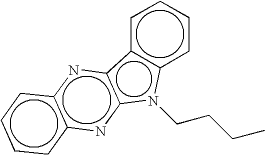 | CCCCn1c2ccccc2c3nc4ccccc4nc13 | 1 Assay (20 uM) 2 Assay (2 uM) ED50 cAMP Response IC50 | 80.55 ND ND ND ND | 71.82 ND ND ND ND | 0.29 ND ND ND ND | ND ND ND ND ND | ND ND ND ND ND | −0.84 ND ND ND ND |
| 2015, L04 (5) | 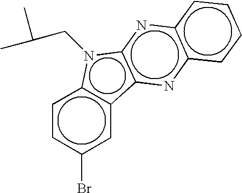 | CC(C)Cn1c2ccc(Br)cc2c3nc4ccccc4nc13 | 1 Assay (20 uM) 2 Assay (2 uM) ED50 cAMP Response IC50 | 13.51 ND ND ND ND | 57.69 ND ND ND ND | 1.17 ND ND ND ND | ND ND ND ND ND | ND ND ND ND ND | −0.79 ND ND ND ND |

(1) Strong PDE4A inhibitor
(2) Strong PDE4A inhibitor
(3) Strong 4B, Moderate 4A inhibitor
(4) Moderate PDE4A, 4B inhibitor
(5) Moderate 4B inhibitor

| | | | | | | | | | |
|---|---|---|---|---|---|---|---|---|---|
| 2015, P06 (2) | 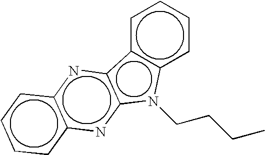 | CCc1ccc2n(CC)c3nc4ccccc4nc3c2c1 | 1 Assay (20 uM) 2 Assay (2 uM) ED50 cAMP Response IC50 | 23.52 ND ND ND ND | 51.82 ND ND ND ND | 0.13 ND ND ND ND | ND ND ND ND ND | ND ND ND ND ND | 1.07 ND ND ND ND |
| 2017, C18 (3) | 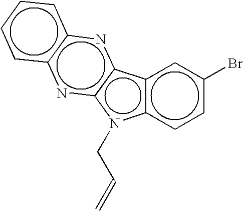 | Brc1ccc2n(CC═C)c3nc4ccccc4nc3c2c1 | 1 Assay (20 uM) 2 Assay (2 uM) ED50 cAMP Response IC50 | 49.17 ND ND ND ND | 69.70 ND ND ND ND | 56.91 ND ND ND ND | ND ND ND ND ND | ND ND ND ND ND | −1.58 ND ND ND ND |
| 2019, G03 (4) | 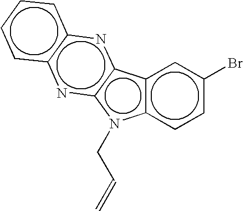 | C(Cn1c2ccccc2c3nc4ccccc4nc13)c5ccccc5 | 1 Assay (20 uM) 2 Assay (2 uM) ED50 cAMP Response IC50 | 36.37 ND ND ND ND | 49.61 ND ND ND ND | 83.22 ND ND ND ND | ND ND ND ND ND | ND ND ND ND ND | 4.47 ND ND ND ND |
| 2033, H06 (1) | 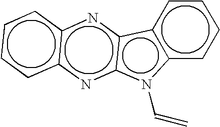 | C═Cn1c2ccccc2c3nc4ccccc4nc13 | 1 Assay (20 uM) 2 Assay (2 uM) ED50 cAMP Response IC50 | 29.62 ND ND ND ND | 66.58 ND ND ND ND | 103.11 ND ND ND ND | ND ND ND ND ND | ND ND ND ND ND | 0.63 ND ND ND ND |

(1) Moderate to strong PDE4A, 4B inhibitor
(2)-(4) Moderate PDE4A, 4B inhibitors

TABLE 10
Group 39

| Compound | | | | PDEs | | | | | |
|---|---|---|---|---|---|---|---|---|---|
| Alias | Structure | Smiles | Activity | 4A1 | 4A5 | 4B3 | 4D2 | 4D3 | 7A |
| 39 | 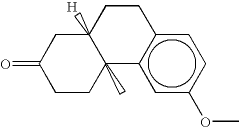 | COc1ccc2CC[C@@H]3CC(═O)CC[C@]3(C)c2c1 | 1 Assay (20 uM) 2 Assay (2 uM) ED50 cAMP Response IC50 | −0.53 ND >200 68.10 ND | 24.92 ND >200 38.30 ND | 25.84 ND 51.75 37.50 ND | ND ND >200 8.50 ND | −0.74 ND >200 4.10 ND | 135.37 ND 5.77 55.30 ND |
| 2158J12 (2) | 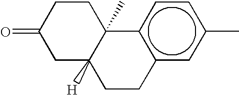 | Cc1ccc2c(CC[C@H]3CC(═O)CC[C@@]32C)c1 | 1 Assay (20 uM) 2 Assay (2 uM) ED50 cAMP Response IC50 | 81.97 2.18 ND ND ND | 113.27 7.00 ND ND ND | 37.13 8.18 ND ND ND | ND ND ND ND ND | 14.84 40.17 ND ND ND | 74.45 29.01 ND ND ND |
| 11 (3) | 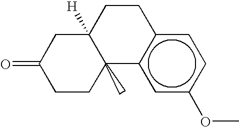 | COc1ccc2CC[C@H]3CC(═O)CC[C@]3(C)c2c1 | 1 Assay (20 uM) 2 Assay (2 uM) ED50 cAMP Response (%BRL50481) IC50 | −0.01 ND ND ND ND | −0.32 ND ND ND ND | 2.22 ND ND ND ND | ND ND ND ND ND | −0.58 ND ND ND ND | 88.24 ND 22.00 89.00 ND |
| 2067M03 (1) | 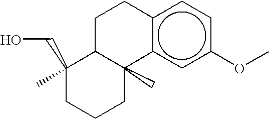 | COc1ccc2CCC3[C@@](C)(CO)CCC[C@]3(C)c2c1 | 1 Assay (20 uM) 2 Assay (2 uM) ED50 cAMP Response IC50 | 11.56 5.63 ND ND ND | 16.94 8.43 ND ND ND | 13.34 19.00 ND ND ND | ND ND ND ND ND | ND 1.24 ND ND ND | 104.31 8.65 ND ND ND |

(1) Strong PDE7A inhibitor, Weak to moderate PDE4A, 4B inhibitor
(2) Strong PDE7A and PDE4s (including D) inhibitor
(3) Strong PDE7A inhibitor
(4) Strong PDE7A inhibitor, Weak to moderate PDE4A, 4B inhibitor

TABLE 11
Group 26

| Compound | | | | PDEs | | | | | | Human |
|---|---|---|---|---|---|---|---|---|---|---|
| Alias | Structure | Smiles | Activity | 4A1 | 4A5 | 4B3 | 4D2 | 4D3 | 4D3 | 4A1 |
| 26 (1) | 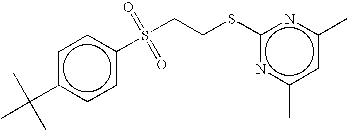 | Cc1cc(C)nc(SCCS(═O)(═O)c2ccc(cc2)C(C)(C)C)n1 | 1 Assay (20 uM) 2 Assay (2 uM) ED50 cAMP Response (% rolipram) IC50 | 99.87 26.63 2.44 96.00 500 nM | 70.97 13.13 0.24 57.30 ND | 0.39 12.54 0.13 92.00 ND | ND ND ND 17.00 ND | ND 0.97 >200 14.00 500 nm | 0.58 0.13 >200 27.90 ND | ND ND 4.00 ND ND |
| 1428-P07 26-2 (2) | 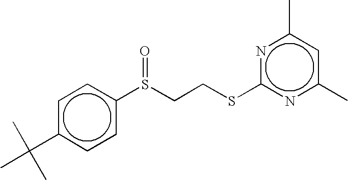 | Cc1cc(C)nc(SCCS(═O)c2ccc(cc2)C(C)(C)C)n1 | 1 Assay (2 uM) 2 Assay (2 uM) ED50 cAMP Response (% rolipram) IC50 | 53.44 ND ND ND | ND ND ND ND ND | 103.15 ND ND ND ND | ND ND ND ND ND | ND ND ND ND ND | ND ND ND ND ND | ND ND 24.00 ND ND |
| 26-3 | 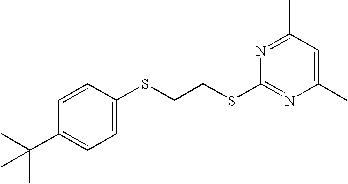 | Cc1nc(SCCSc2ccc(cc2)C(C)(C)C)nc(C)c1 | 1 Assay (20 uM) 2 Assay (2 uM) ED50 cAMP Response (% rolipram) IC50 | ND ND ND ND | ND ND ND ND ND | ND ND ND ND ND | ND ND ND ND ND | ND ND ND ND ND | ND ND ND ND ND | ND ND >200 ND ND |
| 1437-E15 | 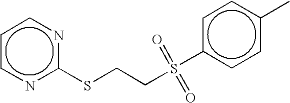 | Cc1ccc(cc1)S(═O)(═O)CCSc2ncccn2 | 1 Assay (20 uM) 2 Assay (2 uM) ED50 cAMP Response (% rolipram) IC50 | −0.15 ND ND ND ND | ND ND ND ND ND | 3.19 ND ND ND ND | ND ND ND ND ND | ND ND ND ND ND | ND ND ND ND ND | ND ND ND ND ND |
| 1437-C15 | 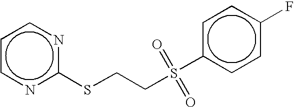 | Fc1ccc(cc1)S(═O)(═O)CCSc2ncccn2 | 1 Assay (20 uM) 2 Assay (2 uM) ED50 cAMP Response (% rolipram) IC50 | 0.65 ND ND ND ND | ND ND ND ND ND | 4.18 ND ND ND ND | ND ND ND ND ND | ND ND ND ND ND | ND ND ND ND ND | ND ND ND ND ND |

(1) Strong PDE4A, 4B inhibitor
(2) Moderate 4A inhibitor

What is claimed is:

1. A composition comprising (a) a phosphodiesterase (PDE) inhibitor represented by any one of the formulas of groups I to VI, wherein the PDE inhibitor inhibits PDE4 activity, PDE7 activity or both PDE4 activity and PDE7 activity and (b) a pharmaceutically acceptable carrier.

2. The composition of claim 1, wherein the composition specifically inhibits PDE4A activity PDE4B and/or PDE7A activity.

3. A method for treating a PDE-associated disease or condition in an individual, comprising:
administering to an individual in need thereof a therapeutically effective amount of a composition of claim 1 or 2 to treat the PDE-associated disease or condition in the subject.

4. The method of claim 3, wherein the individual is human.

5. The method of claim 3, wherein the PDE-inhibiting compound is linked to a targeting molecule.

6. The method of claim 3, wherein the PDE-inhibiting compound is administered prophylactically to an individual at risk of having a PDE-associated disease or disorder.

7. The method of claim 3, wherein the PDE-inhibiting compound is administered in combination with an additional drug for treating a PDE-associated disease or disorder.

8. The method of claim 3, wherein the PDE-associated disease or disorder is a neurodegenerative disorders, penile erectile dysfunction, anxiety, depression, Alzheimer's disease, Parkinson's disease, Huntington's disease, schizophrenia, psychosis, sepsis, asthma, chronic obstructive pulmonary disease, pulmonary hypertension, renal disease, stroke, rhinitis, psoriasis, memory loss, chronic lymphocytic leukemia, prostate cancer, thyroid disease, male hypogonadism, cardiac disease, diabetes, obesity, multiple sclerosis, rheumatoid arthritis, osteoporosis, or cystic fibrosis.

9. A method for increasing the level of a substrate of a PDE in a cell or tissue, comprising:
contacting the cell or tissue with an effective amount of a PDE-inhibiting compound represented by any one of the formulas of groups I to V or an analog, derivative, or variant thereof that inhibits PDE activity to increase, whereby PDE4 activity or PDE7 activity is inhibited and the level of the substrate of the PDE in the cell or tissue is increased.

10. The method of claim 9, wherein the substrate is cAMP or cGMP.
